(19) United States
(12) Patent Application Publication
(10) Pub. No.: US 2010/0317002 A1
(43) Pub. Date: Dec. 16, 2010

(54) METHODS AND KITS FOR DIAGNOSING LUNG CANCER

(75) Inventors: Michal Daniely, Ganei Tikva (IL); Lea Madi, Rishon-LeZion (IL); Tal Kaplan, Gan-Yavne (IL); Boaz Pal, Tel-Aviv (IL); Yuval Harari, D.N. Emek Soreq (IL); Tanweer M. Zaidi, Houston, TX (US); Ricardo L. Fernandez, Houston, TX (US); Jinping Zhang, Houston, TX (US); Ruth Katz, Houston, TX (US)

Correspondence Address: MARTIN D. MOYNIHAN d/b/a PRTSI, INC., P.O. BOX 16446, ARLINGTON, VA, 22215, US

(73) Assignee: Bioview Ltd., Nes Ziona (IL)

(21) Appl. No.: 12/682,594

(22) PCT Filed: Oct. 6, 2008
(86) PCT No.: PCT/IL2008/001322

§ 371 Date: Aug. 31, 2010

Related U.S. Application Data
(60) Provisional application No. 60/960,735, filed on Oct. 11, 2007.

(51) Int. Cl. C12Q 1/68

(52) U.S. Cl. 435/6

(57) ABSTRACT

Provided is a method of identifying a genetically abnormal cell in a sputum sample, the method comprising: (a) staining a sputum sample using a morphological stain so as to identify a lower airway tract cell or lung cell in the sputum sample; and (b) staining the sputum sample using fluorescent in situ hybridization (FISH) so as to identify in the lower airway tract cell or lung cell a genetic abnormality in at least one of human chromosome 3p22.1 and 10q22-23, thereby identifying the genetically abnormal cell in the sputum sample. Also provided are methods and kits of diagnosing lung cancer by detecting a presence of genetically abnormal cells above a predetermined threshold in a sputum sample.

### FIELD AND BACKGROUND OF THE INVENTION

The present invention, in some embodiments thereof, relates to methods and kits of identifying genetically abnormal cells in a sputum sample and diagnosing lung cancer.

Lung cancer is the leading cause of cancer deaths worldwide with a still increasing incidence. Despite decades of research, prognosis is still poor and lung cancer patient have 85% chance of death within 5 years. Symptoms in the early stages of lung cancer are rarely seen and the majority of patients have locally advanced stage III or IV disease at diagnosis. For many patients, successful treatment remains elusive, because advanced tumors are often not operable, and may also be resistant to tolerable doses of radiotherapy and chemotherapy. In contrast, individuals with early stage disease can achieve cure through surgical resection. Because of this dichotomy in outcome associated with stage at diagnosis, there has been persistent interest in designing and testing methods for early detection of lung cancer.

The initiation and progression of lung cancer is associated with genetic abnormalities including point mutations, allelic loss and methylation of tumor suppressor genes. However, to date, none of these abnormalities has proven a promising biomarker for early detection of lung cancer, prediction of prognosis or determination of eligibility for clinical intervention. In addition, during the development of lung cancer, several cytological changes occur, which are associated with the transition from mild, moderate, and marked atypia, to carcinoma in situ and then to invasive carcinoma. These changes represent cellular aspects of toxic damage of respiratory tract epithelium which may result from smoking (nicotine) or exposure to radon gas.

Attempts to early diagnose lung cancer include sputum cytology, conventional chest x-ray, and helical computed tomography (CT) scanning. However, to date, the results of such screening tests have been controversial because of either low sensitivity and accuracy or the uncertain significance of their findings (Melamed M R., 2000). In addition, samples obtained by fine needle aspiration (FNA), tissue biopsy, bronchoscopically procured brush, wash, or lavage were subject to cytological or FISH analyses [Jiang F., et al., 2005; Fernandez R L., et al., 2006; Barkan G A., et al., 2005; U.S. Pat. Appl. No. 20060078885 to Katz R., et al.). Chromosomal aberrations in human chromosomes 3p22.1 and 10q22-23 were found to be associated with lung cancer (WO 00212563A2 to Katz R., et al.).

The so-called molecular field cancerization process likely results from multiple clonal abnormalities arising within respiratory epithelial cells exposed to carcinogenic substances from tobacco smoke and other pollutants and reflects genetic predisposition to reduced DNA repair capacity. The presence of concurrent cytologic atypia in sputum cells, especially moderate and severe dysplasia, is also believed to reflect this field effect and was shown to be substantially associated with an increased risk of developing lung cancer (Prindiville S A., et al., 2003).

The present inventors have previously developed the Duet™ system for scanning in Bright field and fluorescent modes (WO0049391A1). This system was successfully applied for multiple myeloma follow-up, hematological diseases, and bladder cancer recurrence (Hardan et al., 2004; Shimoni et al., 2002; Daniely et al., 2005; US 2004-0197839 to Daniely M. et al.).

Additional background art includes WO0212563A2 (to KATZ, R et al.), WO0626714 (to KATZ, R et al.), WO07087612A2 (to KATZ, R et al.), Nymark P., et al., 2006; Girard L., et al., 2000.

### SUMMARY OF THE INVENTION

According to an aspect of some embodiments of the present invention there is provided a method of identifying a genetically abnormal cell in a sputum sample, the method comprising: (a) staining a sputum sample using a morphological stain so as to identify a lower airway tract cell or lung cell in the sputum sample; and (b) staining the sputum sample using fluorescent in situ hybridization (FISH) so as to identify in the lower airway tract cell or lung cell a genetic abnormality in at least one of human chromosome 3p22.1 and 10q22-23, thereby identifying the genetically abnormal cell in the sputum sample.

According to an aspect of some embodiments of the present invention there is provided a method of diagnosing lung cancer in a subject comprising: (a) staining a sputum sample of the subject with a morphological stain so as to identify lower airway tract cells or lung cells in the sputum sample; (b) staining the sputum sample with FISH so as to identify a genetic abnormality in at least one of human chromosome 3p22.1 and 10q22-23 in the lower airway tract cells or lung cells identified in step (a), wherein a percentage or number above a predetermined threshold of the lower airway tract cells or lung cells having the genetic abnormality is indicative of the lung cancer, thereby diagnosing the lung cancer in the subject.

According to an aspect of some embodiments of the present invention there is provided a method of diagnosing lung cancer in a subject, comprising: (a) staining a sputum sample with a morphological stain so as to identify lower airway tract cells or lung cells in the sputum sample; (b) staining the sputum sample with FISH so as to identify a genetic abnormality in at least one of human chromosome 3p22.1 and 10q22-23 in cells of the sputum sample, wherein a percentage or number above a predetermined threshold of: (i) the lower airway tract cells or lung cells of the sputum sample identified in step (a) having the genetic abnormality; or (ii) the cells of the sputum sample having the genetic abnormality; is indicative of the lung cancer, thereby diagnosing the lung cancer in the subject.

According to an aspect of some embodiments of the present invention there is provided a kit for diagnosing lung cancer, the kit comprising a morphological stain and a FISH probe specific for human chromosome 3p22.1 and/or 10q22-23.

According to some embodiments of the invention, the cells of the sputum sample comprise lower airway tract cells, lung cells, squamous epithelial cells and/or blood cells.

According to some embodiments of the invention, the method further comprising: (c) imaging the lower airway tract cell or lung cell with at least two imaging modalities, thereby identifying the genetic abnormality in the cell.

According to some embodiments of the invention, the method further comprising: (c) imaging the lower airway tract cells or lung cells with at least two imaging modalities, thereby identifying genetic abnormalities in the lower airway tract cells or lung cells.

According to some embodiments of the invention, the imaging is effected simultaneously.

According to some embodiments of the invention, the imaging is effected using an automated image analysis device capable of at least dual imaging.

According to some embodiments of the invention, the kit further comprises instructions for use in diagnosing lung cancer.

According to some embodiments of the invention, the kit for diagnosing lung cancer in a sputum sample.

According to some embodiments of the invention, the sputum sample is induced by saline inhalation.

According to some embodiments of the invention, the instructions comprise a predetermined threshold of a percentage or number of lower airway tract cell or lung cell having a genetically abnormality in the human chromosome 3p22.1 and/or 10q22-23 which is indicative of positive diagnosis of lung cancer.

According to some embodiments of the invention, the sputum sample is obtained from a subject at risk of developing lung cancer.

According to some embodiments of the invention, the subject is at risk of developing lung cancer.

According to some embodiments of the invention, the lung cancer comprises non-small cell lung cancer.

According to some embodiments of the invention, the lung cancer comprises metastatic lung cancer.

According to some embodiments of the invention, the morphological stain is selected from the group consisting of May-Grünwald-Giemsa, Giemsa, Papanicolaou, Diff-Quick, and Hematoxylin-Eosin.

According to some embodiments of the invention, the FISH is effected using a FISH probe specific to human chromosome 3p22.1 and a FISH probe specific to human chromosome 10q22-23.

According to some embodiments of the invention, the FISH is effected using at least three FISH probes.

According to some embodiments of the invention, the FISH is effected using at least four FISH probes.

According to some embodiments of the invention, the FISH is effected using a FISH probe specific to human chromosome 3p22.1, a FISH probe specific to human chromosome 10q22-23 and a FISH probe specific to human chromosome 10.

According to some embodiments of the invention, the FISH is effected using a FISH probe specific to human chromosome 3p22.1, a FISH probe specific to human chromosome 10q22-23 and a FISH probe specific to human chromosome 3.

According to some embodiments of the invention, the FISH is effected using a FISH probe selected from a group of probes specific to human chromosome 3p22.1, human chromosome 10q22-23, human chromosome 3 and human chromosome 10.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F are microscope images depicting cells of a sputum sample analyzed by FISH (FIGS. 1A, B, C and D) or Papanicolaou's stain (FIGS. 1E and F). A non-induced sputum sample was obtained from a 52-year-old healthy control individual, who never smoked and was subject to morphological staining (using Papanicolaou's stain) and FISH staining (using probes specific to the 3p22.1, 10q22-23, centromeric region of chromosome 3 and 10). FIGS. 1A-B—FISH analysis of squamous epithelial cells depicting diploid (two) signals for 3p22.1 (green) and centromeric 3 (orange); FIGS. 1C-D—FISH analysis of squamous epithelial cells depicting diploid signals for 10q22-23 (SFTPA1 gene; green) and centromeric 10 (red); FIGS. 1E-F—Morphological staining of squamous epithelial cells in sputum depicting normal morphology with unremarkable nuclear features and thin homogenous cytoplasm (Papanicolaou's stain; original magnification 400×). Total FISH abnormalities for 3p22.1, centromeric 3, 10q22-23, and centromeric 10 probes in all analyzed cells of the sputum sample (at least 100 epithelial cells) were 6.7%, which results along with cytology parameters analyzed on parallel slides in a cancer risk probability score of 0.064.

FIGS. 2A-D are microscope images depicting cells of a sputum specimen analyzed by FISH (FIGS. 2A and B) or Papanicolaou's stain (FIGS. 2C and D). A non-induced sputum sample was obtained from a high-risk 61-year-old male individual having a smoking history of 100 pack-year and a CT scan negative for lung cancer. The cells of the sputum sample were stained with Papanicolaou's stain or FISH using probes specific to 3p22.1, 10q22-23, centromere 3 and centromere 10. FIG. 2A—FISH analysis demonstrates a deletion of the 3p22.1 chromosomal locus (only one green signal; see green arrow pointing at the signal) relative to two copies of the centromeric region of chromosome 3 (two red signals; see red arrows pointing at the signals). FIG. 2B—FISH analysis demonstrates loss of the 10q22-23 locus (only one green signal; see green arrow pointing at the signal) relative to two copies of the centromeric region of chromosome 10 (see two red signals). Magnification of images in FIGS. 2A and B are ×630 (oil immersion objective). The total FISH abnormalities for 3p22.1, centromeric regions 3 and 10, and 10q22-23 were 15.5%. FIGS. 2C-D—Cytologic examination using Papanicolaou's stain shows cells of moderate to severe dysplasia with hyperchromatic irregular nuclei and keratinized thickened orange cytoplasm (FIG. 2C) and atypical squamous metaplasia (FIG. 2D). Magnification of images in FIGS. 2C-D are ×400. The calculated cancer risk probability score is 0.844.

FIGS. 3A-E are microscope images depicting cells of a sputum specimen analyzed by FISH (FIGS. 3A and B) or Papanicolaou's stain (FIGS. 3C, D and E). A non-induced sputum sample was obtained from a 67-year-old female, non-smoker, with a family history of lung cancer, who had stage III adenocarcinoma (peripheral lesion). The cells of the sputum sample were stained with Papanicolaou's stain or FISH using probes specific to 3p22.1, 10q22-23, centromere 3 and centromere 10. FIGS. 3A-B—Actual view (FIG. 3A) and “synthetic” view (i.e., an image created by the image analysis system describing the algorithm analysis of the image) (FIG. 3B) of FISH analysis demonstrating deletions of 3p22.1 (as indicated by the loss of one green signal) relative to two copies of the centromeric region of chromosome 3 (2 red signals). Based on the FISH analysis the total deletion 10q was 5.69%, centromeric 10 monosomy was 2.43%, and deletion 3p22.1 was 6.79%. Total number of FISH abnormalities involving chromosomes 3 and 10 were 15.89%. FIGS. 3C, D and E—Cytologic examination (Papanicolaou's stain) demonstrating extensive moderately and severely dysplastic cells (arrows). Magnification of the images shown in FIGS. 3C-E are ×400. The calculated cancer risk probability score is =0.817. This patient died with metastatic disease 24 months later.

FIGS. 4A-D are microscope images depicting cells of a sputum specimen analyzed by FISH (FIGS. 4C and D) or Papanicolaou stain (FIGS. 4A and B). A non-induced sputum sample was obtained from a 63-year-old man with a history of 50 pack-years of smoking. The cells of the sputum sample were stained with Papanicolaou or FISH using probes specific to 3p22.1, 10q22-23, centromere 3 and centromere 10. FIGS. 4A-B—Morphological staining demonstrating cells of moderate and severe dysplasia (arrows) (Papanicolaou's stain; original magnification ×400). FIG. 4C—FISH analysis demonstrating deletion of 3p22.1 (one green signal) relative to two copies of the centromeric probe of chromosome 3 (two red signals); FIG. 3D-FISH analysis demonstrating deletions of 10q22-23 (one green signal) relative to two copies of the centromeric probe of chromosome 10 (two red signals). The total 3p22.1 deletions were 5.63% and the total 10q22-23 deletions were 3.78%. Total FISH abnormalities of chromosomes 3 and 10 were 9.4%. The calculated cancer risk probability score is=0.830.

FIG. 5 is a graph depicting receiver operator curve (ROC) showing sensitivity and specificity of sputum test when cytology and FISH variables are combined. The ROC curve is based on FISH variables and cytology diagnosis. Y axis=sensitivity; X axis=specificity. The area under curve=0.822.

FIG. 6 is a graph depicting receiver operator curve (ROC) based on cytology, FISH or combining FISH and cytology variables (determined independently on parallel slides). Y axis=sensitivity; X axis=specificity. Shown are the sensitivity and specificity of sputum test when cytology and FISH variable are combined (red curve), FISH variables only (green curve) and cytology variable only (blue curve). The area under the curve equals to 0.822 when combining FISH variables with cytology (red), 0.682 based on the FISH variable alone (green) and 0.742 based on the cytological analysis alone.

FIGS. 7A-J depict various cells stained with Giemsa which may be present in a sputum sample. FIGS. 7A-I—depict cells which are relevant for the target scan since they are derived from the lower airway tract and the lungs, and FIG. 7J depicts a cell which is not-relevant for the target scan since it is derived from the upper airway tract. FIGS. 7A, 7B and 7C—metaplastic cells; FIGS. 7D, 7E and 7F—atypical cells; FIGS. 7G and 7H—lung macrophages; FIG. 7I—A respiratory epithelial cell; FIG. 7J—A squamous epithelial cell (derived from the upper airway tract).

FIGS. 8A-F are images of cells which were subject to the combined analysis (target scan) using a morphological Giemsa staining (FIGS. 8A, 8C and 8E) followed by FISH analysis (FIGS. 8B, 8D and 8F) with the 3p22.1 (green), 10q22-23 (red) and CEP10 (centromere of chromosome 10; aqua) probes. FIGS. 8A-B—bright field (FIG. 8A) and dark field (FIG. 8B) images of the same single cell (a respiratory epithelial cell) exhibiting two green signals (one is underneath the aqua signal), two aqua signals and one red signal, demonstrating a deletion of 10q22-23. FIGS. 8C-D—bright field (FIG. 8C) and dark field (FIG. 8D) images of the same single cell (a respiratory epithelial cell) exhibiting four green signals, three aqua signals and three red signals, demonstrating polysomy. FIGS. 8E-F—bright field (FIG. 8E) and dark field (FIG. 8F) images of the same single cell (a metaplastic cell) exhibiting three green signals, two aqua signals and two red signals, demonstrating amplification of 3p22.1. Magnifications used in FIGS. 8A, 8C and 8E—×20 objective; Magnifications used in FIGS. 8B, 8D and 8F—×60 objective.

FIG. 9 is chart plot describing results from 71 sputum samples analyzed by 3-color target FISH. The x axis represents the number of targets (cells-of-interest) analyzed per sputum sample; the y axis represents the “scoring index”. This index reflects the percentage of FISH aberrant cells out of the “target cells” identified in the sample and the cutoff for a positive result depends on the total number of “target cells” that were scored. The cutoff for a positive diagnosis is 7.5. It can be seen that all cancer patients, except one, are located above the threshold while the majority of healthy population (either smokers and non-smokers) are located below the threshold.

FIGS. 10A-C depict respiratory cells stained with Papanicolaou. FIG. 10A—ciliated cell; FIG. 10B—nonciliated bronchiolar (Clara) cell; FIG. 10C—goblet cell.

FIGS. 10D-E depict cells found in the epithelium of the alveoli stained with Papanicolaou. FIG. 10D—alveolar macrophage; FIG. 10E—alveolar type II cell.

FIGS. 11A-F depict cells stained with Papanicolaou which may be present in sputum sample. FIG. 11A—metaplastic cell; FIG. 11B—atypical cancer cell; FIG. 11C—atypical respiratory cell; FIG. 11D—intermediate squamous cell (a normal squamous cell which is not located on the surface of the tissue (superficial) but below the surface); FIG. 11E—squamous normal cell; FIG. 11F—superficial squamous cell.

FIG. 12 is a schematic illustration depicting the respiratory system including the lungs with alveolie, bronchioles, bronchus, trachea, larynx and pharynx.

FIGS. 13A-B depict Type I and II alvelolie cells.

FIG. 14 depicts cells of a blood smear showing (a) erythrocytes (red blood cells), (b) neutrophil; (c) eosinophil; and (d) lymphocyte.

FIG. 15 depicts atypically squamous cells characterized by deeply keratin, hyperchromatic nuclei, suspicious of squamous cell carcinoma.

### DESCRIPTION OF SPECIFIC EMBODIMENTS OF THE INVENTION

The present invention, in some embodiments thereof, relates to methods and kits of identifying genetically abnormal cells in a sputum sample and diagnosing lung cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

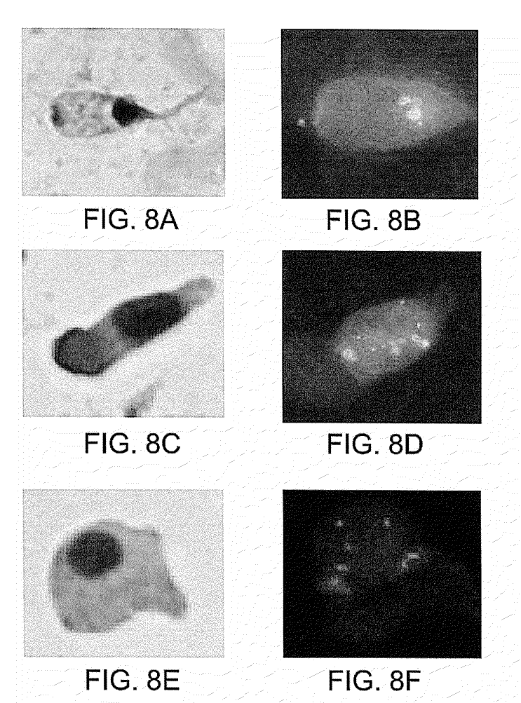

While reducing the present invention to practice, the present inventors have uncovered a novel, non-invasive method of identifying genetically abnormal cells in sputum samples, which can be efficiently employed in high sensitive detection of lung cancer. Thus, as shown in FIGS. 8A-B, 8C-D, 8E-F and described in Examples 3-4 of the Examples section which follows, the present inventors have stained cells of a sputum sample by two stains: a morphological stain which enables visualization of the morphological characteristics of a cell and classification thereof (e.g., type, level of differentiation, presence of abnormal morphological changes such as those found in atypic, metaplastic or dysplastic cells), and a subsequent FISH stain which enables determination of chromosomal abnormalities in human chromosome 3p22.1 and/or 10q22-23 on the same single cell previously identified by the morphological stain. Such an analysis enables determination of presence or absence of genetic abnormalities in specific cells of a sputum sample which are identified by the morphological stain such as lower airway tract cells, lung cells or cytologically abnormal cells.

Thus, according to one aspect of the invention there is provided a method of identifying a genetically abnormal cell in a sputum sample. The method is effected by (a) staining a sputum sample using a morphological stain so as to identify a lower airway tract cell or lung cell in the sputum sample; and (b) staining the sputum sample using fluorescent in situ hybridization (FISH) so as to identify in the lower airway tract cell or lung cell a genetic abnormality in at least one of human chromosome 3p22.1 and 10q22-23, thereby identifying the genetically abnormal cell in the sputum sample.

As used herein the phrase “sputum sample” refers to a biological sample expectorated from the respiratory tract excluding that from the nasal passages, and includes mucus or phlegm mixed with saliva which can then be spat from the mouth. The sputum sample typically includes cells of the lower respiratory tract such as of the lungs, bronchi, alveoli, as well as cells of the upper respiratory tract such as of the trachea, larynx, pharynx and mouth.

The sputum sample can be collected from the subject by coughing and/or spitting into a collecting container (a non-induced sputum sample). Additionally or alternatively, the sputum sample can be collected from the subject after the subject inhales a substance (e.g., saline) which induces a more deep cough and results in the accumulation of more cells of the lower airway tract and lungs in the collecting container (an induced sputum sample).

Prior to staining, the sputum sample can be treated so as to isolate cells contained therein. For example, the sputum sample can be treated with dithiothreitol (DTT) and dithioerythritol which break the disulphide bonds in the mucin molecules, and release the cells from the mucus. To remove mucus and other debris from the cells, the sputum sample is further subjected to filtration e.g., using a nylon mesh. Following filtration, the isolated cells can be placed on microscopic slides e.g., using a cyto-centrifuge or as cell smears.

The genetic abnormality can be for example chromosomal aneuploidy (i.e., when the number of chromosomes is not diploid) such as a complete or partial multisomy [i.e., excess of chromosomes, e.g., trisomy (three copies of a certain chromosome) or polysomy (at least three copies of more than one chromosome)] and monosomy (presence of only one copy of a certain chromosome), imbalanced rearrangement such as imbalanced translocation or imbalanced inversion, inversion, deletion, macrodeletion, microdeletion and/or complete or partial chromosomal duplication.

As used herein the phrase “morphological stain” refers to a dye or a combination of dyes which enables visualization of a cell's morphology. Non-limiting examples of morphological stains which can be used by the method of the invention include May-Grünwald-Giemsa stain, Giemsa stain, Diff-Quick stain, Papanicolaou stain, Hematoxylin-Eosin stain, and DAPI stain.

According to some embodiments of the invention, the morphological stain used by the method of the invention is May-Grünwald-Giemsa stain, Giemsa stain, Diff-Quick stain, Papanicolaou stain or Hematoxylin-Eosin stain. Morphological stains are readily available from Sigma (Sigma, St Louis, Mo., USA), and Merck (KGaA, Darmstadt, Germany).

Following staining with the morphological stain, the cells are viewed via a microscope or an imaging device. As mentioned, once the cell morphology is visualized, the cell can be identified based on its morphological characteristics defining the cell type, source (e.g., the tissue of origin), developmental stage and/or malignant/normal state (e.g., pre-malignant, malignant or normal). Identification of cells of a sputum sample can be done by anyone skilled in the art of histology or pathology based on the known morphological features of each cell type as described in any histological text book which includes description of the respiratory tract and lungs [see e.g., Hypertext Transfer Protocol://World Wide Web (dot) lab (dot) anhb (dot) uwa (dot) edu (dot) au/mb140/CorePages/Respiratory/Respir (dot) htm#LARYNX; and Hypertext Transfer Protocol://World Wide Web (dot) med-ed (dot) virginia (dot) edu/public/CourseSitesDocs/CellandTissueStructure/handouts/unrestricted/original/MM Hndt_Respiratory (dot) html] and as briefly exemplified herein and in Example 2 of the Examples section which follows.

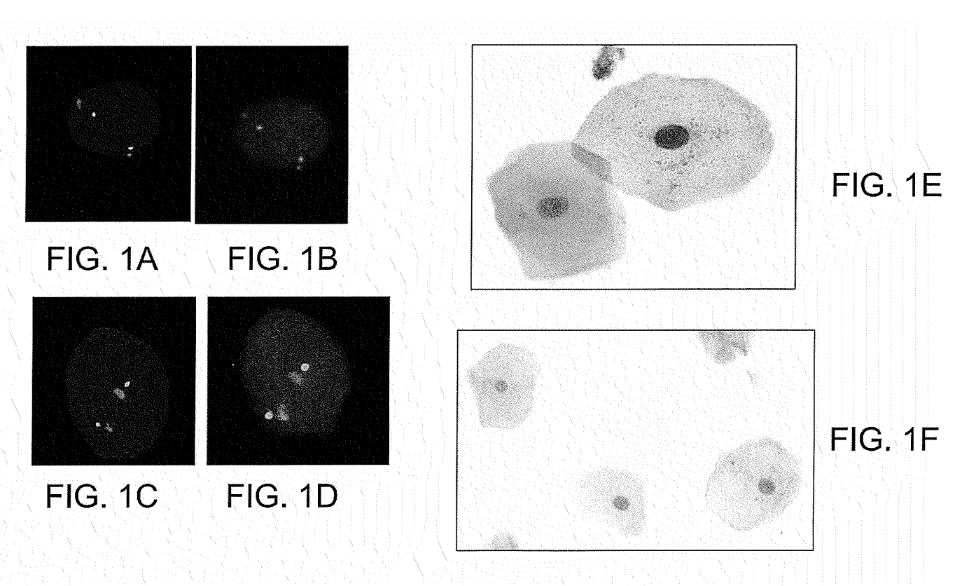

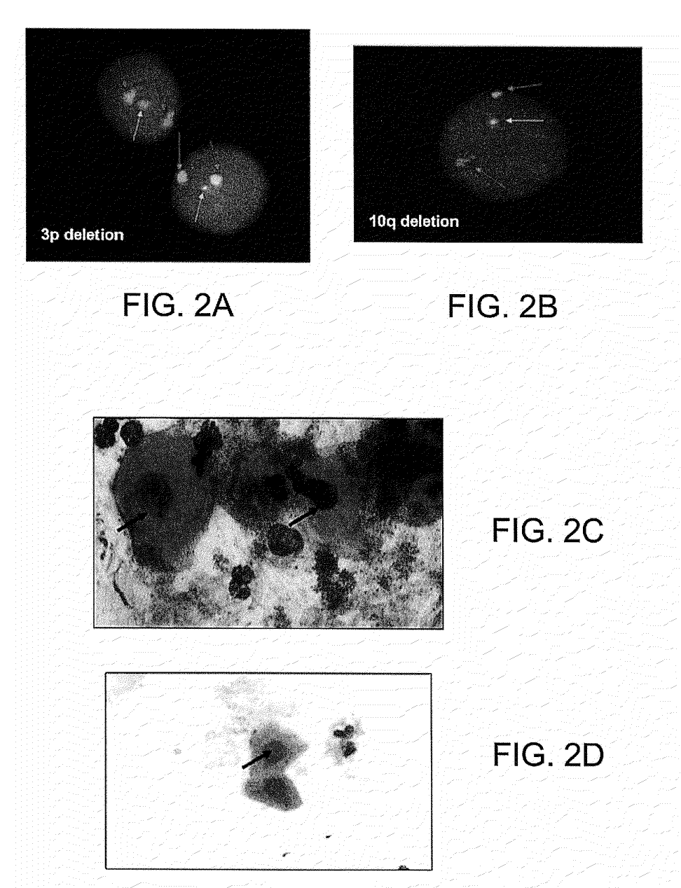

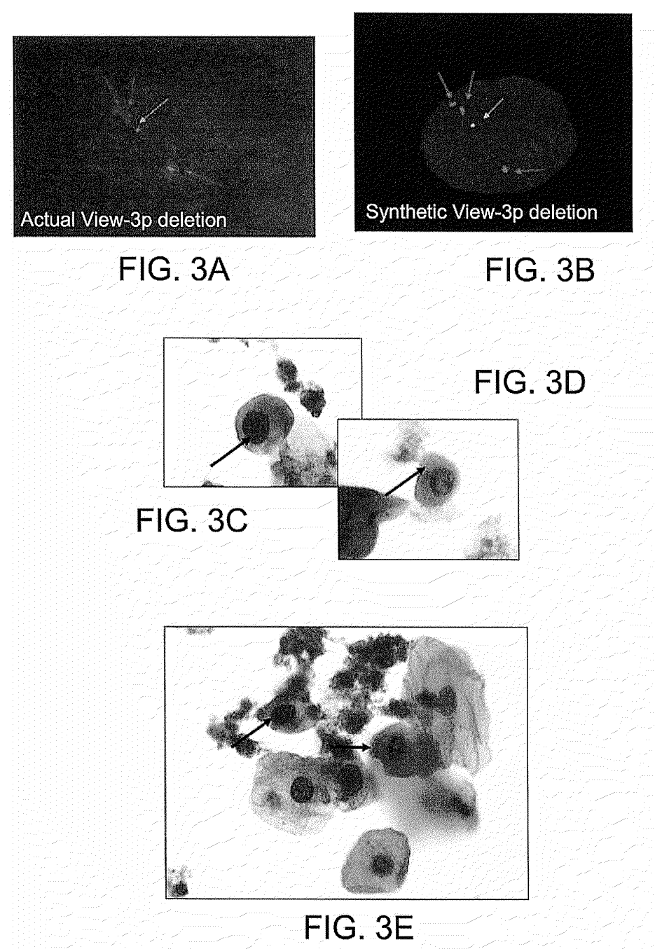

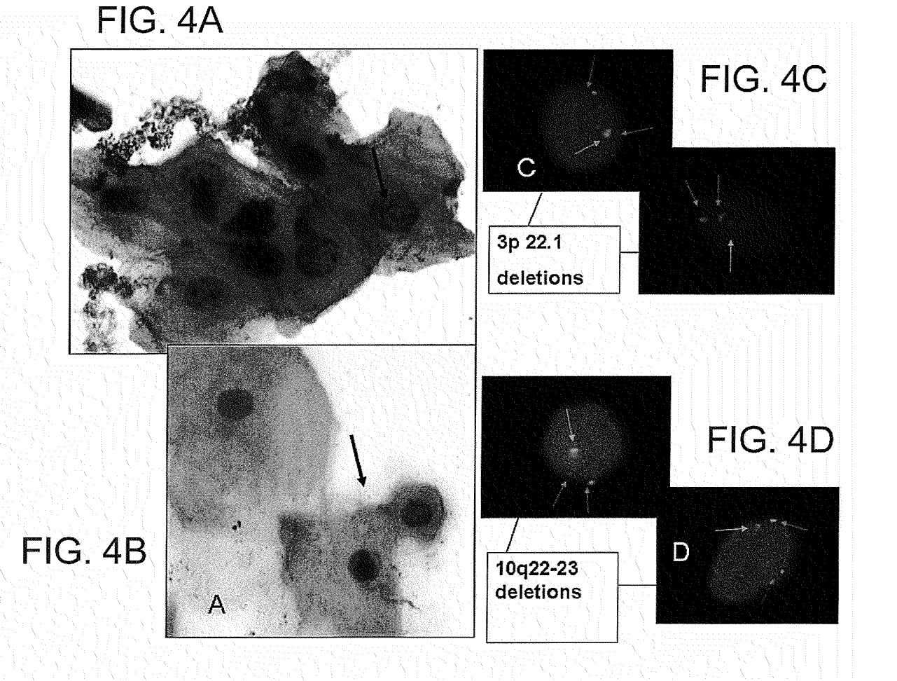

A sputum sample may include cells of the a lower airway tract (lower respiratory tract) including respiratory epithelial cells such as goblet cells, ciliated cells and non-ciliated cells (also called Clara cells, present also in bronchium), lung cells [epithelial cells of the alveoli (alveolar type I and type II cells) and alveolar macrophages], cells of the upper respiratory tract, trachea, pharynx, larynx and mouth, such as squamous epithelial cells, and blood cells [red blood cells (RBC) and white blood cells [such as lymphocytes, polymorphonuclear (PMN) cells, and other white blood cells (WBC)]. It should be noted that a sputum sample may also include abnormal cells, such as cells derived from the lower airway tract and lungs which underwent morphological changes (including reversible and un-reversible change). Non-limiting examples of morphologically abnormal cells of the lower airway tract or lung include squamous metaplasia cells, squamous atypia and squamous dysplasia cells. Example 2 of the Examples section which follows and FIGS. 1E-F, 2C-D, 3C-E, 4A-B, 7A-J, 8A, C and E, 10A-E, 11A-F, 13A-B, 14 and 15 provide description and images of the morphological characteristics of the cells which may be present in the sputum sample.

The phrase “lower airway tract cell or lung cell” as used herein encompasses morphologically normal and morphologically abnormal cells of the lower airway tract and lung. Examples of morphologically normal lower airway tract cells include respiratory epithelial cells such as goblet cells, ciliated cells and non-ciliated cells; examples of morphologically normal lung cells include epithelial cells of the alveoli such as alveolar type I and type II cells and alveolar macrophages; and examples of morphologically abnormal cells of the lower airway tract or lung include squamous metaplasia cells, squamous atypia and squamous dysplasia cells.

According to the method of this aspect of the invention, the morphological stain enables the identification of a lower airway tract cell (e.g., a goblet cell, a ciliated cell, a non-ciliated cell) and a lung cell (e.g., an alveolar type I cell, an alveolar type II cell and an alveolar macrophage) as well as of lower airway tract cells and lung cells having morphological abnormalities (e.g., squamous metaplasia cells, squamous atypia and squamous dysplasia cells).

According to some embodiments of the invention, cells of a sputum sample which are excluded from the subsequent FISH analysis (i.e., not selected for the subsequent FISH scan based on the morphological staining) are those derived from the upper airway tract, upper part of the trachea, pharynx, larynx and mouth such as squamous epithelial cells (see Example 2 of the Examples section which follows).

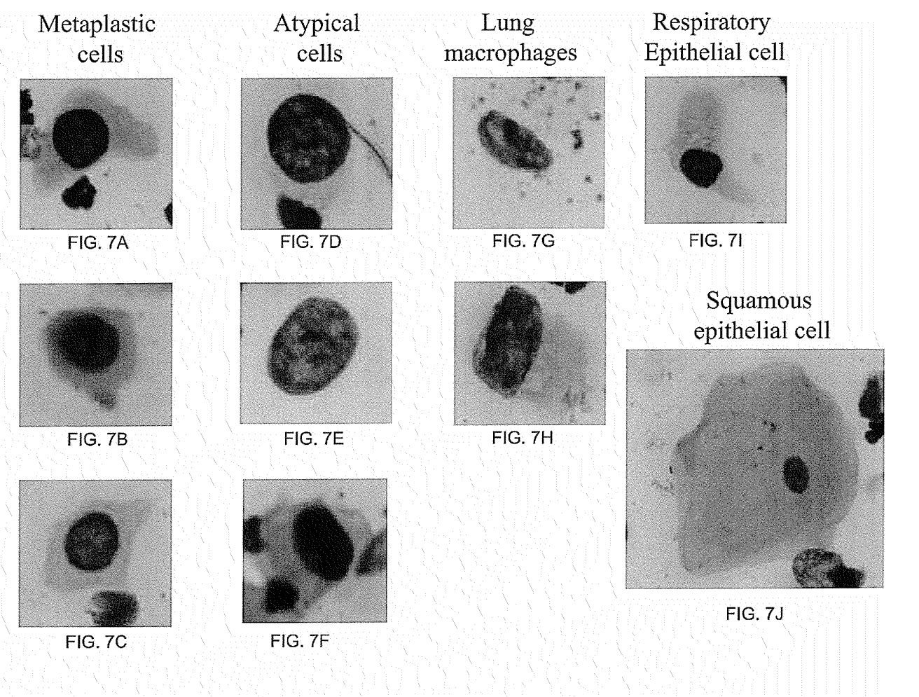

A squamous epithelial cell is a large, very flat cell, with irregular shape (not round, not oval) and characterized by a small nucleus/cytoplasm ratio. FIGS. 1E, 1F and 7J depict exemplary images of squamous epithelial cells.

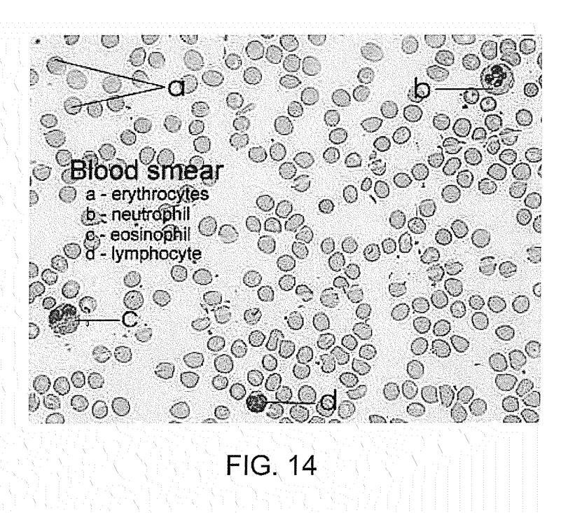

According to some embodiments of the invention, cells which are excluded from the subsequent FISH analysis (based on the morphological staining) are blood cells (See Example 2 of the Examples section which follows and FIG. 14).

Once identified, images of the stained lower airway tract cell or lung cell can be stored and the position (i.e., coordinate location) of the cell-of-interest on the slide is saved for a later reference when evaluating FISH results (signals) on the same single cell.

According to some embodiments of the present invention, prior to FISH staining, the morphologically stained cells are subjected to destaining (removal of the previous morphological stain from the cells) to prevent imaging interference of residual morphological stain with the subsequent FISH stain. Methods of destaining are well known in the art and are provided in the Examples section which follows.

As used herein the phrase “fluorescent in situ hybridization (FISH)” refers to a fluorescent method of detecting a presence or absence, order and/or a copy number of a nucleic acid sequence in a chromosomal DNA sample.

FISH is typically performed using at least one FISH probe. As used herein the phrase “FISH probe” refers to a labeled isolated polynucleotide having a nucleic acid sequence hybridizable to a target chromosomal DNA sequence. The target chromosomal DNA sequence can be a specific locus or gene on the chromosome, several loci or genes, a centromeric region of a chromosome, a repetitive sequence of a chromosome, satellite sequences, or the complete chromosome.

The FISH probe can be in a form of a plasmid, a bacteriophage, a yeast artificial chromosome (YAC) or a bacterial artificial chromosome (BAC). The length of the FISH probe can be selected such that it produces a detectable signal upon binding with the target chromosomal DNA, yet with high specificity to the sequence of interest (e.g., the specific gene or locus of interest). For example, the FISH probe can be of at least 1500 nucleotides and yet results in a specific FISH signal (see Knoll, J. H. M., Methods in Molecular Biology, 2007, 374: 55-66). A FISH probe can be of 1000-2000 bases (see e.g., WO00212563), or longer, such as at least 2 kilobases.

The FISH probe can be either directly labeled by conjugating a fluorophore via a linker or a chemical bond to at least one nucleotide of the probe, or indirectly labeled, by conjugating a non-labeled moiety which is bindable to a fluorescently-labeled counterpart. Non-limiting examples of suitable binding counterparts include biotin and streptavidin; biotin and avidin; an enzyme (e.g., Horse Radish Peroxidase) and a substrate (e.g., o-phenylenediamine); digoxigenin and an anti-digoxigenin antibody.

As used herein the term “fluorophore” refers to an entity which can be excited by light to emit fluorescence. Such a fluorphore can be an artificial or a naturally occurring molecule [e.g., fluorescein, eosin, an acridine dye, Texas Red, rhodamine, TAMRA, AMCA, TRITC, FITC, Cy2, Cy3, Cy5, Cy7, 6-FAM, HEX, 6-JOE, Oregon green 488, Oregon green 500, Oregon green 514, pacific blue, REG, ROX, TET, Alexa 350, Alexa 430, BODIPY 630/650, cascade blue, AlexaFluor P568, AlexaFluor P546, AlexaFluor P660, Spectrum ORANGE, Spectrum AQUA, Spectrum GREEN, Spectrum RED and the like), or a quantum dot. Quantum dots are coated nanocrystals fabricated from semiconductor materials in which the emission spectrum is controlled by the nanocrystal size. Quantum dots have a wide absorption spectrum, allowing simultaneous emission of fluorescence of various colors with a single excitation source. Quantum dots can be modified with large number of small molecules and linker groups such as conjugation of amino (PEG) or carboxyl quantum dots to streptavidin (Quantum Dot Corporation, Hayward, Calif., USA).

Suitable FISH probes can be identified by searching available databases such as the National Center for Biotechnology Information (NCBI) registry (Hypertext Transfer Protocol://World Wide Web (dot) ncbi (dot) nlm (dot) nih (dot) gov/projects/genome/clone/] and are available in either a labeled or an unlabeled form from suppliers such as Vysis (Downers Grove, Ill.), Abbot (Des Plaines, Ill.), and Invitrogen Corp., Carlsbad, Calif.

Non-limiting examples of suitable FISH probes which can detect a chromosomal aberration in the human 3p22.1 locus include the BAC clone for 3p22.1 (RP11-391M1; Invitrogen Corp., Carlsbad, Calif.) as set forth by GenBank Accession No. AC104186 (SEQ ID NO:1); a polynucleotide which comprises the RPL14 gene (GenBank Accession No. for transcript—NM_{—}003973; Genomic sequence—nucleotides 40473805-40478863 of GenBank accession No. NC_{—}000003.10 (SEQ ID NO:2), a polynucleotide which comprises the ENTPD3 gene (CD39L3; nucleotides 40403694-40445114 of GenBank Accession No. NC_{—}000003.10 (SEQ ID NO:3); GenBank Accession No. NM_{—}001248.1 for transcript); a polynucleotide which comprises the GC20 gene [nucleotides 40326177-40328919 of GenBank Accession No. NC_{—}000003.10 (SEQ ID NO:4)] and PMGM (CADM2; nucleotides 85858322-86200641 of GenBank Accession No. NC_{—}000003.10 (SEQ ID NO:5)]. Additional suitable FISH probes which can identify a genetic abnormality in the human chromosome 3p22.1 are provided in Table 24 in Example 5 of the Examples section which follows.

Non-limiting examples of suitable FISH probes which can detect a chromosomal aberration in the human 10q22.2-q23.1 locus include the BAC clone for 10q22.2-q23.1 (clone RP11-506M13; Invitrogen Corp., Carlsbad, Calif.) as set forth in GenBank Accession No. AC068139 (SEQ ID NO:6); a polynucleotide which comprises the SP-A which comprise SFTPA1 [nucleotides 81040722-81045206 on GenBank Accession No. NC_{—}000010.9 (SEQ ID NO:7)] and SFTPA2 [locus tag RP11-589B3.4, nucleotides 80990169-80985614 on GenBank Accession No. NC_{—}000010.9 (SEQ ID NO:8)]; a polynucleotide which comprises the PTEN/MMAC1 [phosphatase and tensin homolog, GenBank Accession AF067844; nucleotides 89613175-89718512 on GenBank Accession No. NC_{—}000010.9 (SEQ ID NO:9)]. Additional suitable FISH probes which can identify a genetic abnormality in the human chromosome 10q22.2-q23.1 are provided in Table 25 in Example 5 of the Examples section which follows.

Further description of suitable FISH probes is provided in WO00212563 A2 to Katz Ruth et al., which is fully incorporated herein by reference.

It will be appreciated that detection of specific deletions may require a combination of a locus-specific FISH probe with a chromosomal-specific FISH probe, derived from chromosome 3 or 10, which indicates the copy number of the respective chromosome (i.e., human chromosome 3 or 10). Such a FISH probe can be derived from the centromere of chromosome 3 or 10 (i.e., centromeric-specific FISH probe) or from any known sequence of the chromosome. Non-limiting examples of chromosomal-specific FISH probe which can be used along with the 3p22.1 or the 10q22.2-q23.1 locus-specific FISH probes include: Chromosome 3 alpha satellite probes or centromeric-specific FISH probe (from various manufacturers) and BCL6 (for Chromosome 3); and Chromosome 10 alpha satellite probes or centromeric-specific FISH probe (from various manufacturers) and PTEN (for chromosome 10).

For example, identification of chromosomal aberrations on human chromosome 3p22.1 can be performed using a locus-specific FISH probe (a FISH probe specific to human chromosome 3p22.1) and a centromeric-specific FISH probe (e.g., CEP 3). In addition, identification of chromosomal aberrations on human chromosome 10q22-23 can be performed using a locus-specific FISH probe (a FISH probe specific to human chromosome 10q22-23) and a centromeric-specific FISH probe (e.g., CEP 10). Additionally or alternatively, identification of chromosomal aberrations on human chromosome 10q22-23 and 3p22.1 can be performed using two locus-specific FISH probes (a FISH probe specific to human chromosome 10q22-23, and a FISH probe specific to human chromosome 3p22.1).

According to some embodiments of the invention, FISH is effected using at least three FISH probes. For example, FISH can be performed using a FISH probe specific to human chromosome 3p22.1, a FISH probe specific to human chromosome 10q22-23 and a FISH probe specific to a centromeric region of human chromosome 3 (e.g., CEP 3). Additionally or alternatively, FISH can be performed using a FISH probe specific to human chromosome 3p22.1, a FISH probe specific to human chromosome 10q22-23 and a FISH probe specific to a centromeric region of human chromosome 10 (e.g., CEP 10).

According to some embodiments of the invention, FISH is effected using at least four FISH probes. For example, FISH can be performed using a FISH probe specific to human chromosome 3p22.1, a FISH probe specific to human chromosome 10q22-23, a FISH probe specific to a centromeric region of human chromosome 3 (e.g., CEP 3) and a FISH probe specific to a centromeric region of human chromosome 10 (e.g., CEP 10).

Methods of employing FISH analysis on interphase chromosomes are known in the art. Following is a non-limiting example of FISH hybridization and after-hybridization wash conditions which can be used by the method of the invention. Directly-labeled probes [e.g., the RP11-506MI3 FISH probe specific to human chromosomal locus 10q22.2-q23.1 labeled with Spectrum Green dUTP or with Spectrum Red dUTP (Vysis), the RP11-391M1 FISH probe specific to the 3p22.1 locus labeled with Spectrum Green dUTP, CEP 3 FISH probe specific to centromere chromosome 3 labeled with Spectrum Orange; and CEP 10 FISH probe specific to centromere chromosome 10 labeled with Spectrum Orange or Spectrum aqua] are mixed with hybridization buffer (e.g., LSI hybridization buffer, Vysis) and a carrier DNA (e.g., human Cot-1 DNA, available from Life Technologies, Rockville, Md.). The probe solution is applied on microscopic slides containing the sputum samples and the slides are covered using a coverslip. The probe and the cells on the slides are co-denatured, e.g., for 3 minutes at 70° C. and are further incubated for hybridization e.g., for overnight incubation at 37° C. using an hybridization apparatus (e.g., HYBrite, Abbott Cat. No. 2J11-04). Following hybridization, the slides are washed to remove excess of unbound labeled probe and/or non-specific binding [e.g., 2 minutes at 72° C. in a solution of 0.3% NP-40 (Abbott) in 60 mM NaCl and 6 mM NaCltrate (0.4×SSC)], followed by a wash of 1 minute in a solution of 0.1% NP-40 in 2×SSC at room temperature], following which the slides are counterstained. Counterstaining is performed using, for example, 4′,6-diamidino-2-phenylindole (DAPI) and evaluated under a fluorescence microscope equipped with the appropriate filter combinations. If the hybridization signals were deemed satisfactory, the slides are sent for automated FISH scanning.

Following FISH, cells-of-interest are imaged using a fluorescent microscopy mode with appropriate filters. Thereafter, images of cells of interest stained in a morphological stain and in FISH stain can be viewed. According to some embodiments of the invention, imaging of the cells is performed using at least two imaging modalities, e.g., a bright field modality for viewing the morphological stain and a dark field modality for viewing the FISH stain. Imaging can be effected simultaneously by viewing at the same time the stored images of the same single cell stained with the morphological stain (one image) and the FISH stain (another image). Alternatively, imaging can be effected sequentially by viewing the cells following the morphological stain, selecting cells-of-interest for the subsequent scan and viewing the selected cells-of-interest after the FISH stain.

According to some embodiments of the invention, imaging is effected using a device capable of dual imaging, i.e., a bright field and a dark field imaging modes. Such a device can be an automated image analysis device capable of at least dual imaging. A suitable imaging apparatus which can be used for executing the method of the invention is the Bio View Duet™ (Bio View, Rehovot, Israel).

The above teachings can be efficiently harnessed to the clinical evaluation of cytological samples for the diagnosis of lung cancer. The method is based on the identification in a sputum sample of genetically abnormal lower airway tract/lung cells (having chromosomal aberrations in the human chromosome 3p22.1 and/or 10q22-23 loci) above a predetermined threshold (in absolute number or percentage). Thus, as shown in the Examples section which follows, the novel method of the invention resulted in unprecedented sensitivity and specificity values of lung cancer diagnosis such as a sensitivity of 93.1% and a specificity of 80.95% (Table 23, Example 4). This is in sharp contrast to the sensitivity and specificity values which are obtained when using other non-invasive methods of diagnosing lung cancer, such as sputum cytology alone which results in 31.4% sensitivity and 87% specificity when considering moderate and severe dysplasia as predictors of the presence of lung cancer (Example 1), or in 11.4% sensitivity and 93.5% specificity when considering severe dysplasia as a predictor for lung cancer (Example 1); or FISH scan alone which results in 81.8% sensitivity and 80% specificity (Example 3).

Thus, according to an aspect of some embodiments of the invention, there is provided a method of diagnosing of lung cancer in a subject. The method is effected by (a) staining a sputum sample of the subject with a morphological stain so as to identify lower airway tract cells or lung cells in the sputum sample; and (b) staining the sputum sample with FISH so as to identify a genetic abnormality in at least one of human chromosome 3p22.1 and 10q22-23 in the lower airway tract cells or lung cells identified in step (a), wherein a percentage or number above a predetermined threshold of the lower airway tract cells or lung cells having the genetic abnormality is indicative of the lung cancer, thereby diagnosing the lung cancer in the subject.

The term “diagnosing” as used herein refers to determining presence or absence of a disease, classifying a disease severity or symptom, monitoring disease progression, forecasting an outcome of a disease and/or prospects of recovery.

The phrase “lung cancer” as used herein encompasses small cell lung cancer, non-small cell lung cancer (NSCLC) and metastatic lung cancer (i.e., a cancer comprising cancerous cells originating in a distant organ and penetrating into the lung tissue). Non-limiting examples of cancerous cells which can form cancer metastasis in the lung tissue include breast cancer, colon cancer, prostate cancer, sarcoma, bladder cancer, neuroblastoma and Wilm's tumor).

As used herein the term “subject” refers to a human being who may be of any age or gender.

According to some embodiments of the invention, the subject is at a risk of developing lung cancer due to genetic, environmental and/or occupational hazard factors. Non-limiting examples of known risk factors include tobacco or Marijuana smoking; exposure to asbestos, radon, radioactive ores such as uranium, chemicals such as arsenic, vinyl chloride, nickel chromates, coal products, mustard gas and chloromethyl ethers, industrial grade Talcum powder (which may contain asbestos); recurring inflammation (e.g., Tuberculosis, pneumonia); personal and family history; vitamin A deficiency or excess; and air pollution.

According to some embodiments of the invention, morphology-stained cells are scanned under Bright field illumination (morphology scan) to identify cells-of-interest [i.e., cell exhibiting morphological characteristics typical to pre-defined cells such as lower airway tract cells or a lung cells (see e.g., FIGS. 7A-I; and Example 2)] and to exclude squamous epithelial cells (see e.g., FIGS. 1E, 1F and 7J) and blood cells (see FIG. 14) from the analysis. During the morphology scan, images are produced for all cells and their coordinates are saved. Cells-of-interest (lower airway tract and lung cells) are classified automatically or manually into a sub-group (target cells), and are further subjected to evaluation of FISH results, thus identifying the genetic abnormalities in human chromosome 3p22.1 and/or 10q22-23 on the same target cells identified by the morphological scan.

The predetermined threshold can be determined experimentally by comparing two groups of individuals, one group includes subjects diagnosed with lung cancer and another group includes healthy controls (free of the disease), essentially as described in Examples 3 and 4 of the examples section which follows.

According to some embodiments of the invention, the threshold is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25% of genetically abnormal cells (having chromosomal aberrations in human chromosome 3p22.1 and/or 10q22-23) out of the identified lower airway tract cells or lung cells in the sputum sample. It should noted that in many cases, a sputum sample of a subject with lung cancer may include more than 50% of genetically abnormal cells out of the total identified nucleated lower airway tract cells or lung cells.

According to some embodiments of the invention, the threshold is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, e.g., at least 50, at least 75, at least 100, at least 120, at least 150 genetically abnormal cells (having chromosomal aberrations in human chromosome 3p22.1 and/or 10q22-23) of the identified lower airway tract cells or lung cells in the sputum sample.

As shown in Example 4, the threshold can vary dependant on the minimal number of lower airway tract cells or lung cells identified by the morphological staining. For example, in samples containing less than 200 target lower airway tract cells or lung cells, the threshold is above 10% (i.e., at least 10% of the cells-of-interest should exhibit genetic abnormalities in human chromosome 3p22.1 and/or 10q22-23). Similarly, in samples containing between 200 and 1000 target lower airway tract cells or lung cells, the threshold is above 7.5%; and in samples containing more than 1000 target lower airway tract cells or lung cells, the threshold is above 5%.

It should be noted that the sensitivity and specificity of the method can vary depending on the parameters (chromosomal aberrations) used for the “target scan” (i.e., analyzing FISH signals on morphologically identified cells-of-interest which exclude squamous epithelial cells and blood cells present in a sputum sample) and the number of qualified cells-of-interest. For example, as shown in Table 13 (Example 3), when the “Total 3p (−Abn.)/3p # targets” parameter was used [i.e., the sum of “3p # del” (cells showing 2 centromeric signals of chromosome 3 and one green signal at 3p) and “3p # Poly” (cells showing >2 centromeric signals of chromosome 3 and >2 green signals of 3p locus) divided by “3p # targets” (the number of total relevant cells scored using the 3p and 3 cen probes)] and the minimal number of cells-of-interest was higher than 50, a presence of more than 0.04 (i.e., 4%) of genetically abnormal cells was indicative of the diagnosis of lung cancer with a sensitivity of 91.7% and a specificity of ≧80%. On the other hand, when samples which include less than 50 cells were included and scored using the same parameter, the sensitivity was 80% and the cutoff for positive diagnosis was 0.05 (Table 13).

While further reducing the present invention to practice, the present inventors have uncovered that a higher sensitivity of lung cancer diagnosis can be achieved when cells of a sputum sample are analyzed according to more than one parameter of chromosomal aberrations, such as two parameters of a target scan (see e.g., Table 17, Example 3), or a combination of parameters of “target scan” (as described above) with an “area scan” (analyzing FISH signals on all cell types present in the sputum sample, regardless of their morphological characteristics). For example, as shown in Table 17 (Example 3), using a “target scan” with the “Total 3p (−Abn.)” parameter [i.e., the sum of “3p # del” (cells showing 2 centromeric signals of chromosome 3 and one signal at 3p) and “3p # Poly” (cells showing >2 centromeric signals of chromosome 3 and >2 signals of 3p locus)] with a cutoff of 6 (i.e., a positive diagnosis of lung cancer is made if at least 6 cells of the analyzed cells by the target scan exhibit these chromosomal aberrations) and an “area scan” with the “Total 10q” parameter [i.e., sum of genetic abnormality for chromosome 10 found (10q # del, 10q # Abn, 10q # Poly)] with a cutoff of 1.18% (i.e., a positive diagnosis of lung cancer is made if at least 1.18% of the analyzed cells by the area scan exhibit these chromosomal aberrations), resulted 100% sensitivity and >80% specificity. Thus, a subject is diagnosed with lung cancer if cells of the sputum sample exhibit chromosomal aberrations above the respective cutoffs of either of the two parameters (e.g., an area scan parameter and a target scan parameter).

Thus, according to an aspect of some embodiment of the invention the method of diagnosing lung cancer is effected by (a) staining a sputum sample with a morphological stain so as to identify lower airway tract cells or lung cells in the sputum sample; (b) staining the sputum sample with FISH so as to identify a genetic abnormality in at least one of human chromosome 3p22.1 and 10q22-23 in cells of the sputum sample, wherein a percentage or number above a predetermined threshold of: (i) the lower airway tract cells or lung cells of the sputum sample identified in step (a) having the genetic abnormality; or (ii) the cells of the sputum sample having the genetic abnormality; is indicative of the lung cancer, thereby diagnosing the lung cancer in the subject.

According to some embodiments of the invention the adequacy of the sputum sample for FISH analysis is determined by the presence of at least 50 cells of the lower airway tract and/or lungs (as identified by the morphological stain) in the sputum sample.

A sputum sample that meets the adequacy criteria can be further analyzed for presence of genetic abnormalities in cells of the lower airway tract or lungs of the sputum sample; and/or for the presence of genetic abnormalities in cells of the sputum regardless of the cell's morphology.

According to some embodiments of the invention, the threshold of the percentage of cells of the sputum sample (all types of cells, regardless their morphology) having the genetic abnormality is at least about 0.16%, at least about 0.18%, at least about 0.20%, at least about 0.22%, at least about 0.24%, at least about 0.26%, at least about 0.28%, at least about 0.30%, at least about 0.35%, at least about 0.40%, at least about 0.45%, at least about 0.50%, at least about 0.55%, at least about 0.60%, at least about 0.65%, at least about 0.70% (e.g., 0.74), at least about 0.75%, at least about 0.80%, at least about 0.85%, at least about 0.90%, at least about 0.95%, at least about 1%, at least about 1.1% (e.g., 1.15%, 1.18%), at least about 1.2%, at least about 1.3%, at least about 1.6%, at least about 2% (e.g., 2.25%), at least about 3%, at least about 3.5% (e.g., 3.75%), at least about 4% (e.g., 4.12%), at least about 5%, at least about 7%, at least about 10%, at least about 20% of the cells of the sputum sample.

Thus, according to the method of this aspect of the invention, a subject is diagnosed with lung cancer if at least one of the above following two criteria is met, i.e., if the sputum sample includes a percentage or number above a predetermined threshold of lower airway tract cells or lung cells identified by the morphological stain (i.e., classified as lower airway tract cells or lung cells based on the morphological characteristics shown after staining with the morphological stain) and having the genetic abnormality (based on the FISH analysis on the morphologically-identified cells); or if the sputum sample includes a percentage or number of cells above a predetermined threshold [all types of cells, regardless of their morphology or origin, e.g., cells of the lower (e.g., squamous epithelial cells) and upper airway tract and/or blood cells] having the genetic abnormality (based on FISH analysis alone).

According to some embodiments of the invention, the FISH analysis performed on cells of the sputum sample (regardless of their morphology) can be effected on a different cell sample (smear of sputum cells or cytocentrifuged sputum cells) of the same sputum sample that was found to be adequate (as described above).

Following is a non-limiting example of the method of diagnosing lung cancer according to the present teachings. A sputum sample is stained with a morphological stain, following which the stained cells are identified based on their morphological characteristics and classified to “target cells” which include lower airway tract cells and lung cells, including cytologically normal and abnormal cells of the lower airway tract or lung origin; or to “non-target cells” which are excluded from the target scan such as squamous epithelial cells or blood cells. The cell coordinates of the target cells are saved for the subsequent target FISH analysis. The cells are destained to remove the morphological stain, and further subjected to FISH analysis to detect chromosomal aberrations in human chromosome 3p22.1 and/or 10q22-23. Following FISH analysis cells with a genetic abnormality in human chromosome 3p22.1 and/or 10q22-23 are identified, regardless of their morphological classification (i.e., all cell types present in the analyzed area), and the percentage of cells having the genetic abnormality is determined (area scan result). Next, FISH analysis is performed on the target cells that were identified by the morphology stain as lower airway tract or lung cells (normal and abnormal cells of the lower airway tract or lung tissue) according to the saved coordinates of these cells. The percentage or number of target cells having the genetic abnormality out of the total identified target cells is determined (target scan result). A diagnosis of lung cancer is made if the result of target scan and/or the area scan is above a predetermined threshold (the respective cutoff for each analysis).

The morphological stain and the FISH probe specific for human chromosome 3p22.1 and/or 10q22-23 which are described hereinabove for detecting genetically abnormal lower airway tract cells or lung cells and diagnosing lung cancer may be included in a diagnostic kit/article of manufacture preferably along with appropriate instructions for use in detecting genetically abnormal cells and/or diagnosing lung cancer and labels indicating FDA approval for such use(s).

According to some embodiments of the invention, the instructions comprise a predetermined threshold of a percentage of genetically abnormal cells which is indicative of positive diagnosis of lung cancer.

Such a kit can include, for example, at least one container including the morphological stain, another container including the FISH probe or a mix of several FISH probes, and optionally also a detection reagent packed in third container (e.g., enzymes, secondary antibodies, buffers, chromogenic substrates, fluorogenic material). The kit may also include appropriate buffers and preservatives for improving the shelf-life of the kit.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Blue Histoloy, School of Anatomy and Human Biology, The University of Western Australia, Hypertext Transfer Protocol://World Wide Web (dot) lab (dot) anhb (dot) uwa (dot) edu (dot) au/mb140/CorePages/Respiratory/Respir (dot) htm#LARYNX; Gartner and Hiatt, COLOR TEXTBOOK OF HISTOLOGY, 2nd ed., pp. 343-364; Young and Heath, WHEATER'S FUNCTIONAL HISTOLOGY, 4th ed., pp. 222-236. Other general references are provided throughout this document. The procedures therein are believed to be well known in the art and are provided for the convenience of the reader. All the information contained therein is incorporated herein by reference.

### Example 1

### Valuation of the Diagnosis of Lung Cancer by Analyzing Parallel Sputa Specimens for Cytologic Atypia and Genetic Abnormalities

### Detection of Lung Cancer in Non-Induced Sputum

Detection of lung cancer by sputum cytology has low sensitivity but is noninvasive and, if improved, could be powerful for early lung cancer detection. The present inventors have tested whether the accuracy of diagnosing lung cancer by evaluating sputa for cytologic atypia and genetic abnormalities is greater than that of conventional cytology alone, as follows.

Materials and Experimental Methods

Study design and patient population—In this prospective clinical trial, the present inventors evaluated sputum samples collected from patients with lung cancer and from age-matched healthy (nonsmokers) or high-risk (history of heavy smoking) control subjects. None of the participants had received prior radiotherapy or chemotherapy. The University of Texas M.D. Anderson Cancer Center Investigational Review Board approved this study, and all study participants signed a consent form detailing the research methods. Trained staff interviewers from M.D. Anderson Cancer Center administered an epidemiologic questionnaire to all study participants. Data collected included demographic characteristics and history of tobacco use (9). In the cancer patients who underwent resection, sputum samples were collected before surgery. All high-risk smoker control subjects had helical CT scans negative for detection lung cancer at the time of study entry and for the following 2 years.

All participants (except for the healthy control subjects, who underwent induced sputum production after saline inhalation with a nebulizer) were instructed to cough into a container that was filled with Sacommano's fixative (90% alcohol, 5% acetic acid, and 5% polyethylene glycol) on 3 consecutive days on arising. Mailed in sputa were cytocentrifuged and filtered through gauze. The sediment was used to prepare at least 10 preparations. For FISH analysis, eight Cytospin preparations were generated on positively charged glass slides using a Shandon Cytospin 2 cytocentrifuge (Thermo Fisher Scientific, Inc., Waltham, Mass.) and fixed in a 4:1 mixture of methanol and acetone. The remaining two preparations were fixed in 95% alcohol for Papanicolaou's staining. Scoring of two Papanicolaou-stained sputa was performed by a cytotechnologist and two senior cytopathologists, none of whom had knowledge of the patients' clinical history. Discrepant diagnoses were scored by consensus over a multiheaded microscope by all three observers (C.A., N.P.C., and R.L.K.). Slides were screened and classified according to a seven-tiered scoring system as follows: negative, squamous metaplasia, mild dysplasia, moderate dysplasia, severe dysplasia, carcinoma, or insufficient for diagnosis. Slides were considered insufficient for diagnosis if they had zero to three histiocytes; excessive cellular degeneration; obscuring bacterial, fungal, or neutrophilic contamination; or limited numbers of epithelial cells.

In addition, some of the patients with lung cancer who submitted sputa provided subsequent tissue specimens at the time of surgery to resect the tumor as follows: Cytospin preparations of mainstem bronchial brushes on the side of the tumor [TBB] and the normal side contra-lateral to the tumor [NBB] taken just prior to surgery; touch preparations of resected lung cancer [TPP], adjacent normal bronchus [TAB], and distal normal lung tissue [NTP]. These specimens were sent for evaluation of centromeric chromosome 3, chromosome 3p22.1, centromeric chromosome 10, and chromosome 10q22-23, the same genetic markers that were evaluated in sputa. Before evaluation by FISH, touch preparations or Cytospin preparations from bronchial brushes, were evaluated for specimen adequacy and the presence or absence of malignant cells by Papanicolaou's stain. Evaluation of bronchial epithelial and tumor cells is described below.

FISH analysis—A two-color FISH assay using bacterial artificial chromosome (BAC) probes for 3p22.1 (GenBank Accession No. AC104186) labeled with SPECTRUM GREEN (Vysis) and 10q22-23 (GenBank Accession No. AC068139) labeled with SPECTRUM GREEN (Vysis), combined with commercial centromeric probes for chromosomes 3 (cep 3; Catalogue No. 06J36-003, Vysis, Inc., Downers Grove, Ill.) labeled with SPECTRUM ORANGE (Vysis) and chromosome 10 (cep 10; Catalogue No. 06J36-009, Vysis, Inc., Downers Grove, Ill.)) labeled with SPECTRUM ORANGE (Vysis) was performed on two separate slides (i.e., one slide with the 3p22.1 and centromere 3 probes and another slide with the 10q22-23 and centromere 10 probes).

The bacterial artificial chromosome clone located at 10q22.2-q23.1 (clone RP11-506MI3; Invitrogen Corp., Carlsbad, Calif.) includes about 180 kilo base pair (kb), and confirmed to contain genomic sequences of SP-A comprising both SFTPA1 (on 10q22.2-q23.1; surfactant protein A1B; also known as PSAP; PSPA; SFTP1; SFTPA1; MGC133365; AC068139.6) and SFTPA2 (on 10q22-q23; surfactant protein A2B; also known as SP-2A; SP-A1; SP-A2; SPAII; SFTPA2; AC068139.3), was isolated and labeled with Spectrum Green dUTP (Vysis) (5). The chromosomal location of the clone was confirmed on a normal metaphase spread in combination with a centromeric 10 probe directly fluorescence labeled with Spectrum Orange (Vysis).

The BAC clone for 3p22.1 (RP11-391M1), containing about 186 kb of genomic sequences and consisting of four known genes—RPL 14 (on 3p22-p21.2; ribosomal protein L14; also known as L14; RL14; hRL14; CTG-B33; MGC88594; CAG-ISL-7), ENTPD3 [on 3p21.3; ectonucleoside triphosphate diphosphohydrolase 3; also known as HB6; CD39L3; F1193839; NTPDase-3], GC20 [on 3p22.1; also known as eukaryotic translation initiation factor 1B (EIF1B); and translation factor sui1 homolog]—was obtained commercially from Invitrogen and grown in Escherichia coli. It was subsequently isolated, linearized, and labeled with Spectrum Green dUTP according to the manufacturer's directions (Vysis). Localization of the bacterial artificial chromosome clone on chromosome 3 was confirmed by using normal metaphase FISH. One hundred nanograms of each labeled probe was mixed with an equal quantity of human Cot-1 DNA (Life Technologies, Rockville, Md.) in 10 μl of LSI hybridization buffer (Vysis) and mounted on a slide together with 1 μl of either cep 3 (for 3p22.1) or cep 10 (for the SP-A gene on 10q22-23). Hybridization and post-washing were done as described previously (5). Counterstaining of nuclei was performed with 4′,6-diamidino-2-phenylindole and evaluated under a fluorescence microscope equipped with the appropriate filter combinations. If the hybridization signals were deemed satisfactory, the slides were sent for automated FISH scanning.

Automated FISH scanning—Slides were automatically scanned by the Duet™ (BioView Ltd. Rehovot, Israel). The Duet™ is based on a fully automated microscope (Olympus BX61, Japan), a motorized 8-slides stage (Marzhauser, Wetzler, Germany) and a 3CCD progressive scan color camera (JVC KYF75U, Japan). FISH scanning was performed using ×60 oil objective in fluorescent illumination and by using appropriate filters and a software program specifically designed to capture the orange and green fluorescent signals generated using the above described probes.

An average of 200 consecutively scanned cells (area scan) per subject were evaluated by two observers, blinded to the subjects' clinical status, using an automated scanning system and fluorescent microscope with custom software for scoring deletions or extra copies of chromosomes 3, 3p22.1, 10, 10q22-23 by FISH.

By the area scan the sputum samples were evaluated for FISH abnormalities. Only cells that were clearly non-overlapping and complete (e.g., non-ruptured), with well-preserved nuclei, and high-quality fluorescence signals without background fluorescence were scored. For each consecutive cell that was displayed on the video screen, the presence or absence of centromeric probes (orange signals) or locus-specific probes (green signals) relative to the centromere was recorded. No attempt was made to pre-select cells on the basis of nuclear size or shape. At the end of each scan, a pie chart displaying the level of chromosomal abnormalities for each category of aberrations was generated. Two experienced observers who were trained on the system and blinded to the patients' clinical status interactively confirmed the pie chart's classification using a series of filters from the screen or through the microscope within the system. In this way, the numbers of missing signals (deletions) and extra signals (polysomies) were confirmed. If discrepant with the automatically generated pie chart, the cells were reclassified. In addition, it was most important that “split” signals were not counted as two signals. Cells that could not be scored were discarded into an unclassified category.

The cells were scored as follows and expressed as percentages:

Deletions 3p: One green signal and two orange signals.
Monosomy cep3: One orange signal
Polysomy cep3: More then two orange signals
Polysomy 3p: More than two green signals
Deletions 10q: One green signal and two orange signals.
Monosomy cep10: One orange signal
Polysomy cep10: More then two orange signals
Polysomy 10q: More than two green signals
All Abnormalities 3: Sum all classifications of 3p and cep3.
Del 3p and Poly 3p: Sum deletions 3p and Polysomy 3p.
Aneusomy 3: Sum of monosomy cep3 and Polysomy cep3
All Abnormalities 10: Sum all classifications of 10q and cep10.
Del 10q and Poly 10q: Sum deletions 10q and Polysomy 10q.
Aneusomy 10: Sum of Monosomy cep10 and Polysomy cep10
All 3 abn and 10 abn: Sum all abnormalities for 3p, cep3, 10q and cep10.

Normal controls consisted of pooled human lymphocytes hybridized and quantitated automatically in the same batches as sputa for 3p22.1 and 10q22-23 as the mean number of cells with summed abnormalities for 3p22.1, cep 3, 10q22-23, and cep 10±1 standard deviation (SD).

For scoring FISH results from sites other than sputa (e.g., NBB, TBB, TPP, TAB, and NTP), a manual scoring system was used. From the bronchial brush specimens, TAB and NTP deletions of 3p22.1 or 10q22-23 relative to cep 3 or cep 10 were scored in 100 morphologically normal—appearing bronchial epithelial cells. In tumor touch preparations, tumor cells were evaluated for deletions of 3p22.1 and 10q22-23 relative to the internal centromeric probes. The accuracy of manual scoring was confirmed by a random sample check performed by a second cytogenetic technologist.

Papanicolaou stained sputum preparations—were evaluated by experienced cytopathologists for presence of cancer or cytological atypias according to a six tiered scheme (Franklin W A et al. WHO classification of tumors of the lung, pleura, thymus and heart, IARC press: lyon, 2004, pp. 68-72).

Statistical analyses—The mean, standard deviation, median, and range for continuous variables were analyzed using Wilcoxon's rank-sum test to assess for differences in the distribution of genetic abnormalities between cancer patients and control subjects. For categorical variables such as sex, smoking history, cytologic diagnosis, and disease stage, Fisher's exact test was used to assess the association between the different variables and cancer status.

Univariate and multivariate logistic regression models for estimating cancer status were also performed. From the multivariate models, receiver operator characteristic (ROC) curves were produced to estimate each individual's predicted probability of having cancer. In the model, all continuous variables were changed to categorical variables on the basis of their median separately, where the p value cutoff was chosen as 0.10. An ROC curve is a plot of the true-positive rate against the false-positive rate for the different possible cut points of estimated p value. An ideal prediction model will have 100% sensitivity (true positive) and 100% specificity (true negativity). The ROC curve captures the information about how good a prediction model is.

Correlations of genetic aberrations between epithelial cells from different specimens were performed using Spearman's rank correlation coefficient test.

Experimental Results

Patient population—A total of 71 subjects were enrolled in the trial, but five were excluded because they had poor-quality sputum specimens that did not produce evaluable cells for FISH analysis. Of the 66 subjects whose sputa could be evaluated, 35 had predominantly early-stage lung cancer and 31 were control patients, of whom 6 were healthy and 25 were at high risk for lung cancer because of their history of heavy smoking (see Table 1, hereinbelow). The patients with cancer had non-small cell lung cancer (NSCLC), classified predominantly as adenocarcinoma and squamous cell carcinoma. Most of the patients had peripherally based tumors.

TABLE 1
Characteristics of the subjects' populations

| | Cancer | No Cancer |
|---|---|---|
| | (N = 35) | (N = 31) |
| Sex | | |
| Female | 21 | 16 |
| Male | 14 | 15 |
| History of smoking (pack-year) | | |
| 0 | 7 | 6 |
| <20 | 7 | 1 |
| 20-50 | 12 | 16 |
| 50-100 | 6 | 5 |
| >100 | 2 | 1 |
| Age (median, range) | 65 (47-81) | 62 (27-75) |
| Age Mean | 65.1 | 59.2 |
| Stage | | |
| I/IA/IB | 19 | 0 |
| II | 7 | 0 |
| III/IIIA | 6 | 0 |
| IV | 2 | 0 |
| Unknown stage | 1 | |
| Location of Tumor | | |
| Central Tumor | 6 | 0 |
| Peripheral Tumor | 29 | 0 |
| Histology | | |
| Adenocarcinoma | 22 | 0 |
| Squamous Cell Carcinoma | 10 | 0 |
| Neuroendocrine Carcinoma | 2 | 0 |
| Non-small Cell Carcinoma | 1 | 0 |

TABLE 1. Provided are the characteristics of the subjects enrolled in the study, including smoking history (in terms of pack-year, i.e., a Pack Year equals the Number of packs smoked per day * Number of years as a smoker), and tumor's stage (according to Franklin et al staging system, Franklin WA et al. WHO classification of tumors of the lung, pleura, thymus and heart, IARC press: lyon, 2004, pp. 68-72), location and histological evaluation.

Of the patients with cancer with known stage, 26 patients were lower stage (stages I/IA/IB and II) and 8 were higher stages (stages III, IIIA and IV, Table 1). All patients had non-small cell carcinoma classified according to the WHO classification, predominantly adenocarcinoma and squamous carcinoma. Cytological diagnosis was strongly correlated to cancer status (Table 2). Cytological diagnoses of squamous metaplasia, or mild, moderate and severe dysplasia were significantly associated with cancer status.

TABLE 2
Association between cytologic diagnosis on
sputum and cancer status^{†}

| | Cytologic diagnosis | | | | | |
|---|---|---|---|---|---|---|
| | N (%) | | | | | |
| Cancer status | 1 | 2 | 3 | 4 | 5 | Total |
| Absent | 19 | 2 | 6 | 2 | 2 | 31 |
| | 70.37 | 16.67 | 50.00 | 22.22 | 33.33 | |
| Present | 8 | 10 | 6 | 7 | 4 | 35 |
| | 29.63 | 83.33 | 50.00 | 77.78 | 66.67 | |
| Total | 27 | 12 | 12 | 9 | 6 | 66 |

Table 2: Cytologic diagnosis: 1 = negative, 2 = squamous metaplasia, 3 = mild dysplasia, 4 = moderate dysplasia, and 5 = severe dysplasia.
^{†}p = 0.009 (Fisher's exact test).

Hybridization efficiency and cutoff values for FISH—Each locus-specific probe was confirmed on metaphases from normal lymphocytes to hybridize to the appropriate centromeric and locus-specific regions for cep 3, 3p22.1, cep 10, and 10q22-23. Cancer patients had an average of 183 cells (median, 131; range, 49-589) evaluated for 3p22.1 and 158 cells (median, 139; range, 35-455) evaluated for 10q22-23. Control subjects had an average of 204 cells evaluated for 3p22.1 (median, 183; range, 40-673) and 170 evaluated for 10q22-23 (median, 189; range, 13-474).

Similarly, diploid signals were noted for each probe in interphase nuclei from five batches of normal lymphocytes. The mean (±1 SD) in normal lymphocytes of the deletion and polysomy value for 10q22-23 was 1.14±0.59, and that for deletions and polysomies of cep 3 and 3p22.1 was 3.02±1.73. For all chromosomal abnormalities of 3 and 10, the mean and SD were 4.91±2.50.

Comparison of patient characteristics, genetic changes in sputum and other respiratory tract sites, tumor size, disease stage, and cancer status—There was no significant difference between cancer status for patients' age or smoking history in pack-years. There were, however, significant differences in the percentages of chromosomal abnormalities in epithelial cells in relationship to the patients' cancer status. Significantly more abnormalities in epithelial cells of 3p, deletions of 10q, all abnormalities of 3, deletions and polysomies of 3p, all abnormalities of 10, and all 3 and 10 abnormalities (p values<0.018, <0.013, <0.033, <0.026, <0.018, and <0.008, respectively) were present in the cancer patients than in the control subjects (See Table 3, hereinbelow and FIGS. 1A-F, 2A-D, 3A-E and 4A-D).

TABLE 3
Univariate logistic regression models for the outcome variable

| Variable | Coefficient | P-value | Odds Ratio |
|---|---|---|---|
| Age | 0.05 | 0.047 | 1.05(1.00-1.10) |
| Sex (male vs. female) | −0.34 | 0.494 | 0.71(0.27-1.89) |
| Smoking History (Pack Year) | | | |
| 0-<20 | — | — | |
| 20-50 | −0.9807 | 0.1022 | 0.38(0.12-1.22) |
| >50 | −0.4054 | 0.5687 | 0.67(0.17-2.69) |
| Deletions 3p22.1 | 0.17 | 0.081 | 1.19(0.98-1.43) |
| Abnormalities 3p22.1 | −40.03 | 0.865 | 0 |
| Monosomy cep3 | 0.16 | 0.459 | 1.18(0.77-1.80) |
| Polysomy cep 3 | 0.33 | 0.377 | 1.39(0.67-2.86) |
| Polysomy 3p22.1 | 0.72 | 0.146 | 2.06(0.78-5.8) |
| Deletions 10q22-23 | 0.44 | 0.014 | 1.55(1.09-2.19) |
| Abnormalities 10q22-23 | −0.14 | 0.806 | 0.87(0.27-2.73) |
| Monosomy cep10 | 0.15 | 0.277 | 1.16(0.89-1.51) |
| Polysomy cep10 | −0.41 | 0.444 | 0.67(0.24-1.89) |
| Polysomy 10q22-23 | 0.03 | 0.944 | 1.03(0.44-2.40) |
| All Abn 3 | 0.17 | 0.052 | 1.18(1.00-1.40) |
| Del and Poly 3p22.1 | 0.19 | 0.049 | 1.21(1.00-1.47) |
| Aneusomy 3 | 0.22 | 0.268 | 1.25(0.84-1.85) |
| All Abn 10 | 0.18 | 0.045 | 1.20(1.00-1.43) |
| Del and Poly 10q22-23 | 0.35 | 0.02 | 1.43(1.06-1.92) |
| Aneusomy 10 | 0.11 | 0.388 | 1.11(0.87-1.42) |
| All 3 and 10 abn | 0.15 | 0.01 | 1.17(1.04-1.31) |
| Cytological diagnosis | | | |
| Negative | | | |
| Squamous Metaplasia | 2.47 | 0.005 | 11.87(2.11-66.86) |
| Mild Dysplasia | 0.87 | 0.226 | 2.38(0.59-9.64) |
| Moderate Dysplasia | 2.12 | 0.019 | 8.31(1.41-49.06) |
| Severe Dysplasia | 1.56 | 0.106 | 4.75(0.72-31.37) |

Table 3: For abbreviations of 3, 3p22.1, 10q22-23 and 10, please see text under FISH section. Abn 3 = abnormalities chromosome 3; Abn 10 = abnormalities chromosome 10; Del = deletion; Poly = polysomy; The Odds Ratio (OR) are provided with the confident interval (CI) at 95%.

In a univariate logistic regression model estimating cancer status (Table 3, hereinabove), the most significant parameters were age, deletions of 3p and 10q, and a variety of abnormalities for 3p22.1 and 10q22-23 and chromosomes 3 and 10 as well as both squamous metaplasia and moderate dysplasia versus negative cytologic results.

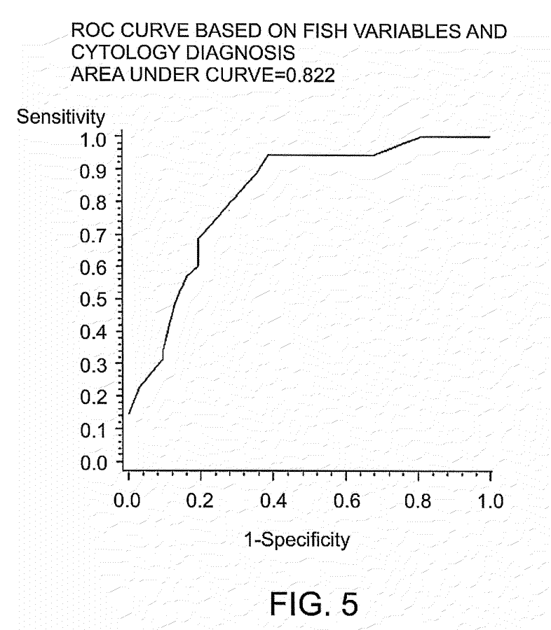

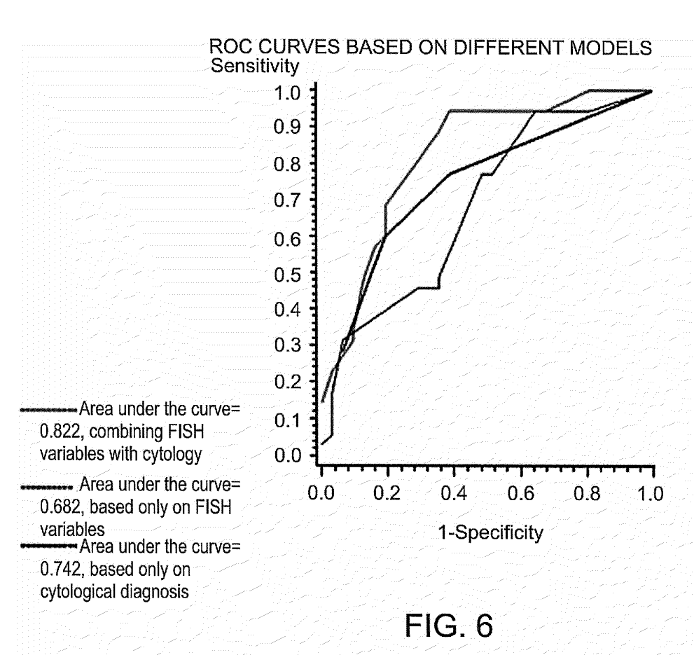

The multivariate logistic regression model to estimate cancer status (Table 4, hereinbelow) selected six variables, two genetic and four cytologic, as the most predictive parameters for estimating cancer status where the p value cutoff point was chosen as 0.10. The variable with the highest odds ratio (OR) was moderate dysplasia (OR 17.96) followed by squamous metaplasia (OR 14.84), severe dysplasia (OR 5.39), mild dysplasia (OR 3.63), deletion and polysomy of 10q22-23 greater or less than 2 (OR 4.38), and deletions and polysomies of 3p and centromeric 3 greater or less than 5 (OR 3.01). The ROC curve (FIG. 5), using a cutoff point of estimated p=0.004, showed the area under the curve to be 0.822 when using both the cytologic and FISH parameters. Using only the selected FISH variables of deletions of 10q and 3 and 3p abnormalities resulted in an ROC curve of 0.682 (p=0.065), whereas using only the cytologic diagnosis resulted in an ROC curve of 0.742 (p=0.040) (FIG. 6).

TABLE 4
Multivariate Logistic Regression Model

| | | | Odds Ratio |
|---|---|---|---|
| Variable | Coefficient | P-value | (95% CI) |
| Intercept | −2.677 | 0.002 | |
| All abnormalities 3/3p | 1.103 | 0.083 | 3.01(0.87-10.50) |
| Deletions and polysomies | 1.477 | 0.033 | 4.38(1.13-17.06) |
| 10/10q | | | |
| Cytological Diagnosis+ | 2.697 | 0.005 | 14.84(2.23-98.53) |
| (2 vs. 1) | | | |
| Cytological Diagnosis | 1.289 | 0.117 | 3.63(0.72-18.20) |
| (3 vs. 1) | | | |
| Cytological Diagnosis | 2.888 | 0.005 | 17.96(2.36-136.92) |
| (4 vs. 1) | | | |
| Cytological Diagnosis | 1.685 | 0.118 | 5.40(0.65-44.68) |
| (5 vs. 1) | | | |

Table 4: +Cytological Diagnosis: 1 = negative; 2 = squamous metaplasia; 3 = mild dyplasia; 4 = moderate dysplasia; 5 = severe dysplasia; vs. = versus.

If just moderate and severe dysplasia were considered to be predictors of the presence of lung cancer, then the sensitivity and specificity of the sputum cytology test were 32% and 87%, respectively. If only severe dysplasia was considered, the sensitivity of sputum cytology was 11% and the specificity, 99%.

Table 5 shows the actual probability for the presence of cancer in the sputum for each study participant using a combination of genetic variables and cytologic diagnosis performed on parallel microscopic slides (i.e., not on the same single cells). Assuming a cutoff of p>0.60 (p=a probability index) to indicate high risk for cancer, then 6/31 of the high-risk control subjects and 21/35 of the cancer patients appear to be at high risk for developing cancer,

TABLE 5
Estimates of probability of cancer for each patient by
sputum evaluation of genetic and cytologic variables

| | All | Deletions and | | | |
|---|---|---|---|---|---|
| | abnormalities | polysomies | Cytologic** | | Probability of |
| Patient* | 3/3p# | 10/10q## | Diagnosis | Cancer Status | cancer (p) |
| 1 | 1 | 0 | 3 | absent | 0.429 |
| 2 | 0 | 0 | 5 | absent | 0.271 |
| 3 | 0 | 1 | 1 | absent | 0.232 |
| 4 | 0 | 0 | 1 | absent | 0.064 |
| 5 | 0 | 1 | 1 | absent | 0.232 |
| 6 | 1 | 1 | 1 | absent | 0.476 |
| 7 | 1 | 1 | 3 | absent | 0.767 [symbol] |
| 8 | 1 | 1 | 1 | absent | 0.476 |
| 9 | 1 | 0 | 1 | absent | 0.172 |
| 10 | 0 | 0 | 3 | absent | 0.200 |
| 11 | 1 | 1 | 1 | absent | 0.476 |
| 12 | 1 | 1 | 5 | absent | 0.830 [symbol] |
| 13 | 0 | 1 | 1 | absent | 0.232 |
| 14 | 1 | 0 | 4 | absent | 0.788 [symbol] |
| 15 | 1 | 0 | 3 | absent | 0.429 |
| 16 | 0 | 1 | 2 | absent | 0.817 [symbol] |
| 17 | 0 | 0 | 1 | absent | 0.064 |
| 18C | 0 | 1 | 1 | absent | 0.232 |
| 19C | 0 | 0 | 1 | absent | 0.064 |
| 20C | 1 | 0 | 1 | absent | 0.172 |
| 21C | 0 | 0 | 1 | absent | 0.064 |
| 22C | 0 | 0 | 1 | absent | 0.064 |
| 23C | 0 | 1 | 1 | absent | 0.232 |
| 24 | 1 | 1 | 1 | absent | 0.476 |
| 25 | 0 | 0 | 1 | absent | 0.064 |
| 26 | 1 | 1 | 1 | absent | 0.476 |
| 27 | 0 | 1 | 4 | absent | 0.844 [symbol] |
| 28 | 0 | 1 | 1 | absent | 0.232 |
| 29 | 1 | 0 | 3 | absent | 0.429 |
| 30 | 0 | 0 | 3 | absent | 0.200 |
| 31 | 0 | 1 | 2 | absent | 0.817 [symbol] |
| 32 | 1 | 0 | 2 | present | 0.755 [symbol] |
| 33 | 1 | 1 | 2 | present | 0.931 [symbol] |
| 34 | 1 | 1 | 2 | present | 0.931 [symbol] |
| 35 | 1 | 1 | 2 | present | 0.931 [symbol] |
| 36 | 1 | 1 | 1 | present | 0.476 |
| 37 | 1 | 1 | 1 | present | 0.476 |
| 38 | 1 | 1 | 1 | present | 0.476 |
| 39 | 0 | 1 | 4 | present | 0.844 [symbol] |
| 40 | 1 | 0 | 1 | present | 0.172 |
| 41 | 0 | 1 | 3 | present | 0.522 |
| 42 | 1 | 1 | 3 | present | 0.767 [symbol] |
| 43 | 1 | 1 | 3 | present | 0.767 [symbol] |
| 44 | 1 | 1 | 5 | present | 0.830 [symbol] |
| 45 | 1 | 1 | 3 | present | 0.767 [symbol] |
| 46 | 1 | 0 | 4 | present | 0.788 [symbol] |
| 47 | 0 | 1 | 4 | present | 0.844 [symbol] |
| 48 | 1 | 1 | 1 | present | 0.476 |
| 49 | 0 | 1 | 3 | present | 0.522 |
| 50 | 0 | 0 | 2 | present | 0.505 |
| 51 | 0 | 1 | 5 | present | 0.619 [symbol] |
| 52 | 1 | 1 | 5 | present | 0.830 [symbol] |
| 53 | 0 | 1 | 2 | present | 0.817 [symbol] |
| 54 | 0 | 0 | 4 | present | 0.553 |
| 55 | 0 | 1 | 5 | present | 0.619 [symbol] |
| 56 | 0 | 1 | 2 | present | 0.817 [symbol] |
| 57 | 1 | 1 | 1 | present | 0.476 |
| 58 | 1 | 1 | 2 | present | 0.931 [symbol] |
| 59 | 0 | 1 | 2 | present | 0.817 [symbol] |
| 60 | 0 | 1 | 4 | present | 0.844 [symbol] |
| 61 | 1 | 1 | 1 | present | 0.476 |
| 62 | 1 | 1 | 1 | present | 0.476 |
| 63 | 1 | 1 | 4 | present | 0.942 [symbol] |
| 64 | 0 | 0 | 4 | present | 0.553 |
| 65 | 1 | 1 | 2 | present | 0.931 [symbol] |
| 66 | 0 | 0 | 3 | present | 0.200 |

Table 5: Subjects 1-31 are controls without cancer: subjects 1-17 and 24-31 are high risk controls, and subjects 18-23 are healthy, non-smoking controls (marked with “C”). Patients 32-66 have cancer;
#All abnormalities of centromeric 3/3p22.1 - if the number of all abnormalities of centromeric 3/3p22.1 is <5 = 0, if >5 = 1;
##All deletions and polysomies centromeric 10/10q22-23, if the number of all deletions and polysomies centromeric 10/10q22-23 <2 = 0, if >2 = 1;
**Cytologic Diagnosis: 1 = negative, 2 = squamous metaplasia, 3 = mild dysplasia, 4 = moderate dysplasia, and 5 = severe dysplasia.
Boxes marked with [symbol] refer to high risk controls (6/25) and patients with lung cancer (21/35) who have probability index score above the cutoff of 0.600 (having increased risk of lung cancer).

Comparison between genetic changes in sputum and paired bronchial brush, tumor, normal lung, and bronchial cells adjacent to tumor—Genetic changes in the TBB, NBB, TPP, NTP and TAB were correlated with those detected in the sputum samples from the 16 lung cancer patients who underwent resection. There were significant correlations between abnormalities of chromosome 10 and 10q22-23 in the sputum and those in the TBB (Table 6), while abnormalities for 3 and 3p correlated negatively with 10q deletions in the TAB, and NBB.

TABLE 6
Correlation between genetic abnormalities in sputum and at different sites in the
respiratory tract in 16 patients*

| | | | TBB del 10q22-23 |
|---|---|---|---|
| | TAB del 10q22-23 | NBB del 10q22-23 | Correlation |
| | Correlation Coefficient | Correlation Coefficient | Coefficient |
| Sputum | (P-value) | (P-value) | (P-value) |
| Polysomy cep3 | −0.728 (0.026) | 0.114 (0.673) | 0.189 (0.331) |
| Polysomy 3p22.1 | 0.370 (0.327) | −0.632 (0.0086) | −0.260 (0.331) |
| Del 10q22-23 | −0.100 (0.806) | 0.139 (0.609) | 0.521 (0.0385) |
| Deletion/polysomy | | 0.075 (0.784) | 0.532 (0.034) |
| Cen10 or 10q22-23 | | | |

Table 6:
TBB: bronchial brush on tumor side;
NBB: bronchial brush on non-tumor side;
TAB: touch preparations from bronchus adjacent to tumor;
*The Spearman association among FISH parameters; del: deletion

Summary of results—Automated scoring of genetic abnormalities for 3p22.1 and 10q22-23 by fluorescence in situ hybridization (FISH) and conventional cytology performed on parallel slides was done on sputa from 35 subjects with lung cancer, 25 high-risk smokers, and six healthy control subjects. Correlation of FISH abnormalities between sputum and bronchial epithelial cells from the main stem and adjacent to tumor bronchi was performed in 16 patients who underwent resection for lung cancer. A multivariate analysis selected variables that most accurately predicted lung cancer. A model of probability for the presence of lung cancer was derived for each subject.

Cells exfoliated from patients with lung cancer contained genetic aberrations and cytologic atypias at significantly higher levels than in those from control subjects. If just moderate and severe dysplasia were considered as predictors of the presence of lung cancer, then the sensitivity and specificity of the sputum cytology test was 31.4% and 87%, respectively. If only severe dysplasia was considered as predictor for lung cancer the sensitivity of cytology was 11.4% and the specificity 93.5% to predict the presence of cancer. Molecular abnormalities in sputum correlated significantly with those in bronchial cells from other sites within the respiratory tract, confirming the field effect. When combined with cytologic atypia (on parallel slides), a model of risk for lung cancer was derived that had 60% (21/35) sensitivity and 81% (25/31) specificity to predict the presence of lung cancer.

Conclusions: For diagnosing lung cancer in sputum, a combination of molecular and cytologic variables (performed independently on the same sputum samples but on parallel slides) was superior to using conventional cytology alone.

Analysis and discussion—Because of CT's high sensitivity but lack of specificity, it would be desirable to develop a minimally invasive test for genetic susceptibility that may assist in identifying those individuals at highest risk for developing lung cancer. Detecting obvious cytologic atypia in the sputum may be more reflective of neoplastic events in the central than the peripheral airways and, from this aspect, would be superior to CT, which does not easily detect central airway lesions. However, conventional cytologic sputum screening lacks sensitivity for various reasons, including difficulties to detect small atypical squamous cells, the fact that abnormal cells may not be shed from peripherally based lesions, the patient's inability to produce an adequate cough specimen, and contamination of the specimen by oral superficial squamous cells and bacteria.

In this study, the present inventors found that epithelial cells in spontaneously produced sputum from patients with lung cancer had significantly higher levels of chromosomal abnormalities in centromeric 3, 3p22.1, centromeric 10, and 10q22-23 than did sputum from an age-matched cohort of high-risk smokers who were clinically negative for lung cancer. Genetic abnormalities occurred in the epithelial cells of subjects with negative cytologic findings as well as in those from subjects with squamous metaplasia or mild, moderate, or severe dysplasia. Abnormalities of 10q22-23, centromeric 3, and 3p22.1 were selected as being significant predictors of lung cancer. Additionally, the presence of squamous metaplasia or mild, moderate, or severe dysplasia was shown to have a high OR of predicting for lung cancer. In current clinical practice, a cytologic diagnosis of squamous metaplasia or mild dysplasia would not be considered diagnostic for malignancy. The findings in this study demonstrate that squamous metaplasia and all degrees of dysplasia are present at a significant level in patients with lung cancer. Previous studies showed that cytology, with the exception of moderate and severe dysplasia, has low sensitivity in the detection of cancer.

Based on the present study, subjects in a high-risk group, with high probability scores derived from cytologic and FISH analyses according to the study's model, should undergo CT scanning. If the CT findings are negative, these subjects would be ideal candidates to undergo fluorescence bronchoscopy to exclude the presence of central airway preinvasive malignant lesions. In the present study there were 6 high risk patients without CT evidence of lung cancer that might qualify for bronchoscopy based on their probability scores. The finding of a low probability score in several of the lung cancer patients may reflect an inadequate sputum sample. Use of induced sputa and stricter criteria for adequacy, such as the presence of bronchial epithelial cells and a greater number of histiocytes (lung macrophages) may improve the accuracy of the test.

The results of this study validated the approach to measuring and quantitating molecular abnormalities in consecutive fields of epithelial cells that were not necessarily cytologically abnormal. The significant correlation between chromosomal abnormalities in epithelial cells exfoliated in sputum and in those obtained by bronchial brushing of the main stem bronchi confirmed the hypothesis that cellular genetic abnormalities of 3p22.1 and 10q22-23 reflect a field cancerization effect within the bronchial cells of individuals at high risk for developing cancer. This field effect is the result of susceptibility to genetic damage at 3p22.1 and 10q22-23, caused by carcinogens such as cigarette smoke or atmospheric pollutants, that persists most likely because of an impaired ability to repair the DNA damage. The present inventors have previously demonstrated that this field effect is more pronounced on the side of the tumor than on the contralateral side.

Other investigators tested epithelial cells in sputum by FISH using a commercial probe set for four different chromosomal regions (5p15, 6 μl-q11, 7p12 (including epidermal growth factor receptor), and 8q24 (including C-myc) and required a positive sputum diagnosis to be based on DNA copy number gains for at least two probes in a minimum of two or three cells. Using this approach, the sensitivity (50%) and specificity (81%) of FISH did not exceed the sensitivity of sputum cytology to detect lung cancer. Furthermore, with this probe set, heavy tobacco smokers and asbestos-exposed workers had FISH results similar to those seen in never-smokers, suggesting that the composition of these probes was not optimal for detecting early lung cancer in high-risk populations. In contrast, others reported that FISH combined with cytology led to an improved diagnosis of malignancy. In a recent study, combined genetic aberrations for genes HYAL2 and FHIT on chromosome 3p were found by FISH in 76% of sputa from patients with cancer but in only 47% of cases that were considered positive on cytology, demonstrating, like in the present study, that with an appropriate choice of probes, FISH can detect abnormal cells that may be undetectable by cytology.

The sputum probe set of 3p22.1 and 10q22-23 was selected on the basis of results of high-resolution comparative genomic hybridization analysis of cDNA microarrays in adenocarcinomas and squamous cell carcinomas that showed high levels of these deletions, relative to those in normal human bronchial epithelial cells, in almost all tumors tested. These same probes were subsequently tested by FISH in adenocarcinomas and squamous cell carcinomas and found to correlate significantly with the results of the comparative genomic hybridization.

Deletion of SP-A are frequent in lung cancer as well as in adjacent bronchi, normal lung, and bronchial cells from main stem bronchi on the normal and tumor sides. The present inventors have shown that deletions of SP-A in lung cancer cells are inversely related to telomere length and significantly associated with overexpression of the gene for hTERT and high telomerase expression. Furthermore, deletions of SP-A in bronchial cells adjacent to the tumor are significantly associated with a poor prognosis in early-stage lung cancer. SP-A is present in type 2 alveolar epithelial cells, which are considered lung cancer stem cells and are involved in alveolar repair after lung injury. Studies in a rat hyperoxia model identified a subpopulation of type 2 alveolar cells with high telomerase activity that were resistant to injury and capable of proliferation.

Chromosome 3p deletion is currently the most common finding in lung cancer, and it occurs more frequently in the lung tumor tissues of patients who smoke than it does in those of nonsmoking patients. Furthermore, allelic losses at one or more chromosome 3p21.3 locus are the most frequent chromosomal abnormalities detected in the bronchial epithelia of smokers and are detected even in normal bronchial mucosae of smokers. Therefore, deletions in this region have been proposed as useful markers in smoking-related target epithelia for assessing risk.

The use of FISH for diagnostic purposes has increased considerably in the last few years, primarily because FISH permits visualization and examination of genetic aberrations as rare events in a large number of cells that may have normal genetic composition. FISH is ideally suited for cytologic specimens such as sputum, which may be obtained spontaneously by coughing or induced by inhalation of nebulized saline. The major value of sputum biomarkers is to identify patients at high risk for cancer-related events, such as the development of premalignant lesions or early cancers, so that these patients may be subject to intense surveillance either by fluorescent bronchoscopic examination with removal of neoplastic lesions or by regular helical CT scanning of lungs to detect peripheral carcinomas. Additionally, this is an ideal population to benefit from the use of chemopreventive agents and smoking-cessation counseling.

In summary, the present inventors used an automated quantitative system to score FISH abnormalities in epithelial cells from non-induced sputum specimens from lung cancer patients, which resulted in a gallery of cells that could then be interactively classified in conjunction with morphologic findings. Software programs specific to the sputum application and the size of the probes and specific filter sets were used to maximize the accuracy of the testing. Correlating the sputum findings with disease state per individual, the present inventors discovered that epithelial cells in sputum from patients with NSCLC were cytologically and genetically abnormal relative to those from a high-risk control group with no CT evidence of lung cancer as well as healthy controls. On the basis of the results of the model for risk of lung cancer, which should be validated in a larger study, it may be concluded that the best predictive sputum assay for lung cancer will be a combination of morphologic characteristics determined cytologically and quantitation of molecular abnormalities in both atypical cells and morphologically normal cells.

### Example 2

### Classification of Cells which are Relevant for Fish Analysis In a Sputum Sample

Morphological stains which enable visualization of the morphological characteristics of the cells are used for classifying the cells present in the sputum sample to cells which are relevant for FISH analysis, i.e., the target cells for analysis by cytogenetical staining and non-relevant cells which are excluded from FISH analysis. A sputum sample may include cells of the lower airway tract (bronchial cells and cells lining the conductive passages of the lower respiratory tract), lung cells, cells of the upper respiratory tract (cells lining the upper part of the trachea, pharynx, larynx and mouth), blood cells and other cells. It should be noted that a sputum sample may also include abnormal cells, such as cells derived from the lower respiratory tract, bronchioles and lungs which underwent morphological changes (including reversible and non-reversible change). Non-limiting examples of such cells are squamous metaplasia cells, squamous atypia and squamous dysplasia cells.

I. Cells which are Relevant for Analysis (Targeted for a Subsequent FISH Analysis)

Respiratory cells—Cells of the conductive passages of the lower respiratory tract which can be found in a sputum sample include, but are not limited to, respiratory epithelial cells such as goblet cells, ciliated cells and non-ciliated cells.

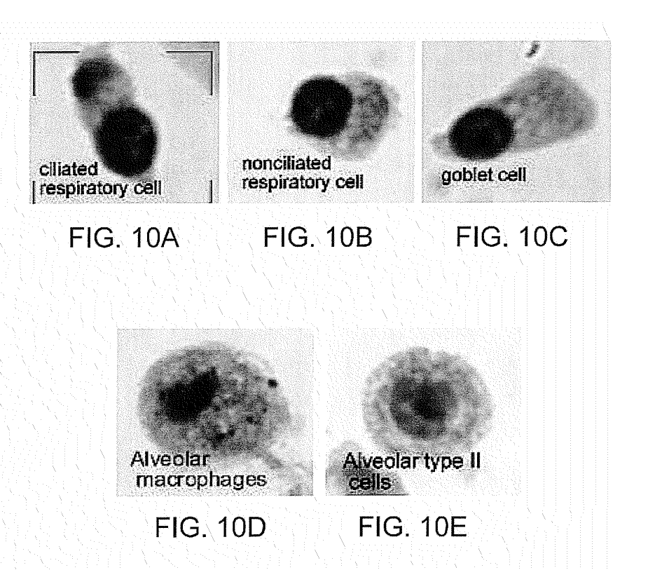

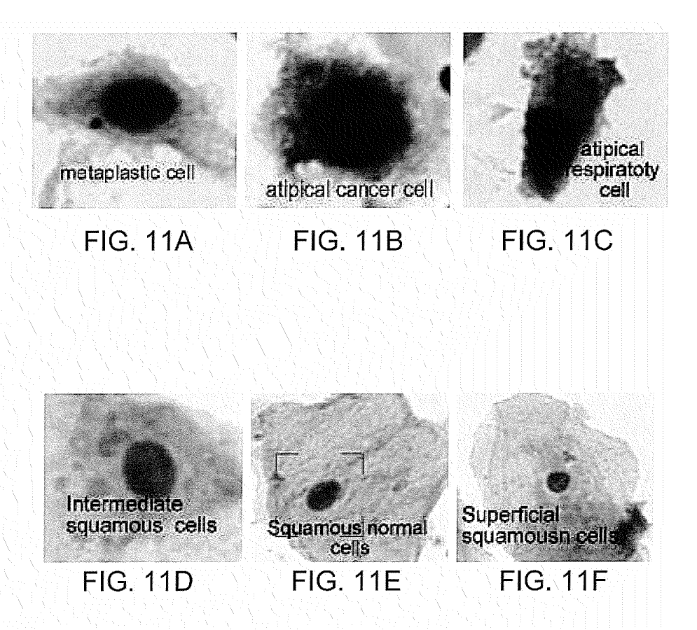

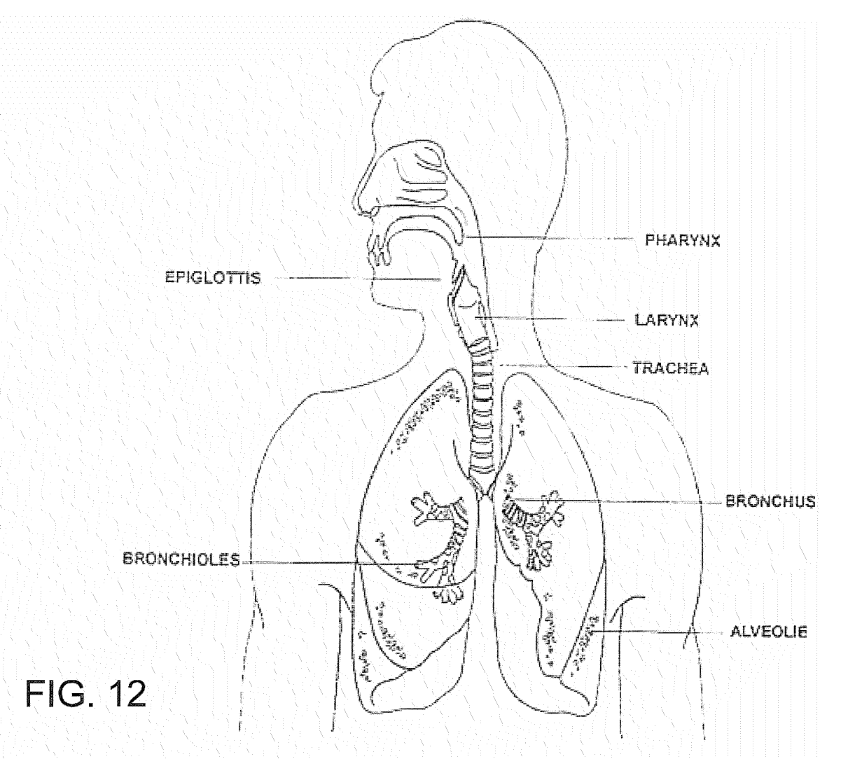

Goblet cells—are columnar cells with vacuolated, faintly stained basophilic cytoplasm and peripheral nucleus (see for example, FIG. 10C).

Ciliated cells—have a length which is at least twice of their width, having variable size are recognized by their terminal plate, cilia and tapering ends (see for example, FIG. 10A).

Non-ciliated cells (also called Clara cells)—are serous glandular cells that secrete a surfactant-like material that appears to coat and protect the bronchiolar lining (see for example, FIG. 10B). The non-ciliated cells are bronchial epithelial cells that don't have cilia.

When reaching the smallest bronchioles, goblet cells disappear while there are still ciliated cells present.

Cells of the lung which may be included in a sputum sample include epithelial cells of the alveoli (Alveolar type I cells and Alveolar type II cells) and alveolar macrophages.

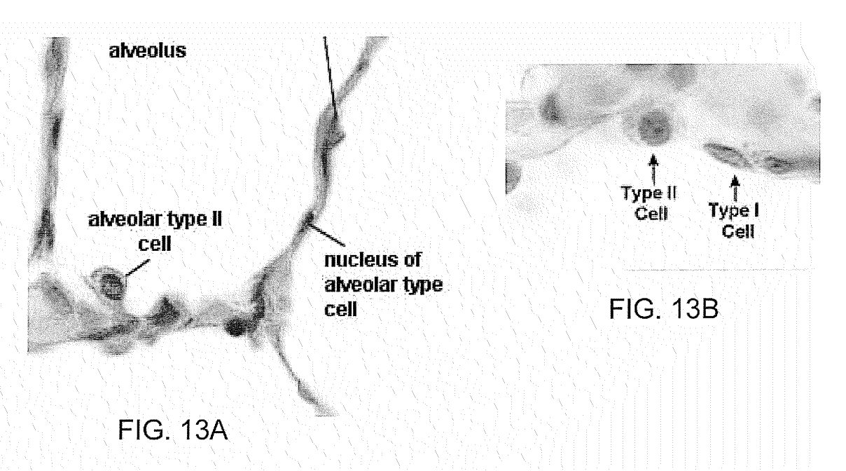

Alveolar type I cells—Small alveolar cells or type I pneumocytes are extremely flattened (the cell may be as thin as 0.05 μm) and form most (95%) of the surface of the alveolar walls (see for example, FIG. 13B).

Alveolar type II cells—large alveolar cells or type II pneumocytes, are small, round, single cells (appear alone, as compared with Alveolar type I cells which line side by side and form surface of the alveolar walls). The Alveolar type II form small bulges on the alveolar walls with vacuolated cytoplasm and central nuclei having one to two nucleoli, contain large number of granules called cytosomes (or multilamellar bodies), which consist of precursors to pulmonary surfactant (see FIGS. 13A and 13B).

Alveolar macrophages—The non-epithelial cells are predominantly pulmonary macrophages derived from the alveoli. Invariably these contain variable amounts of black granular material or dust. Their presence indicates adequacy of sputum specimen.

II. Identification of respiratory tract cells of a sputum sample having abnormal morphology—In the presence of consistent irritation of the airway tract and the lungs such in the case of tobacco smoke or inhalation of other pollutants, a series of morphological changes occur that may lead to the progression of carcinoma. Early changes include a loss of the ciliated columnar epithelium, basal cell hyperplasia, and the formation of a low columnar epithelium without cilia. These changes are followed by a squamous metaplasia. Metaplasia is the reversible replacement of one differentiated cell type with another mature differentiated cell type, while Atypia and Dysplasia are a clinical term for irreversible abnormality in a cell that can develop to cancer. As cellular atypia develops and advances there is progression through mild, moderate and severe dysplasia to carcinoma. Therefore, a sputum sample may include various cell types derived from bronchial epithelium representing the cytology changes as cells progress along the multistep pathway from inflammation to lung cancer.

Squamous metaplasia cells—are bronchial epithelial cells in which the normal ciliated columnar shape is replaced by a squamous epithelium shape. This transformation from a glandular epithelium to squamous epithelium is known as squamous metaplasia.

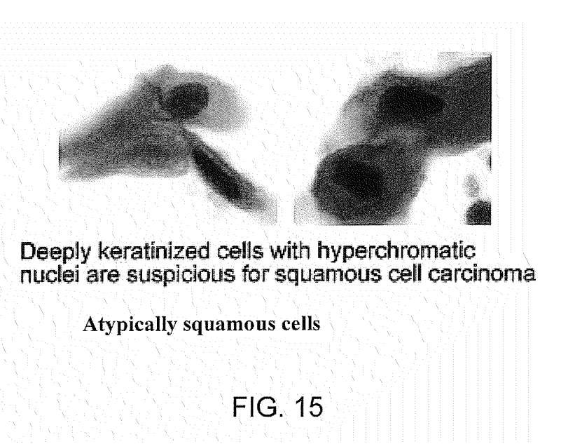

Squamous atypia cells (atypical squamous metaplastic)—Atypia is a clinical term for abnormality in a squamous cell. It may or may not be a precancerous indication associated with later malignancy, but the level of appropriate concern is highly dependent on the context with which it is diagnosed (See FIG. 15).

Squamous metaplastic cells—are considered suspicious for squamous cell carcinoma as their nuclei become hyperchromatic and angulated, (see e.g., FIGS. 7A-C).

Squamous dysplasia cells—Squamous dysplasia is the earliest form of pre-cancerous lesion recognizable, characterized by the presence of at least some squamous features in the cytoplasm of the abnormal cells. These include a sharp border, orange, or deep basophilic staining of the cytoplasmic keratin, and filaments of keratin ringing the outer diameter of the cell.

The grade of dysplasia mirrors the maturity of the cells involved. For example, cells of mild dysplasia resemble mature metaplastic, superficial and intermediate cells while more severely dysplastic epithelium reflects less mature normal epithelium such as parabasal and/or immature metaplastic type cells.

Mild dysplasia involves chiefly the deeper layers of the epithelium (inner one third of the epithelial thickness). In moderate dysplasia the abnormal changes move toward the surface involving the inner two thirds of the thickness of the epithelium.

Severe dysplasia is used by some as synonymous with squamous carcinoma in situ while others use the term to describe changes including almost all of the epithelium, but falling just short of carcinoma in situ.

Cellular changes in dysplasia are those of nuclear pleomorphism, hyperchromia (increase in nuclear chromatin) causing deeper nuclear staining, prominent nucleoli, increased nuclear-cytoplasmic ratio, increased mitoses, loss of cellular polarity and crowding of cells.

III. Cells of a Sputum Sample which are not Relevant for a Subsequent Cytogenetical (FISH) Analysis (Non-Targeted Cells, Excluded from Analysis)

Cells of the upper respiratory tract which may be included in the sputum sample include squamous epithelial cell.

Squamous epithelial normal cells—Squamous epithelial cell are irregularly shaped and very flat cells, such as superficial squamous epithelial cells and intermediate squamous cells (see e.g., FIG. 7J).

Blood cells—Red and white blood cells might be present in the sputum due to infection or irritation of the respiratory tract. These cells may be recognized by their small size compared to the other cell types found in sputum. Red blood cells are a-nucleated, and the white blood cells include lymphocytes, polymorphonuclear (PMN) cells, and other white blood cells (WBC) (se FIG. 14 for exemplary blood cells).

As described in the Background section multiple clonal abnormalities arising within lower airway tract and lung cells are associated with lung cancer the cells-of-interest which are identified in the sputum sample by the morphological stain include, but are not limited to lower airway tract cells such as goblet cells, ciliated cells and non-ciliated cells; and lung cells such as alveolar type I cells, alveolar type II cells and alveolar macrophages.

Cells of a sputum sample which are excluded from the subsequent FISH scan (non-targeted cells) include normal squamous epithelial cells and blood cells.

### Example 3

### Determination of Significant Parameters for Combined Analysis of Morphology and Fish for Detection of Lung Cancer in Induced Sputum

Evaluation of induced sputum samples from 33 subjects: 15 patients diagnosed with lung cancer and from 18 healthy non-smoking controls.

Materials and Methods

Study Design and Patient Population—The local ethical review committee approved the study and informed consent was obtained from all patients. All patients had bronchoscopy. Final diagnosis of cancer based on histology or cytology material from bronchoscopy, transthoracic fine needle aspiration or surgical specimen. Sputum samples were collected before bronchoscopy. In the cancer patients who underwent resection, sputum samples were collected before surgery. All participants underwent induced sputum production after saline inhalation with an ultrasonic nebulizer, to maximize the yield of cells from the airways. Sputum samples were analyzed blindly.

Sputum production and processing—Sputum samples were collected in a container filled with Sacommano's fixative (50% alcohol which contains 2% Carbowax). Sputum samples were washed in PBS×1, centrifuged and the cell pellet was resuspended in sputolysin for 15 minutes at 37° C. After another centrifugation, cell pellet was washed again in PBS×1, and placed onto silane-coated glass slides using cytocentrifuge (Shandon Cytospin 2, Thermo Fisher Scientific, Inc., Waltham, Mass.) in 50% ethanol (EtOH). Cytospin slides were fixed in 95% alcohol and were kept wrapped in aluminum fold at −20° C. until further processing. The sediment was used to prepare at least 12 cytospin slides.

Morphological staining—Slides were stained in May-Grünwald Giemsa stain (Sigma, St Louis, Mo., USA) and air-dried.

Destaining and pretreatment—Slides were immersed in Carnoy's fixative for 1 hour and washed once in 1×PBS, 5 minutes each. The, slides were digested in 10 mM HCl/0.05% digestion enzyme (BioView Ltd. Rehovot, Israel) for approximately 15 minutes at 37° C., following which the slides were washed in 1×PBS for 5 minutes, fixed in 1% formaldehyde/PBS for 5 minutes, washed twice in 1×PBS for 5 minutes each and dehydrated in an ice-cold ethanol series (70, 80, 100%).

FISH—Two bacterial artificial chromosome (bac) clones, commercially available from Invitrogene (Carlsbad, Calif.) were used for the FISH analysis:
1. RP11-506MI3—clone of 180 kb, located at 10q22.2-q23.1 and confirmed to contain genomic sequences of SP-A comprising both SFTPA1 and SFTPA2
2. RP11-391M1 clone located at 3p22.1, containing almost 200 kb of genomic sequences and consisting of four known genes—RPL 14, CD39A, GC20 translation factor sui1 homolog, and PMGM.

Clones were isolated and labeled with Spectrum Green dUTP (Vysis Inc., Downers Grove, Ill.) according to the manufacturer's instructions. Localization of the bacterial artificial chromosome clone on chromosomes 3 and 10 was confirmed by using normal metaphase FISH.

A two-color FISH assay using the Spectrum Green labeled bac probes, combined with commercial centromeric probes for chromosomes 3 and 10 labeled in Spectrum Orange (cep 3 and cep 10; Vysis, Inc., Downers Grove, Ill.) was performed on two separate slides. One hundred nanograms of each labeled probe was mixed with an equal quantity of human Cot-1 DNA (Life Technologies, Rockville, Md.) in 10 μl of LSI hybridization buffer (Vysis) and mounted on a slide together with 1 μl of either cep 3 (for 3p22.1) or cep 10 (for 10q22-23). The probe and target DNA were co-denatured at 74° C. for 4 minutes and hybridized at 37° C. overnight in a humidified chamber. Post-hybridization washes were performed in 0.4×SSC for 2 minutes at 75° C. followed by 2×SSC/0.1% NP-40 at room temperature. Slides were counterstained in BlueView (BioView Ltd, Rehovot, Israel) and evaluated under a fluorescence microscope equipped with the appropriate filter combinations. If the hybridization signals were deemed satisfactory, the slides were sent for automated FISH scanning. Counterstaining of nuclei was performed with DAPI and evaluated under a fluorescent microscope equipped with the appropriate filter combinations.

Automated FISH Scanning—Slides were automatically scanned by the Duet™ (BioView Ltd. Rehovot, Israel). The Duet™ is based on a fully automated microscope (Olympus BX61, Japan), a motorized 8-slides stage (Marzhauser, Wetzler, Germany) and a 3CCD progressive scan color camera (JVC KYF75U, Japan). The system allows the same slide to be scanned twice in two different staining: morphology and FISH. The coordinates and images of all cells found in the first scan are saved and matched to the fluorescent images of the second scan. Morphology scans were performed in bright-light using ×20 dry objective. FISH scanning was performed using ×60 oil objective in fluorescent illumination and by using appropriate filters and a software program specifically designed to capture and analyze the specific signal patterns generated by our probes. While scanning, the system produces images of all captured cells that can be further reviewed by the operator.

“Area scan”—Slides were scanned under fluorescent filters (Dark field imaging) suitable for viewing the FISH probes. All cells were classified according to a six tiered scoring system as follows: “Normal cells”—two copies of the gene and two centromeres, “deletion 1”—one copy of the gene is missing, “deletion 2”—two copies of the gene are missing, “Abnormal”—one extra copy of the centromere compared to the gene, “Polysomy, Extra Gene”—more than two copies of the gene and the centromeres.

“Target scan”—In order to correlate morphological changes with genetic aberrations on the same single cell the “target scan” was applied. Slides were first scanned under Bright field scan to identify relevant cells (i.e., cells exhibiting morphological characteristics typical to pre-defined cell types, e.g., as described in FIGS. 7A-I; and Example 2) which excluded squamous epithelial cells and blood cells and then the identified cells were confirmed manually and re-scanned by fluorescent mode to identify abnormalities by FISH staining. FISH abnormalities were classified to “Normal cells”; “deletion 1”; “deletion 2”; “Abnormal”; “Polysomy, Extra Gene” as described hereinabove.

Cells were classified according to the classes specified in the six tiered scoring system (Franklin W A et al. WHO classification of tumors of the lung, pleura, thymus and heart, IARC press: lyon, 2004, pp. 68-72).

Two slides were scanned for each sputum samples: one slide was hybridized to the 3p and its control centromere and the second slide was hybridized to the 10q probe and centromere 10. Each slide was scanned twice: 1) Area scan—at least 300 consecutive epithelial cells were selected randomly and analyzed, regardless of their morphology, and 2) Target scan—“target cells” were defined as normal and atypical cells derived from the airways. Following morphology scan, cells were subjected into the “target cells” class (both manually and automatically) based on their morphology. During FISH scan, only the pre-selected “target cells”, were automatically scanned and classified by their signal pattern. Samples with less than 50 target cells were excluded from the study.

For each cell, the presence or absence of centromeric probes (orange signals) or locus-specific probes (green signals) was recorded. Cells were classified into 3 major sub-groups: a) Normal—displaying 2 centromeric and 2 locus-specific signals; b) Deletion—displaying 2 centromeric and 1 locus specific signals; c) Polysomy—displaying multiple gains of both centromere and locus specific signals.

Only cells that were clearly non-overlapping and complete, with well-preserved nuclei, with high-quality fluorescence signals and without background fluorescence were scored. At the end of each scan, the images of all cells that were scanned and analyzed were displayed and a pie chart summarizing the chromosomal abnormalities found in each scan was generated. Two experienced observers, who were trained on the system and blinded to the patients' clinical status confirmed the automatic classification and reclassified cells that were misinterpreted by the system.

Inclusion and exclusion criteria—All patients included in the study had cytological or histological approved cancer. None of the participants had received prior diagnosis radiotherapy or chemotherapy. Following the morphology scan, the adequacy of the sample was evaluated by an expert technician. Slides were considered insufficient for diagnosis if they had less than 50 relevant target cells; including atypical cells, normal bronchial epithelial cells, and metaplastic cells. Slides with insufficient hybridization (i.e. weak signals, high fluorescent background, etc) were excluded from the study as well.

Statistical Analyses—All the parameters were tabulated using descriptive statistics. Simple comparison between the groups was done using the non-parametric Wilcoxon's rank-sum test. Sensitivity/specificity analysis was done by searching for the cutoff that yields the highest sensitivity given fixed specificity of at least 80%. This was performed once for the entire data and once for data excluding outlying observations. A value was considered to be an outlier if it was higher than 1.5 times the inter-quartile range above the 3rd quartile. This method was chosen as this study is an exploratory study aimed to detect any possible diagnostic parameters

Data Description—Data was recorded in Excel file which included patient identification number (ID), Diagnosis (Control/Lung Cancer) and 30 parameters, divided into Target scan parameters and Area scan parameters. Fifteen (15) subjects with diagnosed lung cancer and 18 healthy non-smoking controls were blindly tested.

Description of parameters—

3p # targets—Number of total relevant cells (target cells) scored using the 3p and 3 cen probe;

3p # del—Cells showing 2 centromeric signals of chromosome 3 (in red) and one green signal at 3p;

3p # Abn.—Cells showing 3 centromeric signals of chromosome 3 (in red) and 2 green signals at 3p (chromosomal gain and deletion of 3p locus);

3p # Poly—Cells showing >2 centromeric signals of chromosome 3 (in red) and >2 green signals of 3p locus;

Total 3p—Sum of genetic abnormality for chromosome 3 found (3p # del, 3p # Abn, 3p # Poly);

Total 3p (−Abn.)—Sum of 3p # del and 3p # Poly);

10q # targets—Number of total relevant cells (target cells) scored using the 10q and 10 cen probe;

10q # del—Cells showing 2 centromeric signals of chromosome 10 (in red) and one green signal at 10q;

10q # Abn.—Cells showing 3 centromeric signals of chromosome 10 (in red) and 2 green signals at 10q (chromosomal gain and deletion of 3p locus);

10q # Poly—Cells showing >2 centromeric signals of chromosome 10 (in red) and >2 green signals of 10q locus;

Total 10q—Sum of genetic abnormality for chromosome 10 found (10q # del, 10q # Abn, 10q # Poly);

Total 10q (−Abn.)—Sum of 10q # del and 10q # Poly);

Total 3p, 10q—Sum of genetic abnormality for chromosome 3 and 10 found (3p & 10q # del, 3p & 10q # Abn, 3p & 10q # Poly);

Total 3p, 10q (−Abn.)—Sum of 3p & 10q # del and 3p & 10q # Poly);

LAV # Abn.—Number of cells showing abnormality (polysomies) using the LAVysion prone kit;

Total 3p (−Abn.)/3p # targets—Sum of 3p # del and 3p # Poly) divided by the number of total relevant cells (target cells) scored using the 3p and 3 cen probe;

Total 3p/3p # targets—Sum of genetic abnormality for chromosome 3 found (3p # del, 3p # Abn, 3p # Poly) divided by the number of total relevant cells (target cells) scored using the 3p and 3 cen probe;

Total 10q (−Abn.)/10q # targets—Sum of 10q # del and 10q # Poly) divided by the number of total relevant cells (target cells) scored using the 10q and 10 cen probe;

Total 10q/10q # targets—Sum of genetic abnormality for chromosome 10 found (10q # del, 10q # Abn, 10q # Poly) divided by the number of total relevant cells (target cells) scored using the 10q and 10 cen probe;

Statistical Analysis Methods

Sensitivity/Specificity Analysis—All the parameters were tabulated using descriptive statistics. Simple comparison between the groups was done using the non-parametric Wilcoxon's rank-sum test. Four additional measures were calculated based on the Target statistics and were added to the descriptive analysis. Those were:
1. Total 3p/3p # targets, i.e., the total chromosome 3 (i.e., centromere 3+3p22.1) genetic aberrations identified in a sample out of the total cells in the sample targeted for analysis by FISH;
2. Total 3p (−abn.)/3p # targets, i.e., the total chromosome 3 genetic aberrations not including the class of 3p22.1 abnormalities (i.e., 3 signals of the centromeric probe of chromosome 3 and 2 signals of the 3p22.1, which reflects amplification of chromosome 3 with a deletion of the 3p22.1 locus) identified in a sample out of the total cells in the sample analyzed by FISH.
3. Total 10q/10q # targets i.e., the total chromosome 10 genetic aberrations (i.e., centromere 10 and 10q22-23) identified in a sample out of the total cells analyzed by FISH in the sample.
4. Total 10q (−Abn.)/10q # targets, i.e., the total chromosome 10 genetic aberrations not including the class of 10q22-23 abnormalities [i.e., 3 signals of the centromeric 10 probe and 2 signals of the 10q22-23, which reflects amplification of chromosome 10 with a deletion of the 10q22-23 locus) identified in a sample out of the total cells analyzed by FISH in the sample.

Sensitivity/specificity analysis was done first using a single parameter, simply by searching for the cutoff that yields the highest Youden's Index (defined as the sum of sensitivity and specificity) or the cutoff that yields the highest sensitivity given a fixed specificity of at least 80%. This was performed once for the entire data and once for data excluding outlying observations (outlying values). A value was considered to be an outlier if it was higher than 1.5 times the inter-quartile range above the 3^{rd} quartile. A list of all outliers is presented in Table 20.

The same sensitivity/specificity analysis was performed using a combination of two parameters as described below. The optimal cutoffs (one for each parameter) were the combination of two cutoffs that yielded the highest Youden's Index given two types of decision rules:

Decision rule 1: A subject is classified as having lung cancer if both parameters are above their respective cutoffs.

Decision rule 2: A subject is classified as having lung cancer if at least one of the two parameters is above its respective cutoff.

The comparison of the prediction power between the tested parameters and LAVysion probe kit (Vysis) was assessed by comparing the proportion of true predictions (number of true positive+number of true negative divided by the total number of subjects) using logistic regression.

Both the single parameter analysis and the two parameters analysis were repeated using a subset of the data that excludes subjects with less than 50 targets (i.e., less than 50 relevant cells for targeted scan) collected to calculate the target statistics. Six subjects were excluded from this analysis—3 from the Control group (IS-107, IS-110, IS-153) and 3 from the Lung Cancer group (IS-10, IS-21, IS-150).

Power Analysis—Power analysis was performed to evaluate the number of subjects required to achieve 80% power under different scenarios. Various “true” sensitivity rates were compared to a constant proportion using an exact binomial two-sided test.

Results

Descriptive statistics—The following Tables present descriptive statistics for each parameter along with the p-value from the Wilcoxon test. Table 7, presents the Target parameters (using target scan) and Table 8 presents the Area parameters.

TABLE 7
Descriptive statistics for target parameters by diagnosis

| Parameter/Diagnostic | | Mean | Std | Min | Median | Max | N | P-value |
|---|---|---|---|---|---|---|---|---|
| 3p # targets | Lung Cancer | 193.7 | 126.9 | 14.0 | 152.0 | 409.0 | 15 | 0.9856 |
| | Control | 237.8 | 218.8 | 39.0 | 150.5 | 732.0 | 18 | |
| 3p # del | Lung Cancer | 6.4 | 8.3 | 0.0 | 2.0 | 31.0 | 15 | 0.3300 |
| | Control | 2.7 | 2.7 | 0.0 | 2.0 | 10.0 | 18 | |
| 3p # Abn. | Lung Cancer | 9.0 | 15.3 | 0.0 | 4.0 | 60.0 | 15 | 0.5136 |
| | Control | 9.5 | 11.7 | 0.0 | 6.0 | 47.0 | 18 | |
| 3p # Poly | Lung Cancer | 11.9 | 13.6 | 0.0 | 6.0 | 45.0 | 15 | 0.0319** |
| | Control | 4.5 | 7.5 | 0.0 | 2.0 | 32.0 | 18 | |
| Total 3p | Lung Cancer | 27.3 | 24.8 | 0.0 | 16.0 | 95.0 | 15 | 0.1285 |
| | Control | 16.7 | 17.8 | 2.0 | 10.5 | 69.0 | 18 | |
| Total 3p (-Abn.) | Lung Cancer | 18.3 | 14.3 | 0.0 | 11.0 | 45.0 | 15 | 0.0049** |
| | Control | 7.2 | 8.6 | 1.0 | 4.0 | 35.0 | 18 | |
| 10q # targets | Lung Cancer | 163.7 | 130.7 | 20.0 | 141.0 | 527.0 | 15 | 0.8282 |
| | Control | 225.1 | 232.8 | 36.0 | 146.0 | 767.0 | 18 | |
| 10q # del | Lung Cancer | 2.4 | 3.7 | 0.0 | 1.0 | 12.0 | 15 | 0.7779 |
| | Control | 2.6 | 3.5 | 0.0 | 1.0 | 13.0 | 18 | |
| 10q # Abn. | Lung Cancer | 2.1 | 3.3 | 0.0 | 1.0 | 12.0 | 15 | 0.6573 |
| | Control | 1.4 | 2.6 | 0.0 | 0.5 | 11.0 | 18 | |
| 10q # Poly | Lung Cancer | 13.0 | 19.8 | 0.0 | 5.0 | 67.0 | 15 | 0.1108 |
| | Control | 2.7 | 2.5 | 0.0 | 2.0 | 8.0 | 18 | |
| Total 10q | Lung Cancer | 17.5 | 25.3 | 0.0 | 5.0 | 91.0 | 15 | 0.3935 |
| | Control | 6.7 | 6.3 | 0.0 | 5.0 | 20.0 | 18 | |
| Total 10q (-Abn.) | Lung Cancer | 15.4 | 22.6 | 0.0 | 5.0 | 79.0 | 15 | 0.4126 |
| | Control | 5.3 | 4.9 | 0.0 | 4.0 | 16.0 | 18 | |
| Total 3p, 10q | Lung Cancer | 44.8 | 41.4 | 0.0 | 33.0 | 135.0 | 15 | 0.1427 |
| | Control | 23.4 | 21.4 | 3.0 | 17.0 | 80.0 | 18 | |
| Total 3p, 10q (-Abn.) | Lung Cancer | 33.7 | 34.5 | 0.0 | 25.0 | 117.0 | 15 | 0.0286** |
| | Control | 12.4 | 12.5 | 2.0 | 8.5 | 51.0 | 18 | |
| LAV # Abn. | Lung Cancer | 13.1 | 17.7 | 0.0 | 8.0 | 62.0 | 15 | 0.0103** |
| | Control | 1.5 | 2.6 | 0.0 | 0.0 | 10.0 | 17 | |
| Total 3p (-Abn.)/3p # targets | Lung Cancer | 0.1 | 0.1 | 0.0 | 0.1 | 0.4 | 15 | 0.0006** |
| | Control | 0.0 | 0.0 | 0.0 | 0.0 | 0.1 | 18 | |
| Total 3p/3p # targets | Lung Cancer | 0.2 | 0.1 | 0.0 | 0.1 | 0.5 | 15 | 0.0315** |
| | Control | 0.1 | 0.0 | 0.0 | 0.1 | 0.2 | 18 | |
| Total 10q (-Abn.)/10q # targets | Lung Cancer | 0.1 | 0.1 | 0.0 | 0.0 | 0.6 | 15 | 0.2540 |
| | Control | 0.0 | 0.0 | 0.0 | 0.0 | 0.1 | 18 | |
| Total 10q/10q # targets | Lung Cancer | 0.1 | 0.1 | 0.0 | 0.1 | 0.6 | 15 | 0.2540 |
| | Control | 0.0 | 0.0 | 0.0 | 0.0 | 0.1 | 18 | |

Table 7: Presented are the mean number of cells with genetic aberrations analyzed by the target scan as well as the mean ratio of the cells with genetic aberrations out of the total analyzed cells by the target scan, along with the standard deviation (std), minimum value (Min), median value or Maximum value (Max) for each group (lung cancer or control).
N = the number of subjects in each group.
**= indicates p value smaller than 0.05 (a significant difference between the groups);
*indicates p values between 0.05 and 0.10 (a trend towards significance).

TABLE 8
Descriptive statistics for area parameters by diagnosis

| Parameter/Diagnostic | | Mean | Std | Min | Median | Max | N | P-value |
|---|---|---|---|---|---|---|---|---|
| 3p # del | Lung Cancer | 1.9% | 1.8% | 0.0% | 1.6% | 5.9% | 14 | 0.4700 |
| | Control | 1.3% | 1.2% | 0.0% | 1.0% | 5.0% | 18 | |
| 3p # Abn. | Lung Cancer | 2.1% | 2.3% | 0.0% | 1.6% | 7.7% | 14 | 0.7040 |
| | Control | 1.7% | 1.7% | 0.0% | 0.9% | 6.5% | 18 | |
| 3p # Poly | Lung Cancer | 1.4% | 1.8% | 0.0% | 0.9% | 7.3% | 14 | 0.0771* |
| | Control | 0.8% | 1.2% | 0.0% | 0.4% | 5.3% | 18 | |
| Total 3p | Lung Cancer | 5.5% | 3.3% | 2.0% | 4.8% | 14.8% | 14 | 0.0461** |
| | Control | 3.9% | 3.1% | 1.0% | 2.9% | 12.0% | 18 | |
| Total 3p (-Abn.) | Lung Cancer | 3.4% | 2.5% | 0.0% | 3.1% | 8.6% | 14 | 0.1436 |
| | Control | 2.1% | 1.5% | 0.2% | 1.9% | 5.7% | 18 | |
| 10q # del | Lung Cancer | 1.8% | 1.3% | 0.0% | 1.5% | 4.0% | 14 | 0.0574* |
| | Control | 1.0% | 0.9% | 0.0% | 0.8% | 3.2% | 18 | |
| 10q # Abn. | Lung Cancer | 1.0% | 1.0% | 0.1% | 0.8% | 3.3% | 14 | 0.0043** |
| | Control | 0.4% | 0.7% | 0.0% | 0.2% | 2.8% | 18 | |
| 10q # Poly | Lung Cancer | 1.7% | 1.9% | 0.0% | 1.0% | 6.7% | 14 | 0.0099** |
| | Control | 0.3% | 0.4% | 0.0% | 0.2% | 1.2% | 18 | |
| Total 10q | Lung Cancer | 4.5% | 2.7% | 1.1% | 4.3% | 9.3% | 14 | 0.0013** |
| | Control | 1.7% | 1.7% | 0.0% | 1.1% | 6.8% | 18 | |
| Total 10q (-Abn.) | Lung Cancer | 3.5% | 2.2% | 0.9% | 3.6% | 8.9% | 14 | 0.0009** |
| | Control | 1.3% | 1.1% | 0.0% | 0.9% | 4.0% | 18 | |
| Total 3p, 10q | Lung Cancer | 10.0% | 4.7% | 4.2% | 9.0% | 18.6% | 14 | 0.0066** |
| | Control | 5.6% | 4.1% | 1.0% | 5.1% | 16.5% | 18 | |
| Total 3p, 10q (-Abn.) | Lung Cancer | 6.9% | 3.8% | 2.3% | 5.4% | 12.4% | 14 | 0.0041** |
| | Control | 3.5% | 2.3% | 0.8% | 2.8% | 8.9% | 18 | |
| LAV # Abn. | Lung Cancer | 10.6% | 15.7% | 1.0% | 6.0% | 62.0% | 14 | 0.0159** |
| | Control | 2.8% | 3.7% | 0.0% | 2.0% | 14.0% | 16 | |

Table 8: Presented are the mean ratio of the cells with genetic aberrations out of the total analyzed cells by the area scan along with the standard deviation (std), minimum value (Min), median value or Maximum value (Max) for each group (lung cancer or control).
N = the number of subjects in each group.
**= indicates p value smaller than 0.05 (a significant difference between the groups);
*indicates p values between 0.05 and 0.10 (a trend towards significance).

Sensitivity/Specificity Analysis

Single parameter—The following Tables 9-10 present results of sensitivity and specificity based on a single parameter. For each parameter the cutoff that yields the maximal Youden's Index (=sensitivity+specificity) is presented. For all parameters, the decision rule is to classify a subject as having lung cancer if his or her measured value is above the cutoff. Results are presented for all data and for data excluding outliers. Table 9 presents results for Target parameters (using target scan) and Table 10 for Area parameters (using area scan).

TABLE 9
Sensitivity and specificity results for target parameters (single parameter)

| | All Data | | | | Without Outliers | | | |
|---|---|---|---|---|---|---|---|---|
| | | | | Youden's | | | | Youden's |
| Parameter | Cutoff | Sensitivity | Specificity | Index | Cutoff | Sensitivity | Specificity | Index |
| 10q # Abn. | 2 | 33.3% | 83.3% | 116.7% | 2 | 23.1% | 88.2% | 111.3% |
| 10q # Poly | 6 | 40.0% | 94.4% | 134.4% | 6 | 18.2% | 94.4% | 112.6% |
| 10q # del | 5 | 20.0% | 88.9% | 108.9% | 5 | 14.3% | 94.1% | 108.4% |
| 10q # targets | 89 | 66.7% | 44.4% | 111.1% | 89 | 64.3% | 53.3% | 117.6% |
| 3p # Abn. | 47 | 6.7% | 100.0% | 106.7% | 2 | 69.2% | 37.5% | 106.7% |
| 3p # Poly | 4 | 66.7% | 77.8% | 144.4% | 4 | 58.3% | 82.4% | 140.7% |
| 3p # del | 3 | 46.7% | 83.3% | 130.0% | 3 | 20.0% | 93.8% | 113.8% |
| 3p # targets | 84 | 86.7% | 33.3% | 120.0% | 84 | 86.7% | 37.5% | 124.2% |
| LAV # Abn. | 5 | 60.0% | 94.1% | 154.1% | 5 | 50.0% | 94.1% | 144.1% |
| Total 10q | 17 | 33.3% | 94.4% | 127.8% | 17 | 9.1% | 94.4% | 103.5% |
| Total 10q (- | 16 | 33.3% | 100.0% | 133.3% | 16 | 9.1% | 100.0% | 109.1% |
| Abn.) | | | | | | | | |
| Total 10q (- | 0.06 | 46.7% | 88.9% | 135.6% | 0.06 | 38.5% | 88.9% | 127.4% |
| Abn.)/10q # | | | | | | | | |
| targets | | | | | | | | |
| Total | 0.06 | 53.3% | 83.3% | 136.7% | 0.06 | 50.0% | 83.3% | 133.3% |
| 10q/10q # | | | | | | | | |
| targets | | | | | | | | |
| Total 3p | 7 | 86.7% | 44.4% | 131.1% | 7 | 85.7% | 44.4% | 130.2% |
| Total 3p (- | 6 | 86.7% | 72.2% | 158.9% | 6 | 81.8% | 76.5% | 158.3% |
| Abn.) | | | | | | | | |
| Total 3p (- | 0.05 | 80.0% | 88.9% | 168.9% | 0.05 | 75.0% | 88.9% | 163.9% |
| Abn.)/3p # | | | | | | | | |
| targets | | | | | | | | |
| Total 3p, 10q | 38 | 40.0% | 88.9% | 128.9% | 9 | 81.8% | 38.9% | 120.7% |
| Total 3p, 10q | 15 | 60.0% | 77.8% | 137.8% | 15 | 50.0% | 77.8% | 127.8% |
| (-Abn.) | | | | | | | | |
| Total 3p/3p # | 0.06 | 86.7% | 55.6% | 142.2% | 0.06 | 83.3% | 55.6% | 138.9% |
| targets | | | | | | | | |

Table 9: Sensitivity and specificity results for target parameters (single parameter).
Cutoff = the number of target cells exhibiting a certain parameter which are required for determination of subject's clinical condition (healthy or having lung cancer). Note that when the parameter used includes an equation (e.g., a ratio of cells having a certain parameter out of the cells which can be scored using another parameter) the cutoff is the result of such equation, e.g., a number between 0 to 1.

The results demonstrate that the promising parameters are Total 3p (−Abn.) and Total 3p (−Abn)/3p # targets. Both parameters yield higher Youden's Index than LAV, although the results are not significant (p-value=0.948 and 0.487, respectively).

TABLE 10
Sensitivity and specificity results for area parameters (single parameter)

| | All Data | | | | Without Outliers | | | |
|---|---|---|---|---|---|---|---|---|
| | | | | Youden's | | | | Youden's |
| Parameter | Cutoff | Sensitivity | Specificity | Index | Cutoff | Sensitivity | Specificity | Index |
| 10q # Abn. | 0.39% | 78.6% | 77.8% | 156.3% | 0.39% | 75.0% | 82.4% | 157.4% |
| 10q # Poly | 1.22% | 50.0% | 100.0% | 150.0% | 0.59% | 54.5% | 83.3% | 137.9% |
| 10q # del | 0.90% | 71.4% | 72.2% | 143.7% | 0.90% | 71.4% | 72.2% | 143.7% |
| 3p # Abn. | 0.95% | 71.4% | 55.6% | 127.0% | 0.95% | 66.7% | 58.8% | 125.5% |
| 3p # Poly | 0.71% | 64.3% | 77.8% | 142.1% | 0.71% | 61.5% | 82.4% | 143.9% |
| 3p # del | 2.25% | 42.9% | 88.9% | 131.7% | 2.25% | 38.5% | 88.9% | 127.4% |
| LAV # Abn. | 7.00% | 50.0% | 93.8% | 143.8% | 7.00% | 46.2% | 93.8% | 139.9% |
| Total 10q | 1.18% | 92.9% | 66.7% | 159.5% | 1.18% | 92.9% | 66.7% | 159.5% |
| Total 10q (- | 2.72% | 64.3% | 88.9% | 153.2% | 1.18% | 84.6% | 66.7% | 151.3% |
| Abn.) | | | | | | | | |
| Total 3p | 2.81% | 92.9% | 50.0% | 142.9% | 2.81% | 92.3% | 52.9% | 145.2% |
| Total 3p (- | 2.54% | 64.3% | 77.8% | 142.1% | 2.54% | 58.3% | 77.8% | 136.1% |
| Abn.) | | | | | | | | |
| Total 3p, | 5.58% | 78.6% | 77.8% | 156.3% | 5.58% | 78.6% | 77.8% | 156.3% |
| 10q | | | | | | | | |
| Total 3p, | 4.12% | 78.6% | 77.8% | 156.3% | 4.12% | 78.6% | 77.8% | 156.3% |
| 10q (-Abn.) | | | | | | | | |

Table 10: Sensitivity and specificity results for area parameters (single parameter).
Cutoff = the percentage of cells exhibiting a certain parameter which are required for determination of subject's clinical condition (healthy or having lung cancer).

The results shown demonstrate that the parameter which yields the highest Youden's Index is Total 10q, though it is not significantly higher than LAV (p-value=0.660).

The following Tables present results of sensitivity and specificity based on a single parameter. For each parameter the cutoff that yields the maximal sensitivity given that specificity is fixed at least 80% is presented. For all parameters, the decision rule is to classify a subject as having lung cancer if his or her measured value is above the cutoff. Results are presented for all data and for data excluding outliers. For ease of presentation only the parameters for which the sensitivity was higher than 50% were included. Table 11, presents results for Target parameters and Table 12 for Area parameters.

TABLE 11
Sensitivity results for target parameters (single parameter),
specificity ≧80%

| | All Data | | Without Outliers | |
|---|---|---|---|---|
| Parameter | Cutoff | Sensitivity | Cutoff | Sensitivity |
| LAV # Abn. | 5 | 60.0% | | |
| Total 10q/10q # targets | 0.06 | 53.3% | | |
| Total 3p (-Abn.)/3p # targets | 0.05 | 80.0% | 0.05 | 75.0% |
| Total 3p, 10q (-Abn.) | 19 | 53.3% | | |
| 3p # Poly | | | 4 | 58.3% |

Table 11: Sensitivity results for target parameters (single parameter) when the specificity is fixed at ≧80%. Cutoff = the number of target cells exhibiting a certain parameter which are required for determination of subject's clinical condition (healthy or having lung cancer). Note that when the parameter used includes an equation (e.g., a ratio of cells having a certain parameter out of the cells which can be scored using another parameter) the cutoff is the result of such equation, e.g., a number between 0 to 1.

The results show that when specificity is fixed at 80%, using the ‘Total 3p (−Abn.)/3p # targets’ parameter, a sensitivity of 80% can be achieved.

TABLE 12
Sensitivity results for area parameters (single parameter),
specificity ≧80%

| | All Data | | Without Outliers | |
|---|---|---|---|---|
| Parameter | Cutoff | Sensitivity | Cutoff | Sensitivity |
| 10q # Abn. | 0.46% | 64.3% | 0.39% | 75.0% |
| Total 10q | 2.18% | 71.4% | 2.18% | 71.4% |
| Total 10q (-Abn.) | 2.72% | 64.3% | 2.72% | 61.5% |
| 10q # Poly | | | 0.59% | 54.5% |
| 3p # Poly | | | 0.71% | 61.5% |

Table 12: Sensitivity results for area parameters (single parameter) when the specificity is fixed on ≧80%. Cutoff = the percentage of cells exhibiting a certain parameter which are required for determination of subject's clinical condition (healthy or having lung cancer).

The results show that when specificity is fixed at 80%, using the Total 10q′ parameter, a sensitivity of 71.4% can be achieved.

The following Tables present results of sensitivity and specificity analysis based on a single parameter and using only subjects whose number of targets was above 50 in the target parameters (i.e., which included more than 50 relevant cells for target scan of the sample). For each parameter the cutoff that yields the maximal sensitivity given that specificity is fixed at least at 80% is presented. For all parameters, the decision rule is to classify a subject as having lung cancer if his or her measured value is above the cutoff. For ease of presentation only the parameters for which the sensitivity was higher than 50% and repeat the results obtained using the entire data are presented. Table 13 presents results for Target parameters (using target scan) and Table 14 for Area parameters (using area scan).

TABLE 13
Sensitivity results for target parameters (single parameter) for data
excluding number of targets <50, specificity ≧80%

| | Data Excluding # of | | | |
|---|---|---|---|---|
| | Targets <50 | | Original Data | |
| Parameter | Cutoff | Sensitivity | Cutoff | Sensitivity |
| 3p # Poly | 6 | 58.3% | | |
| LAV # Abn. | 5 | 75.0% | 5 | 60.0% |
| Total 10q (- | 0.06 | 58.3% | | |
| Abn.)/10q # | | | | |
| targets | | | | |
| Total 10q/10q # | 0.06 | 58.3% | 0.06 | 53.3% |
| targets | | | | |
| Total 3p (-Abn.) | 12 | 58.3% | | |
| Total 3p (- | 0.04 | 91.7% | 0.05 | 80.0% |
| Abn.)/3p # targets | | | | |
| Total 3p, 10q (- | 19 | 66.7% | 19 | 53.3% |
| Abn.) | | | | |

Table 13 Sensitivity results for target parameters (single parameter) for data excluding samples with number of targets <50, with a fixed specificity of ≧80%. Cutoff = the number of target cells exhibiting a certain parameter which are required for determination of subject's clinical condition (healthy or having lung cancer). Note that when the parameter used includes an equation (e.g., a ratio of cells having a certain parameter out of the cells which can be scored using another parameter) the cutoff is the result of such equation, e.g., a number between 0 to 1.

The results show that after excluding sputum samples which included less that 50 target cells/sputum sample (which resulted in the exclusion of 6 subjects from the analysis) the sensitivity of the various parameters was improved. For example, using the ‘Total 3p (−Abn.)/3p # targets’ parameter, a sensitivity of 91.7% can be achieved when the specificity is fixed at 80% (compared with 80% sensitivity when using samples including less that 50 target cells.

TABLE 14
Sensitivity results for area parameters (single parameter) for data
excluding # of targets <50, specificity ≧80%

| | Data Excluding # of Targets <50 | | Original Data | |
|---|---|---|---|---|
| Parameter | Cutoff | Sensitivity | Cutoff | Sensitivity |
| 10q # Abn. | 0.46% | 63.6% | 0.46% | 64.3% |
| 10q # Poly | 1.22% | 63.6% | | |
| 10q # del | 1.59% | 54.5% | | |
| LAV # Abn. | 7.00% | 54.5% | | |
| Total 10q | 2.18% | 81.8% | 2.18% | 71.4% |
| Total 10q (-Abn.) | 2.72% | 72.7% | 2.72% | 64.3% |
| Total 3p (-Abn.) | 3.10% | 54.5% | | |
| Total 3p, 10q | 9.25% | 54.5% | | |
| Total 3p, | 5.66% | 54.5% | | |
| 10q (-Abn.) | | | | |

Table 14: Sensitivity results for area parameters (single parameter) for data excluding samples with # of targets <50 (The same samples that were excluded in target scan based on the number of targets), when the specificity is fixed ≧80%. Cutoff = the percentage of cells exhibiting a certain parameter which are required for determination of subject's clinical condition (healthy or having lung cancer).

The results show that when excluding sputum samples which include less than 50 cells (and thus excluding 6 subjects from analysis) the sensitivity of the various parameters is improved. For example, using the ‘Total 10q’ parameter, a sensitivity of 81.8% can be achieved when the specificity is fixed at 80% (compared with 71.4% sensitivity when sputum samples with less than 50 cells are included).

Looking at all of the results obtained based on a single parameter, the most promising predictors for detection of lung cancer are ‘Total 3p (−Abn.)/3p # targets’ among the target parameters and ‘Total 10q’ among the area parameters. Removing observations with low number of targets (less than 50) in the target parameters improves the prediction of the models.

Two Parameters—The current section presents the sensitivity/specificity results based on two parameters. Since analyzing the data without outliers had little effect on the single parameters results, this analysis was performed on the entire data only. All possible pairwise combinations of parameters were analyzed in order to detect the pairs that yield the highest sensitivity and specificity sum. As described hereinabove, two types of decision rules were employed in order to select the optimal cutoffs. These were:

Decision rule 1: A subject is classified as having lung cancer if both values obtained for the two parameters are above their respective cutoffs.

Decision rule 2: A subject is classified as having lung cancer if at least one of the two parameters is above its respective cutoff.

Table 15 presents the combinations that yielded Youden's Index higher than 170% using decision rule 1 and Table 16 presents the combination that yielded Youden's Index higher than 170% using decision rule 2.

TABLE 15
Sensitivity and specificity results based on a combination of
two parameters and using decision rule 1

| | | | | | | Youden's |
|---|---|---|---|---|---|---|
| Predictor 1 | Predictor 2 | Cutoff 1 | Cutoff 2 | Sensitivity | Specificity | Index |
| Target - Total | Area - Total 10q | 6 | 1.2% | 85.7% | 88.9% | 174.6% |
| 3p (-Abn.) | Area - Total 10q | 6 | 1.2% | 85.7% | 88.9% | 174.6% |
| | (-Abn.) | | | | | |
| | Area - Total 3p, | 6 | 4.1% | 78.6% | 94.4% | 173.0% |
| | 10q (-Abn.) | | | | | |
| Target - Total | Target - Total 3p | 0.04 | 3 | 86.7% | 83.3% | 170.0% |
| 3p (-Abn.)/3p | (-Abn.) | | | | | |
| # targets | | | | | | |

Table 15: Sensitivity and specificity results based on a combination of two parameters and using decision rule 1.
Cutoff = the absolute number (for target parameters) or percentage (for area scan parameters) of cells required for determination of subject's clinical condition (healthy or having lung cancer). Cutoff (for target scan) = the number of target cells exhibiting a certain parameter which are required for determination of subject's clinical condition (healthy or having lung cancer). Note that when the parameter used includes an equation (e.g., a ratio of cells having a certain parameter out of the cells which can be scored using another parameter) the cutoff is the result of such equation, e.g., a number between 0 to 1.
Cutoff (for area scan) = the percentage of cells exhibiting a certain parameter which are required for determination of subject's clinical condition (healthy or having lung cancer).

Table 15 shows that the combinations of parameters that yields the highest Youden's Index are:

1. Target—Total 3p (−Abn.) and Area—Total 10q;

2. Target—Total 3p (−Abn.) and Area—Total 10q (−Abn.).

The difference in the prediction obtained from the (single) LAV parameter (using Target statistics) and the above two combinations is not statistically significant (p-value=0.326).

TABLE 16
Sensitivity and specificity results based on a combination of
two parameters and using decision rule 2

| | | | | | | Youden's |
|---|---|---|---|---|---|---|
| Predictor 1 | Predictor 2 | Cutoff 1 | Cutoff 2 | Sensitivity | Specificity | Index |
| Area - 10q # | Area - 10q # Abn. | 1.22% | 0.7% | 85.7% | 88.9% | 174.6% |
| Poly | | | | | | |
| Target - | Area - 10q # Abn. | 0.05 | 0.8% | 92.9% | 83.3% | 176.2% |
| Total 3p (- | Area - 10q # Poly | 0.06 | 1.2% | 78.6% | 94.4% | 173.0% |
| Abn.)/3p # | Area - 10q # del | 0.05 | 2.8% | 85.7% | 88.9% | 174.6% |
| targets | Area - 3p # Abn. | 0.05 | 6.5% | 85.7% | 88.9% | 174.6% |
| | Area - Total 10q | 0.05 | 4.5% | 85.7% | 88.9% | 174.6% |
| | Area - Total 10q (- | 0.05 | 3.4% | 85.7% | 88.9% | 174.6% |
| | Abn.) | | | | | |

Table 16: Sensitivity and specificity results based on a combination of two parameters and using decision rule 2.
Cutoff (for target scan) = the number of target cells exhibiting a certain parameter which are required for determination of subject's clinical condition (healthy or having lung cancer). Note that when the parameter used includes an equation (e.g., a ratio of cells having a certain parameter out of the cells which can be scored using another parameter) the cutoff is the result of such equation, e.g., a number between 0 to 1.
Cutoff (for area scan) = the percentage of cells exhibiting a certain parameter which are required for determination of subject's clinical condition (healthy or having lung cancer).

Table 16 demonstrates that the combination of parameters that yields the higher Youden's Index is ‘Target—Total 3p (−Abn.)/3p # targets and Area—10q # Abn’.

The difference in the prediction obtained from the (single) LAV parameter (using Target statistics) and the above three combinations is not statistically significant (p-value=0.326).

The following Tables present results of the two parameters analysis repeated for cutoffs that yield the maximal sensitivity given that specificity is fixed at least at 80%-once for the entire original data and once using only subjects in which the number of targets (relevant cells in the sample) was above 50 in the target parameters. Table 17 presents the combinations that yielded sensitivity higher than 85% using decision rule 1 and Table 18 presents the combination that yielded sensitivity higher than 85% using decision rule 2

TABLE 17
Sensitivity results based on a combination of two parameters and
using decision rule 1, specificity ≧80%

| | | Data Excluding | | | | | |
|---|---|---|---|---|---|---|---|
| | | # of Targets < 50 | | | Original Data | | |
| Predictor 1 | Predictor 2 | Cutoff 1 | Cutoff 2 | Sensitivity | Cutoff 1 | Cutoff 2 | Sensitivity |
| Area - Total 3p, | Area - 10q # Abn. | 4% | 0.16% | 90.9% | | | |
| 10q (-Abn.) | | | | | | | |
| Target - 10q # | Area - Total 10q | 1 | 1.33% | 90.9% | | | |
| Poly | Area - Total 10q | 1 | 1.20% | 90.9% | | | |
| | (-Abn.) | | | | | | |
| | Area - Total 3p, | 1 | 4.12% | 90.9% | | | |
| | 10q (-Abn.) | | | | | | |
| Target - 10q # | Area - Total 3p, | 58 | 4.12% | 90.9% | | | |
| targets | 10q (-Abn.) | | | | | | |
| Target - 3p # | Area - Total 10q | 3 | 1.18% | 90.9% | | | |
| Poly | Area - Total 10q | 3 | 1.15% | 90.9% | | | |
| | (-Abn.) | | | | | | |
| | Area - Total 3p, | 3 | 2.25% | 90.9% | | | |
| | 10q (-Abn.) | | | | | | |
| Target - 3p # | Area - Total 10q | 117 | 1.15% | 90.9% | | | |
| targets | (-Abn.) | | | | | | |
| | Area - Total 3p, | 84 | 4.12% | 90.9% | | | |
| | 10q (-Abn.) | | | | | | |
| Target - Total | Area - Total 3p, | 1 | 4.12% | 90.9% | | | |
| 10q | 10q (-Abn.) | | | | | | |
| Target - Total | Area - Total 3p, | 1 | 4.12% | 90.9% | | | |
| 10q (-Abn.) | 10q (-Abn.) | | | | | | |
| Target - Total | Area - Total 3p, | 0.01 | 4.12% | 90.9% | | | |
| 10q (-Abn.)/10q | 10q (-Abn.) | | | | | | |
| # targets | | | | | | | |
| Target - Total | Area - Total 3p, | 0.02 | 4.12% | 90.9% | | | |
| 10q/10q # | 10q (-Abn.) | | | | | | |
| targets | | | | | | | |
| Target - Total | Area - Total 3p, | 7 | 4.12% | 90.9% | | | |
| 3p | 10q (-Abn.) | | | | | | |
| Target - Total | Area - Total 10q | 6 | 1.18% | 100.0% | 6 | 1.18% | 85.7% |
| 3p (-Abn.) | Area - Total 10q | 6 | 1.15% | 100.0% | 6 | 1.18% | 85.7% |
| | (-Abn.) | | | | | | |
| | Area - Total 3p, | 6 | 4.12% | 90.9% | | | |
| | 10q (-Abn.) | | | | | | |
| Target - Total | Area - 10q # Abn. | 0.04 | 0.00% | 90.9% | 0.04 | 0.00% | 85.7% |
| 3p (-Abn.)/3p # | Area - Total 10q | 0.04 | 1.18% | 90.9% | 0.04 | 1.06% | 85.7% |
| targets | Area - Total 10q | 0.04 | 1.15% | 90.9% | 0.04 | 0.85% | 85.7% |
| | (-Abn.) | | | | | | |
| | Area - Total 3p | 0.04 | 1.60% | 90.9% | | | |
| | Area - Total 3p, | 0.04 | 3.75% | 90.9% | | | |
| | 10q | | | | | | |
| | Target - 10q # | 0.04 | 58 | 91.7% | | | |
| | targets | | | | | | |
| | Target - 3p # | 0.04 | 0 | 91.7% | | | |
| | Abn. | | | | | | |
| | Target - 3p # | 0.04 | 84 | 91.7% | | | |
| | targets | | | | | | |
| | Target - Total 10q | 0.04 | 0 | 91.7% | | | |
| | Target - Total 10q | 0.04 | 0 | 91.7% | | | |
| | (-Abn.) | | | | | | |
| | Target - Total 10q | 0.04 | 0 | 91.7% | | | |
| | (-Abn.)/10q # | | | | | | |
| | targets | | | | | | |
| | Target - Total | 0.04 | 0 | 91.7% | | | |
| | 10q/10q # targets | | | | | | |
| | Target - Total 3p | 0.04 | 7 | 91.7% | | | |
| | Target - Total 3p | 0.02 | 6 | 100.0% | 0.04 | 3 | 86.7% |
| | (-Abn.) | | | | | | |
| Target - Total | Target - Total 3p | 9 | 0.357 | 91.7% | | | |
| 3p, 10q | (-Abn.)/3p # | | | | | | |
| | targets | | | | | | |
| Target - Total | Area - Total 3p, | 8 | 4.12% | 90.9% | | | |
| 3p, 10q (-Abn.) | 10q (-Abn.) | | | | | | |
| | Target - Total 3p | 6 | 0.357 | 91.7% | | | |
| | (-Abn.)/3p # | | | | | | |
| | targets | | | | | | |
| Target - Total | Target - Total 3p | 0.06 | 6 | 91.7% | | | |
| 3p/3p # targets | (-Abn.) | | | | | | |
| | Target - Total 3p | 0.06 | 0.357 | 91.7% | 0.06 | 0.357 | 86.7% |
| | (-Abn.)/3p # | | | | | | |
| | targets | | | | | | |

Table 17: Sensitivity results based on a combination of two parameters and using decision rule 1, specificity ≧80%.
Cutoff (for target scan) = the number of target cells exhibiting a certain parameter which are required for determination of subject's clinical condition (healthy or having lung cancer). Note that when the parameter used includes an equation (e.g., a ratio of cells having a certain parameter out of the cells which can be scored using another parameter) the cutoff is the result of such equation, e.g., a number between 0 to 1.
Cutoff (for area scan) = the percentage of cells exhibiting a certain parameter which are required for determination of subject's clinical condition (healthy or having lung cancer).

Table 17 demonstrates that excluding samples which include less than 50 cells (resulted in exclusion of 6 samples) results in higher sensitivity. The highest sensitivity (100%, when excluding observations with less than 50 targets) was obtained when combining ‘Target—Total 3p (−Abn.)/3p # targets’ with ‘Target—Total 3p (−Abn.)’.

TABLE 18
Senstivity results based on a combination of two parameters and
using decision rule 2, specificity ≧80%

| | | Data Excluding | | | | | |
|---|---|---|---|---|---|---|---|
| | | # of Targets < 50 | | | Original Data | | |
| Predictor 1 | Predictor 2 | Cutoff 1 | Cutoff 2 | Sensitivity | Cutoff 1 | Cutoff 2 | Sensitivity |
| Area - 10q # | Area - 10q # Abn. | 0.01 | 0.74% | 90.9% | 0.01 | 0.74% | 85.7% |
| Poly | | | | | | | |
| Area - Total | Area - 10q # Poly | 0.02 | 1.22% | 90.9% | | | |
| 10q | | | | | | | |
| Area - Total | Area - 10q # Abn. | 0.03 | 0.53% | 90.9% | 0.03 | 0.53% | 85.7% |
| 3p (-Abn.) | Area - Total 10q | 0.03 | 2.18% | 90.9% | | | |
| Target - 3p # | Area - Total 10q (-Abn.) | 4 | 3.23% | 90.9% | | | |
| del | | | | | | | |
| Target - Total | Area - 10q # Poly | 0.07 | 0.59% | 90.9% | | | |
| 10q (- | | | | | | | |
| Abn.)/10q # | | | | | | | |
| targets | | | | | | | |
| Target - Total | Area - 10q # Abn. | 0.05 | 0.81% | 90.9% | 0.05 | 0.77% | 92.9% |
| 3p (-Abn.)/3p | Area - 10q # del | 0.04 | 2.77% | 100.0% | 0.05 | 2.77% | 85.7% |
| # targets | Area - 3p # Abn. | 0.04 | 6.54% | 100.0% | 0.05 | 6.54% | 85.7% |
| | Area - 3p # Poly | 0.04 | 7.33% | 90.9% | | | |
| | Area - 3p # del | 0.04 | 5.93% | 90.9% | | | |
| | Area - Total 10q | 0.04 | 4.48% | 100.0% | 0.05 | 4.48% | 85.7% |
| | Area - Total 10q (-Abn.) | 0.04 | 3.35% | 100.0% | 0.05 | 3.35% | 85.7% |
| | Area - Total 3p | 0.04 | 14.83% | 90.9% | | | |
| | Area - Total 3p (-Abn.) | 0.04 | 8.60% | 90.9% | | | |
| | Area - Total 3p, 10q | 0.04 | 18.62% | 90.9% | | | |
| | Area - Total 3p, 10q (-Abn.) | 0.04 | 12.36% | 90.9% | | | |
| | Target - 10q # Abn. | 0.04 | 12 | 91.7% | | | |
| | Target - 10q # Poly | 0.04 | 67 | 91.7% | | | |
| | Target - 10q # del | 0.04 | 13 | 91.7% | | | |
| | Target - 10q # targets | 0.04 | 767 | 91.7% | | | |
| | Target - 3p # Abn. | 0.04 | 60 | 91.7% | | | |
| | Target - 3p # Poly | 0.04 | 45 | 91.7% | | | |
| | Target - 3p # del | 0.04 | 31 | 91.7% | | | |
| | Target - 3p # targets | 0.04 | 732 | 91.7% | | | |
| | Target - Total 10q | 0.04 | 91 | 91.7% | | | |
| | Target - Total 10q (-Abn.) | 0.04 | 79 | 91.7% | | | |
| | Target - Total 10q (- | 0.04 | 0.574 | 91.7% | | | |
| | Abn.)/10q # targets | | | | | | |
| | Target - Total 10q/10q # | 0.04 | 0.574 | 91.7% | | | |
| | targets | | | | | | |
| | Target - Total 3p | 0.04 | 95 | 91.7% | | | |
| | Target - Total 3p (-Abn.) | 0.04 | 45 | 91.7% | | | |
| Target - Total | Target - Total 3p (-Abn.)/3p | 135 | 0.357 | 91.7% | | | |
| 3p, 10q | # targets | | | | | | |
| Target - Total | Target - Total 3p (-Abn.)/3p | 117 | 0.357 | 91.7% | | | |
| 3p, 10q (- | # targets | | | | | | |
| Abn.) | | | | | | | |
| Target - Total | Target - Total 3p (-Abn.)/3p | 0.46 | 0.357 | 91.7% | | | |
| 3p/3p # | # targets | | | | | | |
| targets | | | | | | | |

Table 18: Sensitivity results based on a combination of two parameters and using decision rule 2, specificity ≧80%
Cutoff (for target scan) = the number of target cells exhibiting a certain parameter which are required for determination of subject's clinical condition (healthy or having lung cancer). Note that when the parameter used includes an equation (e.g., a ratio of cells having a certain parameter out of the cells which can be scored using another parameter) the cutoff is the result of such equation, e.g., a number between 0 to 1.
Cutoff (for area scan) = the percentage of cells exhibiting a certain parameter which are required for determination of subject's clinical condition (healthy or having lung cancer).

Table 18 shows that excluding samples which include less than 50 target cells (which results in exclusion of 6 samples) generates better sensitivity results. The highest sensitivity (100%, when excluding observations with less than 50 targets) was obtained when combining ‘Target—Total 3p (−Abn.)/3p # targets’ with one of the following Area parameters: ‘10q # del’, ‘3p # Abn.’, ‘Total 10q’ or ‘Total 10q (−Abn.)’.

Looking at all of the results obtained based on a combination of two parameters, the most promising pair of predictors for detection of lung cancer is a combination of ‘Total 3p (−Abn.)/3p # targets’ among the target parameters and with another parameter. Once again, removing observations with low number of targets in the target parameters improves the prediction of the models. As expected, using a combination of two parameters generates better results than using only one parameter.

Power Analysis—The following Table presents the results of the power analysis.

TABLE 19
Number of subjects required to achieve at least 80%

| | Constant | | | | |
|---|---|---|---|---|---|
| True Sensitivity | 70% | 75% | 80% | 85% | 90% |
| 80% | 155 | 553 | | | |
| 85% | 61 | 132 | 466 | | |
| 90% | 31 | 54 | 107 | 356 | |
| 95% | 22 | 27 | 41 | 75 | 231 |

Table 19.

Table 19 shows that if, for example, the assumption is that the “true sensitivity” of the procedure is 90%, then a sample of 54 diseased subjects will provide 80% power to test the hypothesis that the sensitivity obtained in the sample is higher than 75%. To test the hypothesis that the sensitivity obtained is higher than 80%, a sample of 107 diseased subjects would be required in order to achieve 80% power.

List of Outliers—The following Table presents a listing of all the outliers that were excluded from the ‘Without Outliers’ analysis. A value was considered to be an outlier if it was higher than 1.5 times the inter-quartile range above the 3^{rd} quartile.

TABLE 20
Listing of outliers by parameter

| Statistics | Diagnostic | Case # | Predictor | Value |
|---|---|---|---|---|
| Target | Control | IS-103 | 3p # Abn. | 29 |
| Target | Control | IS-12 | 10q # Abn. | 11 |
| Target | Control | IS-24 | 10q # targets | 711 |
| Target | Control | IS-24 | 3p # Abn. | 47 |
| Target | Control | IS-24 | 3p # targets | 732 |
| Target | Control | IS-41 | 10q # targets | 767 |
| Target | Control | IS-41 | 3p # Poly | 32 |
| Target | Control | IS-41 | 3p # targets | 720 |
| Target | Control | IS-7 | 10q # del | 13 |
| Target | Control | IS-77 | 10q # targets | 575 |
| Target | Lung Cancer | IS-1 | 10q # Poly | 20 |
| Target | Lung Cancer | IS-1 | 3p # del | 31 |
| Target | Lung Cancer | IS-1 | LAV # Abn. | 33 |
| Target | Lung Cancer | IS-1 | Total 10q (-Abn.) | 29 |
| Target | Lung Cancer | IS-1 | Total 3p, 10q (-Abn.) | 64 |
| Target | Lung Cancer | IS-14 | 10q # Poly | 22 |
| Target | Lung Cancer | IS-14 | 3p # Abn. | 24 |
| Target | Lung Cancer | IS-14 | Total 10q (-Abn.) | 26 |
| Target | Lung Cancer | IS-150 | Total 3p (-Abn.)/3p | 0.2857143 |
| | | | # targets | |
| Target | Lung Cancer | IS-150 | Total 3p/3p # targets | 0.2857143 |
| Target | Lung Cancer | IS-2 | 3p # del | 16 |
| Target | Lung Cancer | IS-2 | Total 3p (-Abn.)/3p | 0.1811024 |
| | | | # targets | |
| Target | Lung Cancer | IS-43 | 3p # Abn. | 60 |
| Target | Lung Cancer | IS-43 | 3p # Poly | 35 |
| Target | Lung Cancer | IS-43 | Total 3p | 95 |
| Target | Lung Cancer | IS-43 | Total 3p, 10q | 98 |
| Target | Lung Cancer | IS-43 | Total 3p/3p # targets | 0.3518519 |
| Target | Lung Cancer | IS-81 | 10q # Abn. | 12 |
| Target | Lung Cancer | IS-81 | 10q # Poly | 67 |
| Target | Lung Cancer | IS-81 | 10q # del | 12 |
| Target | Lung Cancer | IS-81 | 10q # targets | 527 |
| Target | Lung Cancer | IS-81 | 3p # Poly | 27 |
| Target | Lung Cancer | IS-81 | LAV # Abn. | 36 |
| Target | Lung Cancer | IS-81 | Total 10q | 91 |
| Target | Lung Cancer | IS-81 | Total 10q (-Abn.) | 79 |
| Target | Lung Cancer | IS-81 | Total 3p, 10q | 135 |
| Target | Lung Cancer | IS-81 | Total 3p, 10q (-Abn.) | 117 |
| Target | Lung Cancer | IS-82 | 10q # Poly | 49 |
| Target | Lung Cancer | IS-82 | 3p # Poly | 45 |
| Target | Lung Cancer | IS-82 | LAV # Abn. | 62 |
| Target | Lung Cancer | IS-82 | Total 10q | 50 |
| Target | Lung Cancer | IS-82 | Total 10q (-Abn.) | 50 |
| Target | Lung Cancer | IS-82 | Total 10q (-Abn.)/10q | 0.5747126 |
| | | | # targets | |
| Target | Lung Cancer | IS-82 | Total 10q/10q | 0.5747126 |
| | | | # targets | |
| Target | Lung Cancer | IS-82 | Total 3p (-Abn.)/3p | 0.4455446 |
| | | | # targets | |
| Target | Lung Cancer | IS-82 | Total 3p, 10q | 96 |
| Target | Lung Cancer | IS-82 | Total 3p, 10q (-Abn.) | 95 |
| Target | Lung Cancer | IS-82 | Total 3p/3p # targets | 0.4554455 |
| Area | Control | IS-12 | 3p # Abn. | 0.0654 |
| Area | Control | IS-12 | Total 3p | 0.1203 |
| Area | Control | IS-41 | 3p # Poly | 0.0526 |
| Area | Control | IS-8 | 10q # Abn. | 0.028 |
| Area | Lung Cancer | IS-1 | 10q # Abn. | 0.0321 |
| Area | Lung Cancer | IS-10 | 10q # Abn. | 0.0333 |
| Area | Lung Cancer | IS-14 | 3p # Abn. | 0.0623 |
| Area | Lung Cancer | IS-14 | 3p # del | 0.0593 |
| Area | Lung Cancer | IS-14 | Total 3p | 0.1483 |
| Area | Lung Cancer | IS-14 | Total 3p (-Abn.) | 0.086 |
| Area | Lung Cancer | IS-155 | 3p # Abn. | 0.0773 |
| Area | Lung Cancer | IS-42 | 10q # Poly | 0.0335 |
| Area | Lung Cancer | IS-81 | 10q # Poly | 0.0667 |
| Area | Lung Cancer | IS-81 | Total 10q (-Abn.) | 0.0892 |
| Area | Lung Cancer | IS-82 | 10q # Poly | 0.041 |
| Area | Lung Cancer | IS-82 | 3p # Poly | 0.0733 |
| Area | Lung Cancer | IS-82 | LAV # Abn. | 0.62 |
| Area | Lung Cancer | IS-82 | Total 3p (-Abn.) | 0.0733 |

Table 21.

### Example 4

### Detection of Lung Cancer in Induced Sputum by Combined Analysis of Morphology and Fish

Evaluation of induced sputum samples from 71 subjects: 14 patients diagnosed with advanced stage (stage III-IV) lung cancer, 15 patients diagnosed with early stage lung cancer (stage I), 32 high-risk volunteers (heavy smokers and 10 healthy non-smoking controls. The local ethical review committee approved the study and informed consent was obtained from all patients.

Materials and Methods

Sputum production and processing—Induced sputum production was performed by saline inhalation with a nebulizer. In the cancer patients who were resected or were referred to bronchoscopy, induced sputum samples were collected before the invasive procedure. Following inhalation, patients and controls were instructed to cough into a container that was filled with Sacommano's fixative (90% alcohol, 5% acetic acid and 5% polyethylene glycol). Sputum samples were centrifuged and/or filtered through gauze and the sediment was used to prepare at least 10 cytospins (cytocentrifugation samples) on positively charged glass slides using a SHANDON-Cytosine 2 cytocentrifuge (Pittsburgh, Pa.). Slides were fixed in 95% ethanol.

Morphological staining—Slides were stained with Papanicolaou stain according to standard protocols. The slides containing the stained cells were subject to morphological analysis in a Bright field mode using the Bio View Duet.

Destaining and pretreatment—Following morphological evaluation of the stained cells the slides were immersed in Xylene until the removal of the coverslip. The slides were then washed in an Ethanol series of 100%, 95% and 70% and immersed for 10 minutes in 1.5% Acid Alcohol at room temperature. Slides were then washed in running water and immersed for 60 minutes in 2×SSC at 37° C.

Prior to FISH, slides were digested in 10 mM HCl/0.05% digestion enzyme (BioView Ltd. Rehovot, Israel) for approximately 18 minutes at 37° C. Then the slides were washed for 5 minutes in 1×PBS, fixed for 5 minutes in 1% formaldehyde/PBS, washed once for 5 minutes in 1×PBS and dehydrated in an ice-cold ethanol series (70, 80, 100%).

FISH—A 3-color FISH assay was performed using directly labeled BAC probes for 3p22.1 (GenBank Accession No. AC104186), 10q22-23 (GenBank Accession No. AC068139) combined with commercial centromeric probes for chromosome 10 (CEP 10) (Vysis Downers grove, Ill.). It should be noted that FISH can be performed using multiple FISH probes, each corresponding to a distinct color, e.g., 2-color FISH probes, 3-color FISH probes, 4-colors FISH probes and more. A further description of the BAC probes is provided in Example 1, above.

The BAC probe located at 10q22.2-q23.1 was labeled with Spectrum Red dUTP (Vysis, Downers Grove, Ill.) (5). The chromosomal location of the clone was confirmed by FISH on a normal metaphase spread. The BAC clone for 3p22.1 was labeled with Spectrum Green dUTP (Vysis, Downers Grove, Ill.) according to the manufacturer's directions. Localization of the BAC clone on chromosome 3 was confirmed by using normal metaphase FISH. The centromeric 10 probe (CEP 10), available from Vysis (Downers Grove, Ill.) is fluorescently-labeled with Spectrum Aqua. One hundred nanograms of each labeled probe (i.e., of the 10q22.2-q23.1 and 3p22.1 BAC probes was mixed with an equal quantity of human Cot-1 DNA (Life Technologies, Rockville, Md.) in 10 μl of LSI hybridization buffer (Vysis) and mounted on a slide together with 1 μl of CEP 10. Hybridization and post-washing were performed as described previously (5). Counterstaining of nuclei was performed with DAPI and evaluated under a fluorescent microscope equipped with the appropriate filter combinations.

Classification—Slides were screened and classified as follows: First, slides were scanned under Bright field mode for morphology analysis (Morphology scan). During this scan, images and coordinates were captured for each and every cell on the slide. Based on the morphology information retrieved during the bright-field scan, cells of interest which are relevant for a further cytogenetical analysis (e.g., FISH) were subjected into a sub-group called “target cells”. This sub-group contains cells with abnormal morphology and/or cells which originate in the lower airways or the lungs (see examples of relevant cells in FIGS. 7A-I). It should be noted that not all cells present in the sputum sample are relevant for cytogenetical (FISH) analysis. Cells excluded from the genetic analysis are those not involved in the molecular field cancerization effect associated with lung cancer such as squamous epithelial cells (FIG. 7J) and blood cells. In the next step, the same “target cells” identified in the morphology scan were analyzed for their FISH pattern. Slides were scanned under fluorescent filters (dark field imaging) and the Duet system (Bio View, Rehovot, Israel) relocate the pre-selected “target cells” and classify them according to a seven tiered scoring system as follows: “Normal cells”—two copies of each locus-specific probe (i.e., 10q22.2-q23.1 and 3p22.1) and two copies of CEP 10 (2 green, 2 red and 2 aqua signals); “deletion 3p”—one copy of the 3p locus-specific probe (i.e., 3p22.1) is missing (1 green, 2 red and 2 aqua signals); “deletion 10q”—one copy of the 10q locus-specific probe (i.e., 10q22.2-q23.1) is missing (2 green, 1 red and 2 aqua signals); “10q-3p deletion”—one copy of the 3p locus-specific probe (i.e., 3p22.1) and one copy of the 10q locus-specific probe (i.e., 10q22.2-q23.1) is missing (1 green, 1 red and 2 aqua signals); “3p Polysomy”—more than two copies of the 3p locus-specific probe (i.e., 3p22.1) (>2 green, 2 red and 2 aqua signals); “10q Polysomy”—more than two copies of the 10q locus-specific probe (i.e., 10q22.2-q23.1) (2 green, >2 red and 2 aqua signals); and “Polysomy”—more than two copies of both 3p and 10q locus-specific probes (i.e., 10q22.2-q23.1 and 3p22.1) and CEP 10 (>2 green, >2 red and >2 aqua signals).

### Experimental and Statistics Results

Identification of genetic aberrations in relevant target cells of a sputum sample—FIGS. 8A-F provide examples of the combined staining methods (a morphological and cytogenetical staining methods) for diagnosing lung cancer. A respiratory epithelial cell stained with Papanicolaou's stain (FIG. 8A) was marked as a “target cell” being suitable for a further cytogenetic analysis. Following destaining of the morphological stain (Papanicolaou's stain in this case), the slide was subject to FISH analysis using the 3-color FISH probes (i.e., the 10q22.2-q23.1 and 3p22.1 BAC probes and CEP10) and the marked target cell was re-scanned for FISH signals. As shown in FIG. 8B, the respiratory epithelial cell exhibits two green signals corresponding to two copies of the 3p22.1 locus, two aqua signals corresponding to two copies of the chromosome 10 centromere region and one red signal corresponding to one copy of the 10q22.2-q23.1 locus, thus indicating a deletion of 10q22-23 in the analyzed cell.

An additional respiratory epithelial cell stained with Papanicolaou stain (FIG. 8C) and subsequently with the 3-color FISH probes was found to exhibit four green signals corresponding to four copies of the 3p22.1 locus, three aqua signals corresponding to three copies of the chromosome 10 centromere region and three red signals corresponding to three copy of the 10q22.2-q23.1 locus (FIG. 8D), thus indicating a polysomy in the analyzed cell.

A metaplastic cell stained with Papanicolaou stain (FIG. 8E) and subsequently with the 3-color FISH probes was found to exhibit three green signals corresponding to three copies of the 3p22.1 locus, two aqua signals corresponding to two copies of the chromosome 10 centromere region and two red signals corresponding to two copy of the 10q22.2-q23.1 locus (FIG. 8F), thus indicating 3p Polysomy in the analyzed cell.

Evaluation of sensitivity and specificity of the method of diagnosing lung cancer in sputum samples according to a scoring index—This study evaluated the sensitivity and specificity of the combined staining method according to some embodiments of the invention in diagnosing lung cancer using sputum samples (i.e., a non-invasive method). Induced sputum samples from 71 individuals were included in the study: 14 patients diagnosed with advanced stage (stage III-IV) lung cancer, 15 patients diagnosed with early stage lung cancer (stage I), 32 high-risk volunteers (heavy smokers) and 10 healthy, non-smoking, controls.

A scoring index was designed in order to represent the level of genetic abnormalities found in the sputum samples. This index reflects the percentage of FISH aberrant cells out of the “target cells” identified in the sample and the cutoff for a positive result depends on the total number of “target cells” that were scored.

Based on this index the cutoff for a positive result was as follows:

For samples which contain less than 200 target cells—the percentage of FISH aberrant cells is >10 (more than 10%).

For samples which contain between 200 and 1000 target cells—% of FISH aberrant cells >7.5.

For samples contain more than 1000 target cells—% of FISH aberrant cells >5.

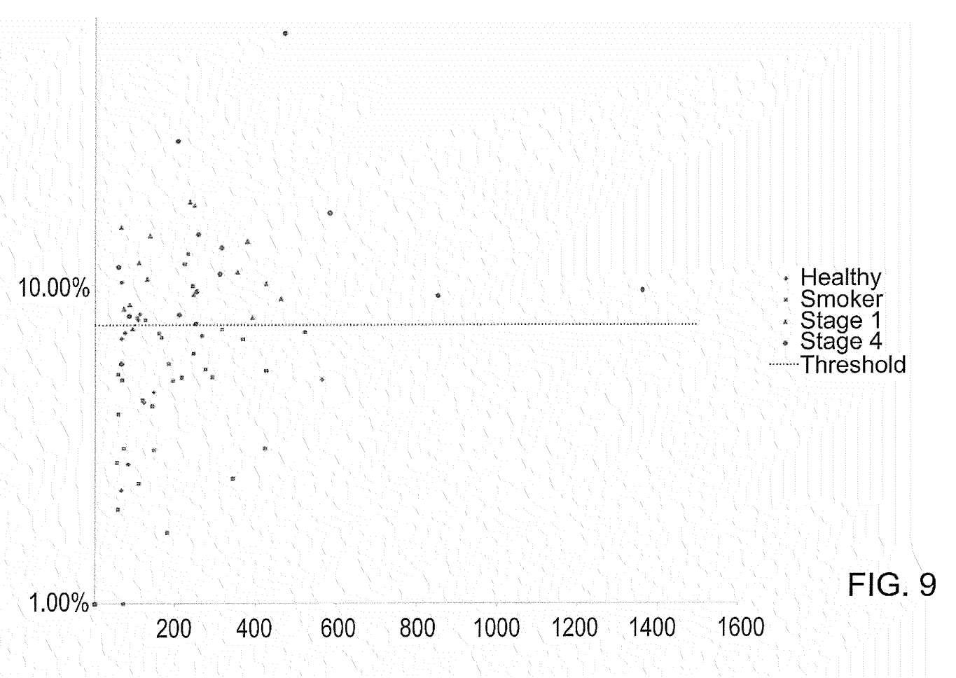

The overall results are plotted in FIG. 9. It can be seen that the majority of lung cancer patients were diagnosed with positive result while the majority of smokers and non-smoking controls exhibit negative result.

The average scores (representing the percentage of genetic abnormalities found within the target cells) for the various groups tested are described in Table 22, below.

TABLE 22
Average scores: percentages of genetic abnormalities found in target cells

| Patient Group | No. | Average score (%) |
|---|---|---|
| Advanced lung cancer | 14 | 15.29 |
| Early stage Lung cancer | 15 | 11.59 |
| Risk population (Smokers) | 32 | 5.4 |
| Healthy non-smoking controls | 10 | 5.9 |

Table 22.

The difference in the level of genetic abnormalities between lung cancer patients (including all stages) and control (smokers and non-smokers) was found to be statistically significant (p=0.008).

The sensitivity and specificity of the test is described in Table 23, below.

TABLE 23
Combined targeted analysis using FISH probes specific to 3p22.1,
10q22-23, 10 centromere

| | Lung cancer | Healthy | Sensitivity | Specificity |
|---|---|---|---|---|
| Positive result | 27 | 8 | 93.1% | 80.95% |
| Negative result | 2 | 34 | | |

Table 23.

The combined analysis method was able to detect 27/29 lung cancer patients (93.1% sensitivity) and 34/42 healthy control subjects (80.95% specificity).

Analysis and Discussion

Altogether, the present inventors provide for the first time, a non-invasive, highly accurate method of diagnosing lung cancer and/or metastatic lung cancer in sputum samples.

There are two main types of lung cancer: small cell lung cancer and non-small cell lung cancer (NSCLC). Small cell lung cancer makes up about 20% of all lung cancer cases. Non-small cell lung cancer (NSCLC) is the most common type of lung cancer. It usually grows and spreads more slowly than small cell lung cancer. There are three forms of NSCLC: (1) Adenocarcinomas, which are often found in an outer area of the lung; (2) Squamous cell carcinomas, which are usually found in the center of the lung by an air tube (bronchus); and (3) Large cell carcinomas, which may occur in any part of the lung. The large cell carcinomas tend to grow and spread faster than the other two types.

Metastatic lung cancer is the spread of cancer cells from the site of origin (e.g., a distal tissue) to the lung (e.g., via the blood stream). Common tumors that metastasize to the lungs include breast cancer, colon cancer, prostate cancer, sarcoma, bladder cancer, neuroblastoma, and Wilm's tumor. However, almost any cancer has the capacity to spread to the lungs.

Early detection of lung cancer is mandatory to reduce its extremely high mortality rate. Since sputum collection is a non-invasive test, it would be most advantageous for early cancer detection. Using sputum biomarkers could help identifying patients at a high risk for cancer-related events, such as development of pre-malignant lesions or early cancers so that these patients may be placed under intense surveillance either by fluorescent bronchoscopic examination or regular helical CT scan of lungs to detect peripheral carcinomas. Additionally this is an ideal population to benefit from chemopreventive agents and smoking cessation counseling.

### Example 5

### Fish Probes Suitable for Detection of Genetic Abnormalities in Human Chromosomes 3p22.1 and 10q22-23

TABLE 24
FISH probes on 3p22.1

| First | Last | | | |
|---|---|---|---|---|
| nucleotide | nucleotide | | Cytological | |
| coordinate | coordinate | Symbol | band/locus | Description |
| 39346219 | 39351077 | CCR8 | 3p22 | chemokine (C-C motif) receptor 8 |
| 39351380 | 39352471 | hnRNPA1p | 3p22.1 | heterogeneous nuclear |
| | | | | ribonucleoprotein A1 pseudogene |
| 39375040 | 39376736 | LOC645715 | 3p22.1 | similar to eukaryotic translation |
| | | | | elongation factor 1 alpha 2 |
| 39393013 | 39397635 | LOC100131456 | 3p22.1 | hypothetical protein LOC100131456 |
| 39399819 | 39413823 | SLC25A38 | 3p22.1 | solute carrier family 25, member 38 |
| 39422917 | 39429037 | RPSA | 3p22.2 | ribosomal protein SA |
| 39424886 | 39425034 | SNORA6 | 3p22.2 | small nucleolar RNA, H/ACA box 6 |
| 39427549 | 39427702 | SNORA62 | 3p22 | small nucleolar RNA, H/ACA box 62 |
| 39484074 | 39542863 | MOBP | 3p22.1 | myelin-associated oligodendrocyte |
| | | | | basic protein |
| 39660133 | 39661200 | LOC100132681 | 3p22.1 | hypothetical LOC100132681 |
| 39826307 | 40276816 | MYRIP | 3p22.1 | myosin VIIA and Rab interacting |
| | | | | protein |
| 40326177 | 40328919 | EIF1B | 3p22.1 | eukaryotic translation initiation factor |
| | | | | 1B |
| 40339285 | 40344043 | LOC100129750 | 3p22.1 | hypothetical LOC100129750 |
| 40403694 | 40445114 | ENTPD3 | 3p21.3 | ectonucleoside triphosphate |
| | | | | diphosphohydrolase 3 |
| 40473805 | 40478863 | RPL14 | 3p22-p21.2 | ribosomal protein L14 |
| 40493641 | 40504881 | ZNF619 | 3p22.1 | zinc finger protein 619 |
| 40522534 | 40534042 | ZNF620 | 3p22.1 | zinc finger protein 620 |
| 40541380 | 40556047 | ZNF621 | 3p22.1 | zinc finger protein 621 |
| 40612702 | 40613989 | LOC651628 | 3p22.1 | similar to Elongation factor 1-gamma |
| | | | | (EF-1-gamma) (eEF-1B gamma) |
| 40714727 | 40727054 | LOC729505 | 3p22.1 | hypothetical protein LOC729505 |
| 40777894 | 40778476 | RPS27P4 | 3p22.1 | ribosomal protein S27 pseudogene 4 |
| 41205525 | 41205737 | MRPS31P1 | 3p21.33 | mitochondrial ribosomal protein S31 |
| | | | | pseudogene 1 |
| 41215946 | 41256943 | CTNNB1 | 3p21 | catenin (cadherin-associated protein), |
| | | | | beta 1, 88 kDa |
| 41263094 | 41978664 | ULK4 | 3p22.1 | unc-51-like kinase 4 (C. elegans) |
| 41971941 | 41972294 | LOC729032 | 3p22.1 | similar to ribosomal protein L36 |
| 41990962 | 41994659 | LOC645874 | 3p22.1 | hypothetical LOC645874 |
| 42107750 | 42242272 | TRAK1 | 3p25.3-p24.1 | trafficking protein, kinesin binding 1 |
| 42162142 | 42162867 | LOC100129336 | 3p22.1 | hypothetical LOC100129336 |
| 42274322 | 42281399 | CCK | 3p22-p21.3 | cholecystokinin |
| 42284601 | 42339191 | LOC391530 | 3p22.1 | similar to sal-like 4 |
| 42357045 | 42358863 | LOC100130064 | 3p22.1 | hypothetical LOC100130064 |
| 42413579 | 42427069 | LYZL4 | 3p22.1 | lysozyme-like 4 |
| 42519121 | 42554064 | VIPR1 | 3p22 | vasoactive intestinal peptide receptor 1 |
| 42564476 | 42598432 | SEC22C | 3p22.1 | SEC22 vesicle trafficking protein |
| | | | | homolog C (S. cerevisiae) |
| 42607302 | 42611494 | SS18L2 | 3p21 | synovial sarcoma translocation gene |
| | | | | on chromosome 18-like 2 |
| 42617151 | 42665237 | NKTR | 3p23-p21 | natural killer-tumor recognition |
| | | | | sequence |
| 42675878 | 42684076 | ZBTB47 | 3p22.1 | zinc finger and BTB domain |
| | | | | containing 47 |
| 42702015 | 42708942 | KBTBD5 | 3p22.1 | kelch repeat and BTB (POZ) domain |
| | | | | containing 5 |
| 42709159 | 42718017 | HHATL | 3p22.1 | hedgehog acyltransferase-like |
| 42724878 | 42789749 | CCDC13 | 3p22.1 | coiled-coil domain containing 13 |
| 42799404 | 42821031 | HIGD1A | 3p22.1 | HIG1 domain family, member 1A |
| 42825968 | 42883779 | CCBP2 | 3p21.3 | chemokine binding protein 2 |
| 42888688 | 42892637 | CYP8B1 | 3p22-p21.3 | cytochrome P450, family 8, subfamily |
| | | | | B, polypeptide 1 |
| 42905823 | 42906802 | LOC729102 | 3p22.1 | similar to Heterogeneous nuclear |
| | | | | ribonucleoprotein A1 (Helix- |
| | | | | destabilizing protein) (Single-strand |
| | | | | RNA-binding protein) (hnRNP core |
| | | | | protein A1) (HDP) |
| 42922406 | 42934136 | ZNF662 | 3p22.1 | zinc finger protein 662 |
| 42995763 | 43074211 | LOC729085 | 3p22.1 | hypothetical protein LOC729085 |
| 43087788 | 43088201 | LOC100132850 | 3p22.1 | hypothetical protein LOC100132850 |
| 43095730 | 43122569 | C3orf39 | 3p22.1 | chromosome 3 open reading frame 39 |
| 43303008 | 43367639 | SNRK | 3p22.1 | SNF related kinase |
| 43382822 | 43638564 | TMEM16K | 3p22.1-p21.33 | transmembrane protein 16K |

Table 24: The nucleotide coordinates of polynucleotides which can be used as FISH probes capable of detecting a genetic abnormality on human chromosome 3p22.1 are provided. The first and last nucleotide coordinates (defining each polynucleotide) are given with reference to chromosome 3 of Homo sapiens Genome (Build 36.3). Chromosome: 3; Region: 39,300,001..43,600K.

TABLE 25
FISH probe on 10q22-23

| First | Last | | | |
|---|---|---|---|---|
| nucleotide | nucleotide | | Cytological | |
| coordinate | coordinate | Symbol | band/locus | Description |
| 71231650 | 71388909 | COL13A1 | 10q22 | collagen, type XIII, alpha 1 |
| 71482363 | 71542046 | H2AFY2 | 10q22 | H2A histone family, member Y2 |
| 71542036 | 71562696 | AIFM2 | 10q22.1 | apoptosis-inducing factor, mitochondrion- |
| | | | | associated, 2 |
| 71567739 | 71576502 | TYSND1 | 10q22.1 | trypsin domain containing 1 |
| 71579966 | 71600275 | SAR1A | 10q22.1 | SAR1 gene homolog A (S. cerevisiae) |
| 71593347 | 71593991 | CALM2P2 | 10q22.1 | calmodulin 2 pseudogene 2 |
| 71632592 | 71663196 | PPA1 | 10q11.1-q24 | pyrophosphatase (inorganic) 1 |
| 71684719 | 71707727 | NPFFR1 | 10q21-q22 | neuropeptide FF receptor 1 |
| 71728735 | 71812388 | LRRC20 | 10q22.1 | leucine rich repeat containing 20 |
| 71819026 | 71820495 | C10orf37 | 10q22.1 | chromosome 10 open reading frame 37 |
| 71833928 | 71853676 | EIF4EBP2 | 10q21-q22 | eukaryotic translation initiation factor 4E |
| | | | | binding protein 2 |
| 71862077 | 71871429 | NODAL | 10q22.1 | nodal homolog (mouse) |
| 71908041 | 71955804 | LOC100132225 | 10q22.1 | hypothetical protein LOC100132225 |
| 71908570 | 71998211 | KIAA1274 | 10q22.1 | KIAA1274 |
| 72027110 | 72032537 | PRF1 | 10q22 | perforin 1 (pore forming protein) |
| 72102565 | 72192203 | ADAMTS14 | 10q21 | ADAM metallopeptidase with |
| | | | | thrombospondin type 1 motif, 14 |
| 72201001 | 72215163 | C10orf27 | 10q22.1 | chromosome 10 open reading frame 27 |
| 72223929 | 72224285 | LOC338611 | 10q22.1 | similar to 40S ribosomal protein S26 |
| 72245722 | 72309873 | SGPL1 | 10q21 | sphingosine-1-phosphate lyase 1 |
| 72313273 | 72318547 | PCBD1 | 10q22 | pterin-4 alpha-carbinolamine |
| | | | | dehydratase/dimerization cofactor of |
| | | | | hepatocyte nuclear factor 1 alpha |
| 72642353 | 72729722 | UNC5B | 10q22.1 | unc-5 homolog B (C. elegans) |
| 72646987 | 72647588 | LOC728978 | 10q22.1 | hCG1818231 |
| 72749016 | 72793153 | SLC29A3 | 10q22.1 | solute carrier family 29 (nucleoside |
| | | | | transporters), member 3 |
| 72826697 | 73245710 | CDH23 | 10q21-q22 | cadherin-like 23 |
| 73141464 | 73169435 | C10orf105 | 10q22.1 | chromosome 10 open reading frame 105 |
| 73177319 | 73203343 | C10orf54 | 10q22.1 | chromosome 10 open reading frame 54 |
| 73246061 | 73281088 | PSAP | 10q21-q22 | prosaposin (variant Gaucher disease and |
| | | | | variant metachromatic leukodystrophy) |
| 73394126 | 73443318 | CHST3 | 10q22.1 | carbohydrate (chondroitin 6) |
| | | | | sulfotransferase 3 |
| 73488799 | 73518773 | SPOCK2 | 10pter-q25.3 | sparc/osteonectin, cwcv and kazal-like |
| | | | | domains proteoglycan (testican) 2 |
| 73526284 | 73645700 | ASCC1 | 10pter-q25.3 | activating signal cointegrator 1 complex |
| | | | | subunit 1 |
| 73645812 | 73665624 | C10orf104 | 10q22.1 | chromosome 10 open reading frame 104 |
| 73703683 | 73705803 | DDIT4 | 10pter-q26.12 | DNA-damage-inducible transcript 4 |
| 73762594 | 73784875 | DNAJB12 | 10q22.1 | DnaJ (Hsp40) homolog, subfamily B, |
| | | | | member 12 |
| 73797104 | 74055905 | CBARA1 | 10q22.1 | calcium binding atopy-related autoantigen 1 |
| 74066261 | 74066541 | LOC100129990 | 10q22.1 | hypothetical LOC100129990 |
| 74121895 | 74317458 | CCDC109A | 10q22.1 | coiled-coil domain containing 109A |
| 74323345 | 74362793 | OIT3 | 10q22.1 | oncoprotein induced transcript 3 |
| 74347072 | 74348298 | LOC100131044 | 10q22.1 | hypothetical LOC100131044 |
| 74364944 | 74384516 | PLA2G12B | 10q22.1 | phospholipase A2, group XIIB |
| 74435555 | 74436377 | LOC729046 | 10q22.1 | similar to 60S ribosomal protein L17 |
| | | | | (L23) |
| 74436981 | 74526630 | P4HA1 | 10q21.3-q23.1 | procollagen-proline, 2-oxoglutarate 4- |
| | | | | dioxygenase (proline 4-hydroxylase), |
| | | | | alpha polypeptide I |
| 74540216 | 74561587 | NUDT13 | 10q22.1 | nudix (nucleoside diphosphate linked |
| | | | | moiety X)-type motif 13 |
| 74564289 | 74597823 | ECD | 10q22.1 | ecdysoneless homolog (Drosophila) |
| 74597883 | 74674446 | FAM149B1 | 10q22.2 | family with sequence similarity 149, |
| | | | | member B1 |
| 74672588 | 74677031 | DNAJC9 | 10q22.2 | DnaJ (Hsp40) homolog, subfamily C, |
| | | | | member 9 |
| 74678607 | 74682457 | MRPS16 | 10q22.1 | mitochondrial ribosomal protein S16 |
| 74683526 | 74788618 | TTC18 | 10q22.2 | tetratricopeptide repeat domain 18 |
| 74805210 | 74843809 | ANXA7 | 10q21.1-q21.2 | annexin A7 |
| 74851969 | 74852465 | LOC100131526 | 10q22.2 | similar to ribosomal protein L26 |
| 74853343 | 74863325 | ZMYND17 | 10q22.2 | zinc finger, MYND-type containing 17 |
| 74866618 | 74925758 | PPP3CB | 10q21-q22 | protein phosphatase 3 (formerly 2B), |
| | | | | catalytic subunit, beta isoform |
| 74927302 | 75005439 | USP54 | 10q22.2 | ubiquitin specific peptidase 54 |
| 75061418 | 75071521 | MYOZ1 | 10q22.1 | myozenin 1 |
| 75075296 | 75080793 | SYNPO2L | 10q22.2 | synaptopodin 2-like |
| 75104037 | 75138147 | CTGLF2 | 10q22.2 | centaurin, gamma-like family, member 2 |
| 75146848 | 75160272 | LOC729096 | 10q22.2 | similar to BMS1-like, ribosome assembly |
| | | | | protein |
| 75160840 | 75167175 | GLUDP3 | 10q22.1 | glutamate dehydrogenase pseudogene 3 |
| 75174138 | 75201925 | SEC24C | 10q22.2 | SEC24 related gene family, member C (S. cerevisiae) |
| 75202055 | 75205983 | FUT11 | 10q22.2 | fucosyltransferase 11 (alpha (1,3) |
| | | | | fucosyltransferase) |
| 75211834 | 75213137 | CHCHD1 | 10q22.2 | coiled-coil-helix-coiled-coil-helix domain |
| | | | | containing 1 |
| 75215611 | 75231557 | KIAA0913 | 10q22.2 | KIAA0913 |
| 75231675 | 75241348 | NDST2 | 10q22 | N-deacetylase/N-sulfotransferase |
| | | | | (heparan glucosaminyl) 2 |
| 75242265 | 75304349 | CAMK2G | 10q22 | calcium/calmodulin-dependent protein |
| | | | | kinase (CaM kinase) II gamma |
| 75339740 | 75341994 | C10orf55 | 10q22.2 | chromosome 10 open reading frame 55 |
| 75340896 | 75347261 | PLAU | 10q24 | plasminogen activator, urokinase |
| 75427878 | 75549924 | VCL | 10q22.1-q23 | vinculin |
| 75550021 | 75580832 | AP3M1 | 10q22.2 | adaptor-related protein complex 3, mu 1 |
| | | | | subunit |
| 75580971 | 76139066 | ADK | 10q22 | adenosine kinase |
| 75852288 | 75854299 | LOC729142 | 10q22.2 | similar to Ras-related protein Rab-5C |
| 75957348 | 75957717 | MRPL35P3 | 10q22.2 | mitochondrial ribosomal protein L35 |
| | | | | pseudogene 3 |
| 76174146 | 76178502 | LOC645646 | 10q22.2 | hypothetical LOC645646 |
| 76256385 | 76462645 | MYST4 | 10q22.2 | MYST histone acetyltransferase |
| | | | | (monocytic leukemia) 4 |
| 76467600 | 76488278 | DUPD1 | 10q22.2 | dual specificity phosphatase and pro |
| | | | | isomerase domain containing 1 |
| 76518757 | 76519608 | PPIAP13 | 10q11.2-q23 | peptidylprolyl isomerase A (cyclophilin |
| | | | | A) pseudogene 13 |
| 76524196 | 76538976 | DUSP13 | 10q22.2 | dual specificity phosphatase 13 |
| 76541471 | 76606455 | SAMD8 | 10q22.2 | sterile alpha motif domain containing 8 |
| 76640569 | 76661212 | VDAC2 | 10q22 | voltage-dependent anion channel 2 |
| 76663735 | 76665776 | COMTD1 | 10q22.2 | catechol-O-methyltransferase domain |
| | | | | containing 1 |
| 76811981 | 76812669 | SPA17P1 | 10q22.2 | sperm autoantigenic protein 17 |
| | | | | pseudogene 1 |
| 76825430 | 76828717 | LOC439985 | 10q22.2 | hypothetical gene supported by |
| | | | | AK125693 |
| 76827915 | 76831431 | ZNF503 | 10q22.2 | zinc finger protein 503 |
| 76832118 | 76838072 | LOC100131213 | 10q22.2 | hypothetical protein LOC100131213 |
| 76982221 | 76982316 | MIRN606 | 10q22.2 | microRNA 606 |
| 77212525 | 77987136 | C10orf11 | 10q22.2-q22.3 | chromosome 10 open reading frame 11 |
| 78299366 | 79067583 | KCNMA1 | 10q22.3 | potassium large conductance calcium- |
| | | | | activated channel, subfamily M, alpha |
| | | | | member 1 |
| 78884873 | 78886481 | LOC729187 | 10q22.3 | hypothetical protein LOC729187 |
| 79158533 | 79164538 | LOC399783 | 10q22.3 | hypothetical gene supported by |
| | | | | NM_018181 |
| 79168007 | 79168816 | LOC100129156 | 10q22.3 | hypothetical LOC100129156 |
| 79209259 | 79211739 | LOC340780 | 10q22.3 | inosine monophosphate dehydrogenase 1 |
| | | | | pseudogene |
| 79220555 | 79356354 | DLG5 | 10q23 | discs, large homolog 5 (Drosophila) |
| 79296672 | 79298015 | LOC100131132 | 10q22.3 | hypothetical protein LOC100131132 |
| 79357445 | 79358388 | LOC100128292 | 10q22.3 | hypothetical LOC100128292 |
| 79405900 | 79459265 | POLR3A | 10q22-q23 | polymerase (RNA) III (DNA directed) |
| | | | | polypeptide A, 155 kDa |
| 79463580 | 79470480 | RPS24 | 10q22-q23 | ribosomal protein S24 |
| 79498517 | 79500788 | LOC401646 | 10q22.3 | similar to Guanine nucleotide-binding |
| | | | | protein G(i), alpha-2 subunit (Adenylate |
| | | | | cyclase-inhibiting G alpha protein) |
| 79836307 | 80014689 | LOC100132987 | 10q22.3 | hypothetical protein LOC100132987 |
| 80631347 | 80746279 | ZMIZ1 | 10q22.3 | zinc finger, MIZ-type containing 1 |
| 80777226 | 80785096 | PPIF | 10q22-q23 | peptidylprolyl isomerase F (cyclophilin F) |
| 80812087 | 80875389 | C10orf56 | 10q22.3 | chromosome 10 open reading frame 56 |
| 80928750 | 80929874 | TPRX1P1 | 10q22.3 | tetra-peptide repeat homeobox 1 |
| | | | | pseudogene 1 |
| 80935734 | 80940684 | LOC729815 | 10q22.3 | hypothetical protein LOC729815 |
| 80942363 | 80946202 | EIF5AL1 | 10q22.3 | eukaryotic translation initiation factor 5A- |
| | | | | like 1 |
| 80985614 | 80990169 | SFTPA2 | 10q22.3 | surfactant, pulmonary-associated protein |
| | | | | A2 |
| 81040722 | 81045208 | SFTPA1 | 10q22.3 | surfactant, pulmonary-associated protein |
| | | | | A1 |
| 81057013 | 81115198 | LOC650623 | 10q22.3 | hypothetical LOC650623 |
| 81131411 | 81143340 | FAM22B | 10q22.3 | family with sequence similarity 22, |
| | | | | member B |
| 81248737 | 81249661 | TPRX1P2 | 10q22.3 | tetra-peptide repeat homeobox 1 |
| | | | | pseudogene 2 |
| 81255626 | 81260577 | LOC727749 | 10q22.3 | hypothetical protein LOC727749 |
| 81262290 | 81262832 | EIF5AP1 | 10q23.3 | eukaryotic translation initiation factor 5A |
| | | | | pseudogene 1 |
| 81305573 | 81310114 | SFTPA2B | 10q22.3 | surfactant, pulmonary-associated protein |
| | | | | A2B |
| 81345039 | 81361329 | LOC100132477 | 10q22.3 | similar to surfactant, pulmonary- |
| | | | | associated protein A2 |
| 81360664 | 81363921 | SFTPA1B | 10q22.3 | surfactant, pulmonary-associated protein |
| | | | | A1B |
| 81376936 | 81435395 | LOC642300 | 10q22.3 | hypothetical LOC642300 |
| 81451610 | 81462705 | FAM22C | 10q22.3 | family with sequence similarity 22, |
| | | | | member C |
| 81575260 | 81578077 | LOC642361 | 10q22.3 | hypothetical LOC642361 |
| 81589513 | 81604115 | FAM22E | 10q22.3 | family with sequence similarity 22, |
| | | | | member E |
| 81619829 | 81624732 | LOC642413 | 10q22.3 | similar to Cathepsin L precursor (Major |
| | | | | excreted protein) (MEP) |
| 81641099 | 81642911 | LOC100132402 | 10q22.3 | similar to PGGT1B protein |
| 81669914 | 81672855 | MBL1P1 | 10q22.2-q22.3 | mannose-binding lectin (protein A) 1, |
| | | | | pseudogene 1 |
| 81687476 | 81698841 | SFTPD | 10q22.2-q23.1 | surfactant, pulmonary-associated protein D |
| 81731575 | 81733155 | LOC100130879 | 10q22.3 | hypothetical LOC100130879 |
| 81774447 | 81778155 | LOC642521 | 10q22.3 | similar to nuclear DNA-binding protein |
| 81781664 | 81782188 | LOC642538 | 10q22.3 | similar to nuclear DNA-binding protein |
| 81790318 | 81790779 | LOC727879 | 10q22.3 | similar to nuclear DNA-binding protein |
| 81828406 | 81842286 | C10orf57 | 10q22.3 | chromosome 10 open reading frame 57 |
| 81882238 | 81894766 | PLAC9 | 10q22.3 | placenta-specific 9 |
| 81904860 | 81955308 | ANXA11 | 10q23 | annexin A11 |
| 81996955 | 81997419 | EIF5AL3 | 10q22.3 | eukaryotic translation initiation factor 5A- |
| | | | | like 3 |
| 82002463 | 82003375 | LOC100130698 | 10q23.1 | hypothetical protein LOC100130698 |
| 82021556 | 82039414 | MAT1A | 10q22 | methionine adenosyltransferase I, alpha |
| 82085841 | 82106480 | DYDC1 | 10q23.1 | DPY30 domain containing 1 |
| 82106538 | 82117809 | DYDC2 | 10q23.1 | DPY30 domain containing 2 |
| 82158222 | 82182733 | C10orf58 | 10q23.1 | chromosome 10 open reading frame 58 |
| 82204047 | 82269366 | TSPAN14 | 10q23.1 | tetraspanin 14 |
| 82279333 | 82285678 | LOC727923 | 10q23.1 | hypothetical protein LOC727923 |
| 82287638 | 82396296 | SH2D4B | 10q23.1 | SH2 domain containing 4B |
| 82399492 | 82403468 | LOC642666 | 10q23.1 | hypothetical LOC642666 |
| 82466219 | 82466804 | LOC100128756 | 10q23.1 | hypothetical LOC100128756 |
| 82525688 | 82527904 | LOC647532 | 10q23.1 | phenylalanine-tRNA synthetase-like, beta |
| | | | | subunit pseudogene |
| 82886276 | 82887071 | LOC389990 | 10q23.1 | hypothetical LOC389990 |
| 83251196 | 83253047 | LOC727960 | 10q23.1 | hypothetical protein LOC727960 |
| 83625077 | 84735341 | NRG3 | 10q22-q23 | neuregulin 3 |
| 85061367 | 85064419 | LOC728027 | 10q23.1 | similar to serine/threonine kinase |
| 85422142 | 85425169 | LOC728050 | 10q23.1 | hypothetical protein LOC728050 |
| 85889165 | 85903291 | GHITM | 10q23.1 | growth hormone inducible |
| | | | | transmembrane protein |
| 85916964 | 85921704 | LOC642934 | 10q23.1 | hypothetical protein LOC642934 |
| 85923534 | 85935030 | C10orf99 | 10q23.1 | chromosome 10 open reading frame 99 |
| 85944497 | 85967102 | PCDH21 | 10q22.1-q22.3 | protocadherin 21 |
| 85970221 | 85975264 | LRIT2 | 10q23.1 | leucine-rich repeat, immunoglobulin-like |
| | | | | and transmembrane domains 2 |
| 85981256 | 85991197 | LRIT1 | 10q23 | leucine-rich repeat, immunoglobulin-like |
| | | | | and transmembrane domains 1 |
| 85994789 | 86008924 | RGR | 10q23 | retinal G protein coupled receptor |
| 86078390 | 86268256 | KIAA1128 | 10q23.1 | KIAA1128 |
| 86202717 | 86217041 | LOC100130759 | 10q23.1 | hypothetical LOC100130759 |
| 86298196 | 86311057 | LOC100131699 | 10q23.1 | hypothetical protein LOC100131699 |
| 86310158 | 86311061 | LOC439992 | 10q23.1 | similar to ribosomal protein S3a |
| 87349292 | 88116230 | GRID1 | 10q22 | glutamate receptor, ionotropic, delta 1 |
| 88014430 | 88014524 | MIRN346 | 10q23.2 | microRNA 346 |
| 88184993 | 88271521 | WAPAL | 10q23.2 | wings apart-like homolog (Drosophila) |
| 88380406 | 88381458 | RPL7AP8 | 10q23.2 | ribosomal protein L7a pseudogene 8 |
| 88404294 | 88416197 | OPN4 | 10q22 | opsin 4 (melanopsin) |
| 88418301 | 88485805 | LDB3 | 10q22.3-q23.2 | LIM domain binding 3 |
| 88505000 | 88625802 | LOC100132592 | 10q23.2 | hypothetical protein LOC100132592 |
| 88506376 | 88674925 | BMPR1A | 10q22.3 | bone morphogenetic protein receptor, |
| | | | | type IA |
| 88685576 | 88707352 | MMRN2 | 10q23.2 | multimerin 2 |
| 88708395 | 88712995 | SNCG | 10q23.2-q23.3 | synuclein, gamma (breast cancer-specific |
| | | | | protein 1) |
| 88715062 | 88719590 | LOC100128309 | 10q23.2 | hypothetical protein LOC100128309 |
| 88718168 | 88720646 | C10orf116 | 10q23.2 | chromosome 10 open reading frame 116 |
| 88718850 | 88720977 | LOC100133190 | 10q23.2 | hypothetical protein LOC100133190 |
| 88720478 | 88759940 | KIAA1975 | 10q23.2 | KIAA1975 protein similar to MRIP2 |
| 88741990 | 88750172 | BMS1P3 | 10q23.2 | BMS1 pseudogene 3 |
| 88770026 | 88774469 | FAM25A | 10q23.2 | family with sequence similarity 25, |
| | | | | member A |
| 88800223 | 88844603 | GLUD1 | 10q23.3 | glutamate dehydrogenase 1 |
| 88844933 | 88941202 | FAM35A | 10q23.2 | family with sequence similarity 35, |
| | | | | member A |
| 88975185 | 88984715 | FAM22A | 10q23.2 | family with sequence similarity 22, |
| | | | | member A |
| 88984971 | 89086722 | LOC728190 | 10q23.2 | hypothetical protein LOC728190 |
| 89091305 | 89094121 | LOC439994 | 10q23.2 | hypothetical gene supported by |
| | | | | AF064843; AK025716 |
| 89107457 | 89120432 | FAM22D | 10q23.2 | family with sequence similarity 22, |
| | | | | member D |
| 89133045 | 89141033 | LOC118945 | 10q23.2 | similar to Cathepsin L precursor (Major |
| | | | | excreted protein) (MEP) |
| 89254633 | 89303125 | MINPP1 | 10q23 | multiple inositol polyphosphate histidine |
| | | | | phosphatase, 1 |
| 89392343 | 89392763 | LOC100129552 | 10q23.2 | similar to ribosomal protein S26 |
| 89409456 | 89497442 | PAPSS2 | 10q23-q24 | 3′-phosphoadenosine 5′-phosphosulfate |
| | | | | synthase 2 |
| 89502855 | 89567897 | ATAD1 | 10q23.2 | ATPase family, AAA domain containing 1 |
| 89567950 | 89594928 | CFLP1 | 10q23.2 | cofilin pseudogene 1 |
| 89613175 | 89718512 | PTEN | 10q23.3 | phosphatase and tensin homolog (mutated |
| | | | | in multiple advanced cancers 1) |
| 89796068 | 89815704 | LOC100128990 | 10q23.31 | hypothetical LOC100128990 |
| 90023601 | 90332988 | C10orf59 | 10q23.31 | chromosome 10 open reading frame 59 |
| 90336499 | 90356712 | LIPJ | 10q23.31 | lipase, family member J |
| 90368319 | 90368672 | LOC389992 | 10q23.31 | similar to 60S ribosomal protein L7 |
| 90414074 | 90428552 | LIPF | 10q23.31 | lipase, gastric |
| 90474281 | 90502493 | LIPK | 10q23.31 | lipase, family member K |
| 90511143 | 90527979 | LIPN | 10q23.31 | lipase, family member N |
| 90535305 | 90536693 | LOC100130253 | 10q23.31 | hypothetical LOC100130253 |
| 90552467 | 90570238 | LIPM | 10q23.31 | lipase, family member M |
| 90569636 | 90601712 | ANKRD22 | 10q23.31 | ankyrin repeat domain 22 |
| 90626043 | 90627282 | LOC100132487 | 10q23.31 | hypothetical LOC100132487 |
| 90630006 | 90673224 | STAMBPL1 | 10q23.31 | STAM binding protein-like 1 |
| 90682574 | 90684664 | LOC100132116 | 10q23.31 | hypothetical protein LOC100132116 |
| 90684811 | 90702491 | ACTA2 | 10q23.3 | actin, alpha 2, smooth muscle, aorta |
| 90740268 | 90765522 | FAS | 10q24.1 | Fas (TNF receptor superfamily, member |
| | | | | 6) |
| 90955674 | 90957051 | CH25H | 10q23 | cholesterol 25-hydroxylase |
| 90963309 | 91001640 | LIPA | 10q23.2-q23.3 | lipase A, lysosomal acid, cholesterol |
| | | | | esterase (Wolman disease) |
| 91051686 | 91059013 | IFIT2 | 10q23-q25 | interferon-induced protein with |
| | | | | tetratricopeptide repeats 2 |
| 91077733 | 91090314 | IFIT3 | 10q24 | interferon-induced protein with |
| | | | | tetratricopeptide repeats 3 |
| 91110025 | 91113217 | LOC100128465 | 10q23.31 | hypothetical LOC100128465 |
| 91127793 | 91134942 | IFIT1L | 10q23.31 | interferon-induced protein with |
| | | | | tetratricopeptide repeats 1-like |
| 91142302 | 91153725 | IFIT1 | 10q25-q26 | interferon-induced protein with |
| | | | | tetratricopeptide repeats 1 |
| 91164419 | 91170733 | IFIT5 | 10q23.31 | interferon-induced protein with |
| | | | | tetratricopeptide repeats 5 |
| 91180036 | 91285293 | SLC16A12 | 10q23.31 | solute carrier family 16, member 12 |
| | | | | (monocarboxylic acid transporter 12) |
| 91332729 | 91395195 | PANK1 | 10q23.31 | pantothenate kinase 1 |
| 91342483 | 91342563 | MIRN107 | 10q23.31 | microRNA 107 |
| 91441037 | 91447665 | FLJ37201 | 10q23.31 | hypothetical protein FLJ37201 |
| 91451347 | 91524680 | MPHOSPH1 | 10q23.31 | M-phase phosphoprotein 1 |
| 91579661 | 91587441 | LOC643529 | 10q23.31 | hCG2024094 |
| 91728454 | 91728807 | LOC119358 | 10q23.31 | similar to small nuclear ribonucleoprotein |
| | | | | D2 |
| 92490555 | 92607651 | HTR7 | 10q21-q24 | 5-hydroxytryptamine (serotonin) receptor |
| | | | | 7 (adenylate cyclase-coupled) |
| 92621689 | 92658292 | RPP30 | 10q23.31 | ribonuclease P/MRP 30 kDa subunit |
| 92661837 | 92671012 | ANKRD1 | 10q23.31 | ankyrin repeat domain 1 (cardiac muscle) |
| 92710655 | 92773155 | LOC643599 | 10q23.31 | hypothetical LOC643599 |
| 92802788 | 92804816 | LOC100131370 | 10q23.31 | hypothetical LOC100131370 |
| 92901732 | 92902820 | NUDT9P1 | 10q23.32 | nudix (nucleoside diphosphate linked |
| | | | | moiety X)-type motif 9 pseudogene 1 |
| 92970349 | 93034000 | PCGF5 | 10q23.32 | polycomb group ring finger 5 |
| 93160081 | 93264500 | HECTD2 | 10q23.32 | HECT domain containing 2 |
| 93378179 | 93382838 | PPP1R3C | 10q23-q24 | protein phosphatase 1, regulatory |
| | | | | (inhibitor) subunit 3C |
| 93416517 | 93417519 | LOC441572 | 10q23.32 | similar to Glyceraldehyde-3-phosphate |
| | | | | dehydrogenase (GAPDH) |
| 93515636 | 93516942 | LOC100128043 | 10q23.32 | hypothetical LOC100128043 |
| 93548049 | 93615012 | TNKS2 | 10q23.3 | tankyrase, TRF1-interacting ankyrin- |
| | | | | related ADP-ribose polymerase 2 |
| 93555775 | 93557281 | LOC653226 | 10q23.32 | hCG1781062 |
| 93636988 | 93659106 | LOC728475 | 10q23.32 | hypothetical protein LOC728475 |
| 93656326 | 93659220 | FGFBP3 | 10q23.32 | fibroblast growth factor binding protein 3 |
| 93673716 | 93780062 | BTAF1 | 10q22-q23 | BTAF1 RNA polymerase II, B-TFIID |
| | | | | transcription factor-associated, 170 kDa |
| | | | | (Mot1 homolog, S. cerevisiae) |
| 93798379 | 94040824 | CPEB3 | 10q23.32 | cytoplasmic polyadenylation element |
| | | | | binding protein 3 |
| 93868392 | 93868977 | LOC100128302 | 10q23.32 | similar to QPS1 |
| 93965935 | 93966892 | NOLA2P1 | 10q23.32 | nucleolar protein family A, member 2 |
| | | | | pseudogene 1 |
| 94028739 | 94040955 | LOC100130772 | 10q23.32 | hypothetical protein LOC100130772 |
| 94040900 | 94103701 | MARCH5 | 10q23.32 | membrane associated ring finger |
| | | | | (C3HC4) 5 |
| 94139490 | 94170324 | LOC643768 | 10q23.32 | similar to serine/threonine kinase |
| 94203580 | 94323832 | IDE | 10q23-q25 | insulin-degrading enzyme |
| 94342971 | 94405130 | KIF11 | 10q24.1 | kinesin family member 11 |
| 94418084 | 94419685 | LOC283014 | 10q23.33 | similar to eukaryotic translation initiation |
| | | | | factor 2, subunit 2 beta, 38 kDa; |
| | | | | eukaryotic translation initiation factor 2, |
| | | | | subunit 2 (beta, 38 kD); eukaryotic |
| | | | | initiation factor 2-beta |
| 94439661 | 94445388 | HHEX | 10q23.33 | hematopoietically expressed homeobox |
| 94584450 | 94809241 | EXOC6 | 10q23.33 | exocyst complex component 6 |
| 94811011 | 94818444 | CYP26C1 | 10q23.33 | cytochrome P450, family 26, subfamily |
| | | | | C, polypeptide 1 |
| 94818482 | 94823213 | LOC100132649 | 10q23.33 | hypothetical protein LOC100132649 |
| 94823222 | 94827631 | CYP26A1 | 10q23-q24 | cytochrome P450, family 26, subfamily |
| | | | | A, polypeptide 1 |
| 94856616 | 94882150 | LOC389997 | 10q23.33 | similar to 60S ribosome subunit |
| | | | | biogenesis protein NIP7 homolog (KD93) |
| 94956401 | 94959374 | LOC387703 | 10q23.33 | similar to ATP-dependent DNA helicase |
| | | | | 2 subunit 1 (ATP-dependent DNA |
| | | | | helicase II 70 kDa subunit) (Lupus Ku |
| | | | | autoantigen protein p70) (Ku70) (70 kDa |
| | | | | subunit of Ku antigen) (Thyroid-lupus |
| | | | | autoantigen) (TLAA) (CTC box-binding |
| | | | | factor 75 kDa subunit) (CT . . . |
| 95033706 | 95034345 | LOC643863 | 10q23.33 | hypothetical LOC643863 |
| 95056176 | 95232029 | FLR1L3 | 10q24 | fer-1-like 3, myoferlin (C. elegans) |
| 95246399 | 95278839 | CEP55 | 10q23.33 | centrosomal protein 55 kDa |
| 95316412 | 95337356 | GPR120 | 10q23.33 | G protein-coupled receptor 120 |
| 95341583 | 95350983 | RBP4 | 10q23-q24 | retinol binding protein 4, plasma |
| 95362335 | 95415420 | PDE6C | 10q24 | phosphodiesterase 6C, cGMP-specific, |
| | | | | cone, alpha prime |
| 95418319 | 95452319 | C10orf4 | 10q23.33 | chromosome 10 open reading frame 4 |
| 95507668 | 95547906 | LGI1 | 10q24 | leucine-rich, glioma inactivated 1 |
| 95631033 | 95631685 | LOC100129731 | 10q23.33 | hypothetical LOC100129731 |
| 95643720 | 95652480 | TMEM20 | 10q23.33 | transmembrane protein 20 |
| 95708422 | 95709661 | PIPSL | 10q23.33 | PIP5K1A and PSMD4-like |
| 95709663 | 95711283 | LOC100101438 | 10q23.33 | phosphatidylinositol-4-phosphate 5- |
| | | | | kinase, type I, alpha pseudogene |
| 95743736 | 96078139 | PLCE1 | 10q23 | phospholipase C, epsilon 1 |
| 96029024 | 96036794 | LOC100128054 | 10q23.33 | similar to hCG2044978 |
| 96082979 | 96112673 | NOC3L | 10q23.33 | nucleolar complex associated 3 homolog |
| | | | | (S. cerevisiae) |
| 96152176 | 96286079 | TBC1D12 | 10q23.33 | TBC1 domain family, member 12 |
| 96295564 | 96351846 | HELLS | 10q24.2 | helicase, lymphoid-specific |
| 96394661 | 96397735 | LOC100130970 | 10q23.33 | hypothetical LOC100130970 |
| 96433368 | 96485514 | CYP2C18 | 10q24 | cytochrome P450, family 2, subfamily C, |
| | | | | polypeptide 18 |
| 96512453 | 96602661 | CYP2C19 | 10q24.1-q24.3 | cytochrome P450, family 2, subfamily C, |
| | | | | polypeptide 19 |
| 96688430 | 96739137 | CYP2C9 | 10q24 | cytochrome P450, family 2, subfamily C, |
| | | | | polypeptide 9 |
| 96786519 | 96819244 | CYP2C8 | 10q23.33 | cytochrome P450, family 2, subfamily C, |
| | | | | polypeptide 8 |
| 96943947 | 96976152 | C10orf129 | 10q23.33 | chromosome 10 open reading frame 129 |
| 96987319 | 97040771 | PDLIM1 | 10q22-q26.3 | PDZ and LIM domain 1 (elfin) |
| 97061520 | 97311161 | SORBS1 | 10q23.3-q24.1 | sorbin and SH3 domain containing 1 |
| 97343764 | 97345396 | LOC643981 | 10q23.33 | similar to 40S ribosomal protein S3a (V- |
| | | | | fos transformation effector protein) |
| 97355676 | 97406557 | ALDH18A1 | 10q24.3 | aldehyde dehydrogenase 18 family, |
| | | | | member A1 |
| 97413163 | 97443890 | TCTN3 | 10q23.33 | tectonic family member 3 |
| 97461526 | 97627013 | ENTPD1 | 10q24 | ectonucleoside triphosphate |
| | | | | diphosphohydrolase 1 |
| 97657712 | 97688040 | LOC100127889 | 10q23.33 | hypothetical protein LOC100127889 |
| 97711321 | 97734443 | LOC100130696 | 10q23.33 | hypothetical protein LOC100130696 |
| 97737206 | 97745041 | LOC100131720 | 10q23.33 | hypothetical protein LOC100131720 |
| 97749873 | 97782431 | CC2D2B | 10q23.33 | coiled-coil and C2 domain containing 2B |
| 97793141 | 97810612 | CCNJ | 10pter-q26.12 | cyclin J |
| 97838656 | 97839976 | LOC728558 | 10q23.33 | hypothetical protein LOC728558 |
| 97879462 | 97913507 | ZNF518A | 10q23.33 | zinc finger protein 518A |
| 97939012 | 97939990 | LOC399804 | 10q23.33 | similar to nucleophosmin 1 isoform 1 |
| 97941445 | 98021316 | BLNK | 10q23.2-q23.33 | B-cell linker |

Table 25: The nucleotide coordinates of polynucleotides which can be used as FISH probes capable of detecting a genetic abnormality on human chromosome 10q22-23 are provided. The first and last nucleotide coordinates (defining each polynucleotide) are given with reference to chromosome 10 of Homo sapiens Genome (Build 36.3). Chromosome: 10; Region: 71,300,001 . . . 98M.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. Melamed M R. Lung cancer screening results in the National Cancer Institute New York Study. Cancer 2000; 89 (11 Suppl):2356-2362.
2. Jiang F, Caraway N P, Nebiyou Bekele B, et al. Surfactant protein A gene deletion and prognostics for patients with stage I non-small lung cancer. Clin Cancer Res. 2005 Aug. 1; 11 (15):5417-24
3. Fernandez R L, Zaidi T, Caraway N, Katz R. Field cancerization I non-small cell lung cancer demonstrated by fluorescence in situ hybridization (FISH) for 3p22.1 and 10q22-23: correlation of molecular abnormalities with clinical variables. Presented at Lung SPORE Winter Meeting: Los Angeles, Calif.; Jan. 24-26, 2006.
4. Barkan G A, Caraway N P, Jiang F, et al. Comparison of molecular abnormalities in bronchial brushings and tumor touch preparations. Cancer 2005; 105 (1):35-43
5. Prindiville S A, Byers T, Hirsch F R, et al. Sputum cytological atypia as a predictor of incident lung cancer in a cohort of heavy smokers with airflow obstruction. Cancer Epidemiol Biomarkers Prey 2003; 12:987-993.
6. Shimoni, A., Nagler, A., Kaplinsky, C., Reichart, M., Avigdor, A., Hardan, I., Yeshurun, M., Daniely, M., Zilberstein, Y., Amariglio, N., Brok-Simoni, F., Rechavi, G., Trakhtenbrot, L. (2002) Chimerism testing and detection of minimal residual disease after allogeneic hematopoiatic transplantation using BioView (Duet™) combined morphological and cytological analysis. Leukemia 16, 1413-1418.
7. Hardan I., Rothman R., Gelibter A., Cohen N., Shomoni A., Sokolovsky M., Reichart M., Ishoev G., Amariglio, N. Rechavi., G. Nagler A., Trakhtenbrot, L. (2004). Determination of chromosome 13 in the bone marrow cells of patients with multiple myeloma using combined morphological and FISH analysis. Experimental Hematology, 32, 254-260.
8. Daniely M., Rona R., Kaplan T., Olsfanger S., Elboim L., Zilberstain Y., Friberger A., Kidron D., Kaplan E., Lew S., Leibovitch I. (2005). Combined analysis of morphology and Fluorescence in situ hybridization significantly increases accuracy of bladder cancer detection in voided urine samples. Urology 66, 1354-1359.
9. WO0212563A2 to KATZ, R et al.;
10. WO0626714 to KATZ, R et al.;
11. WO07087612A2 to KATZ, R et al.;
12. WO0049391A1;
13. U.S. Pat. Appl. No. 20060078885 to Katz R., et al.;
14. U.S. 2004-0197839 to Daniely M. et al.;
15. Nymark P., et al., Cancer Research 66, 5737-5743, 2006;
16. Girard L., et al., Cancer Research 60, 4894-4906, 2000.

1. A method of identifying a genetically abnormal cell in a sputum sample, the method comprising:
(a) staining a sputum sample using a morphological stain so as to identify a lower airway tract cell or lung cell in said sputum sample; and
(b) staining said sputum sample using fluorescent in situ hybridization (FISH) so as to identify in said lower airway tract cell or lung cell a genetic abnormality in at least one of human chromosome 3p22.1 and 10q22-23,
thereby identifying the genetically abnormal cell in the sputum sample.

2. A method of diagnosing lung cancer in a subject comprising:
(a) staining a sputum sample of the subject with a morphological stain so as to identify lower airway tract cells or lung cells in said sputum sample;
(b) staining said sputum sample with FISH so as to identify a genetic abnormality in at least one of human chromosome 3p22.1 and 10q22-23 in said lower airway tract cells or lung cells identified in step (a),
wherein a percentage or number above a predetermined threshold of said lower airway tract cells or lung cells having said genetic abnormality is indicative of the lung cancer,
thereby diagnosing the lung cancer in the subject.

3. A method of diagnosing lung cancer in a subject, comprising:
(a) staining a sputum sample with a morphological stain so as to identify lower airway tract cells or lung cells in said sputum sample;
(b) staining said sputum sample with FISH so as to identify a genetic abnormality in at least one of human chromosome 3p22.1 and 10q22-23 in cells of said sputum sample,
wherein a percentage or number above a predetermined threshold of:
(i) said lower airway tract cells or lung cells of said sputum sample identified in step (a) having said genetic abnormality; or
(ii) said cells of said sputum sample having said genetic abnormality
is indicative of the lung cancer,
thereby diagnosing the lung cancer in the subject.

4. A kit for diagnosing lung cancer, the kit comprising a morphological stain and a FISH probe specific for human chromosome 3p22.1 and/or 10q22-23.

5. The method of claim 3, wherein said cells of said sputum sample comprise lower airway tract cells, lung cells, squamous epithelial cells and/or blood cells.

6. The method of claim 2, further comprising:
(c) imaging said lower airway tract cell or lung cell with at least two imaging modalities, thereby identifying said genetic abnormality in said cell.

7. The method of claim 3, further comprising:
(c) imaging said lower airway tract cells or lung cells with at least two imaging modalities, thereby identifying genetic abnormalities in said lower airway tract cells or lung cells.

8. The method of claim 6, wherein said imaging is effected simultaneously.

9. The method of claim 6, wherein said imaging is effected using an automated image analysis device capable of at least dual imaging.

10. The kit of claim 4, further comprises instructions for use in diagnosing lung cancer.

11. The kit of claim 4, for diagnosing lung cancer in a sputum sample.

12. The method of claim 2, wherein said sputum sample is induced by saline inhalation.

13. The kit of claim 10, wherein said instructions comprise a predetermined threshold of a percentage or number of lower airway tract cell or lung cell having a genetically abnormality in said human chromosome 3p22.1 and/or 10q22-23 which is indicative of positive diagnosis of lung cancer.

14. The method of claim 2, wherein said sputum sample is obtained from a subject at risk of developing lung cancer.

15. The method of claim 2, wherein the subject is at risk of developing lung cancer.

16. The method of claim 2, wherein said lung cancer comprises non-small cell lung cancer.

17. The method of claim 2, wherein said lung cancer comprises metastatic lung cancer.

18. The method of claim 2, wherein said morphological stain is selected from the group consisting of May-Grünwald-Giemsa, Giemsa, Papanicolaou, Diff-Quick, and Hematoxylin-Eosin.

19. The method of claim 1, wherein said FISH is effected using a FISH probe specific to human chromosome 3p22.1 and a FISH probe specific to human chromosome 10q22-23.

20. The method of claim 2, wherein said FISH is effected using at least three FISH probes.

21. The method of claim 2, wherein said FISH is effected using at least four FISH probes.

22. The method of claim 2, wherein said FISH is effected using a FISH probe specific to human chromosome 3p22.1, a FISH probe specific to human chromosome 10q22-23 and a FISH probe specific to human chromosome 10.

23. The method of claim 2, wherein said FISH is effected using a FISH probe specific to human chromosome 3p22.1, a FISH probe specific to human chromosome 10q22-23 and a FISH probe specific to human chromosome 3.

24. The method of claim 2, wherein said FISH is effected using a FISH probe selected from a group of probes specific to human chromosome 3p22.1, human chromosome 10q22-23, human chromosome 3 and human chromosome 10.
